(19) United States
(12) Patent Application Publication
(10) Pub. No.: US 2006/0234997 A1
(43) Pub. Date: Oct. 19, 2006

(54) Tetrahydro-pyridoazepin-8-ones and related compounds for the treatment of schizophrenia

(75) Inventors: David Favor, Ypsilanti, MI (US); Douglas S. Johnson, Dexter, MI (US); Joseph Thomas Repine, Ann Arbor, MI (US); Andrew David White, Pinckney, MI (US)

Correspondence Address: WARNER-LAMBERT COMPANY, 2800 PLYMOUTH RD, ANN ARBOR, MI, 48105, US

(73) Assignee: WARNER-LAMBERT COMPANY LLC

(21) Appl. No.: 11/395,931

(22) Filed: Mar. 31, 2006

Related U.S. Application Data
(60) Provisional application No. 60/667,447, filed on Apr. 1, 2005.

(51) Int. Cl. A61K 31/5513; A61K 31/55

(52) U.S. Cl. 514/212.06; 514/212.07; 514/221

(57) ABSTRACT

Compounds of formula 1 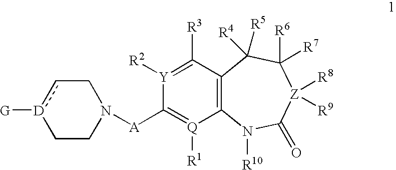
are disclosed, wherein G, D, A, Q, Y, Z, and R¹ through R¹⁰ are defined in the specification. Also provided are descriptions of processes for preparing compounds of formula 1, intermediates used in making the same, and pharmaceutical compositions containing such compounds and their use in the treatment of central nervous system disorders and other disorders.

### CROSS-REFERENCE TO RELATED APPLICATIONS

This application claims the benefit of U.S. Provisional Application No. 60/667,447 filed on Apr. 1, 2005.

### BACKGROUND OF THE INVENTION

This invention relates to tetrahydro-pyridoazepin-8-ones and related compounds, methods of making such compounds, pharmaceutical compositions containing them, and their use for the treatment of schizophrenia and other central nervous system (CNS) disorders or conditions.

Dopamine D2 receptor antaonists and particial agonists have been used clinically for treating central nervous system (CNS) disorders, such as Schizophrenia. For example, aripiprazole, which is a dopamine D2 receptor particial agonist, has been approved by the United States Food and Drug Administration for the indication of schizophrenia and bipolar disorder. (See, Tsuyoshi Hirose and Tetsuro Kikuchi, Aripiprazole, a novel antipsychotic agent: Dopamine D2 receptor particial agonist. J. Med. Invest. Vol. 52, Suppl.: 284-290 (2005) Aripiprazole was disclosed in U.S. Pat. No. 5,006,528. The tetrahydro-pyridoazepin-8-ones and related compounds of this invention bind to dopamine D2 receptors. Some exhibit activity as partial agonists of dopamine D2 receptors, while others exhibit activity as antagonists of such receptors. Therefore, the compounds of this invention are useful for treating CNS disorders, particularly schizophrenia and bipolar disorder. Other heterocyclic derivatives that are useful for the treatment of schizophrenia are referred to in U.S. Pat. No. 5,350,747, which issued on Sep. 27, 1994; in U.S. Pat. No. 6,127,357, which issued on Oct. 3, 2000; in WO 93/04684, which published on Mar. 18, 1993; and European patent application EP 402644A, which was published on Dec. 19, 1990. The foregoing patents, patent applications, and publications are incorporated herein by reference in their entireties.

### SUMMARY OF THE INVENTION

The present invention relates to compounds of formula 1 as shown below:

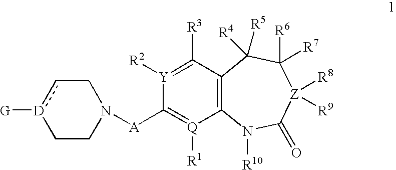

wherein G is a group selected from formula (i) or formula (ii), below:

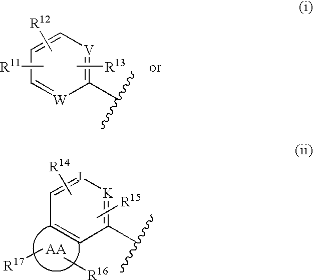

and wherein:

A is —(CH₂)ₘCH₂—, —(CH₂)ₘO—, or —(CH₂)ₘNH—, wherein m is an integer from 3 to 5, wherein two of the carbon atoms of —(CH₂)ₘCH₂— are optionally linked by a double bond, and wherein one or two of the carbon or nitrogen atoms of —(CH₂)ₘCH₂—, —(CH₂)ₘO—, and —(CH₂)ₘNH— can be substituted, optionally and independently, with a methyl or ethyl;

D is N, C, or CH, provided that when D is N, each carbon atom attached to D is attached through a single bond;

J and K are independently selected from N, CH, and C;

Q, Y, and Z are independently selected from N or C;

V and W are independently N, C, or CH;

ring AA is a saturated or unsaturated 5- 6- or 7-membered carbocyclic ring wherein one, two or three of the carbon atoms of ring AA that are not shared with the 6-membered aryl ring of group (ii) can be replaced, optionally and independently, by a nitrogen, oxygen or sulfur atom;

R¹, R², and R³ are independently selected from hydrogen, halo, cyano, hydroxy, (C₁-C₄) alkyl, and (C₁-C₄) alkoxy, wherein the alkyl moieties of the (C₁-C₄) alkyl or (C₁-C₄) alkoxy are straight or branched and can be optionally substituted with from one to three fluoro atoms and can also be optionally substituted with an amino or hydroxy substituent, provided that when Q is N, R¹ is absent and when Y is N, R² is absent;

R⁴, R⁵, R⁶, R⁷, R⁸, and R⁹ are independently selected from hydrogen, fluoro, hydroxy, (C₁-C₄) alkyl, and (C₁-C₄) alkoxy, wherein the alkyl moieties of the (C₁-C₄) alkyl or (C₁-C₄) alkoxy are straight or branched; provided that when Z is N, R⁸ cannot be fluoro or hydroxyl, and when Z is N, R⁹ is absent;

R¹⁰ is independently selected from hydrogen, (C₁-C₄) alkyl, and (C₁-C₄) acetyl, wherein the alkyl moieties of the (C₁-C₄) alkyl or (C₁-C₄) acetyl are straight or branched;

R¹¹, R¹², R¹³, R¹⁴, and R¹⁵ are independently selected from hydrogen, halo, —C(═O)CH₃, (C₁-C₄) alkyl, and (C₁-C₄) alkoxy, aryl, and aryloxy, wherein the alkyl moieties of the (C₁-C₄) alkyl, (C₁-C₄) alkoxy, and —C(═O)CH₃ groups and the aryl and aryloxy moieties can be optionally substituted with from one to three fluoro atoms and can also be optionally substituted with an amino or hydroxy substituent;

R¹⁶ and R¹⁷ are independently selected from hydrogen, halo, cyano, oxo, hydroxy, —C(═O)CH₃, (C₁-C₄) alkyl, and (C₁-C₄) alkoxy, wherein the alkyl moieties of the (C₁-C₄) alkyl, (C₁-C₄) alkoxy, and —C(═O)CH₃ groups can be optionally substituted with from one to three fluoro atoms and can also be optionally substituted with an amino or hydroxy substituent;

and the pharmaceutically acceptable salts of such compounds.

This invention also relates to a pharmaceutical composition comprising a therapeutically effective amount of a compound of the formula 1, or a pharmaceutically acceptable salt thereof, and a pharmaceutically acceptable carrier.

The compounds of formula 1 have useful pharmaceutical and medicinal properties.

The invention also relates to a pharmaceutical composition comprising a compound of formula 1, or a pharmaceutically acceptable salt, and a pharmaceutically acceptable carrier. The pharmaceutical composition is useful for treating a mammal, particularly a human, for a disorder or condition selected from single episodic or recurrent major depressive disorders, dysthymic disorders, depressive neurosis and neurotic depression, melancholic depression; atypical depression; bipolar disorder; cyclothymic disorder; conduct disorder; disruptive behavior disorder; attention deficit hyperactivity disorder; behavioral disturbances associated with mental retardation, autistic disorder, and conduct disorder; anxiety disorders; borderline personality disorder; schizophrenia and other psychotic disorders; delirium, dementia, and amnestic and other cognitive or neurodegenerative disorders; movement disorders, dyskinesias; extra-pyramidal movement disorders; chemical dependencies and additions; behavioral addictions; and ocular disorders.

The invention further relates to a pharmaceutical composition for treating a disorder or condition selected from those listed above, comprising: (a) a compound of formula 1, or a pharmaceutically acceptable salt thereof; and (b) an antidepressant or an anti-anxiety agent; and (c) a pharmaceutically acceptable carrier; wherein active agents (a) and (b) are not the same and are present in amounts that render the combination of them effective in treating said disorder or condition.

This invention also relates to a method of treating a disorder or condition in a mammal, particularly a human, the method comprising administering to the mammal in need of such treatment an effective amount of a compound according to formula 1, or a pharmaceutically acceptable salt thereof, wherein the disorder or condition is selected from those listed above.

The invention also relates to a method of treating a disorder or condition selected from those listed above, comprising administering to a mammal, particularly a human, in need of such treatment (a) a compound of formula 1, or a pharmaceutically acceptable salt thereof; and (b) an antidepressant or an anti-anxiety agent; wherein the active agents (a) and (b) are not the same and are present in amounts that render the combination of them effective in treating the disorder or condition.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “alkoxy”, as used herein, unless otherwise indicated, means “alkyl-O—,” wherein “alkyl” is as defined above. Examples of “alkoxy” groups include, but are not limited to, methoxy, ethoxy, propoxy, butoxy and pentoxy.

The term “aryl”, as used herein, unless otherwise indicated, includes an aromatic ring system with no heteroatoms as ring members, which can be either unsubstituted or substituted with one, two or three substituents selected from the group consisting of halo, (C₁-C₄)alkyl optionally substituted with from one to three fluorine atoms and (C₁-C₄)alkoxy optionally substituted with from one to three fluorine atoms.

The term “aryloxy”, as used herein, unless otherwise indicated, means “aryl-O—”, wherein “aryl” is as defined above.

The term “one or more substituents”, as used herein, refers to a number of substituents that equals from one to the maximum number of substituents possible based on the number of available bonding sites.

The terms “halo” and “halogen”, as used herein, unless otherwise indicated, include, fluoro, chloro, bromo and iodo.

The term “therapeutically effective amount,” as used herein, refers to a quantity of active agent sufficient to treat one or more of the disorders or conditions referred to above, when one or more doses of a pharmaceutical composition of the invention are administered to a subject with one or more of the disorders or conditions. In determining what constitutes a therapeutically effective amount of an active agent in a composition or delivered in a method of the present invention, a number of factors will generally be considered, including the experience of the medical practitioner or veterinarian administering the composition, published clinical studies, the subject's age, sex, weight and general condition, as well as the type and extent of the disorder or condition being treated, and the use of other medications, if any, by the subject. Determination of a proper dose for a particular situation, and preparation of a pharmaceutical composition containing a suitable dose of active agent for that situation, is within the skill of the medical or veterinary arts.

The term “treating”, as used herein, refers to reversing, alleviating, inhibiting the progress of, or preventing the disorder or condition to which such term applies, or preventing one or more symptoms of such condition or disorder.

The term “treatment”, as used herein, refers to the act of treating, as “treating” is defined immediately above.

The compounds of formula 1, and the pharmaceutically acceptable salts of these compounds are referred to herein, collectively, as the “novel compounds of this invention” and the “active compounds of this invention”.

### DETAILED DESCRIPTION OF THE INVENTION

Examples of preferred embodiments of this invention are compounds of the formula 1, and their pharmaceutically acceptable salts, wherein D is N.

Other preferred embodiments of this invention are compounds of the formula 1, and their pharmaceutically acceptable salts, wherein at least one of Q and Z is N. Both Q and Z are preferably N.

Other preferred embodiments of this invention are compounds of the formula 1, and their pharmaceutically acceptable salts, wherein R¹, R², R³, R⁶, R⁷, and R¹⁰ are each H. In this embodiment, R⁴, R⁵, R⁸, and R⁹ are preferably each independently H or methyl.

Other preferred embodiments of this invention are compounds of the formula 1, and their pharmaceutically acceptable salts, wherein A is —(CH₂)ₘCH₂— or —(CH₂)ₘO— and m is an integer from 3 to 5. m is preferably 3 or 4, more preferably 4.

Other preferred embodiments of this invention are compounds of the formula 1, and their pharmaceutically acceptable salts, wherein G is a group of formula (i), and V is C or CH.

Other preferred embodiments of this invention are compounds of the formula 1, and their pharmaceutically acceptable salts, wherein G is a group of formula (i), and R¹¹, R¹², and R¹³ are independently selected from the group consisting of halo, methyl, ethyl, isopropyl, and cyclopropyl. When any one of R¹¹, R¹², and R¹³ is halo, it is preferably Cl or F.

Other preferred embodiments of this invention are compounds of the formula 1, and their pharmaceutically acceptable salts, wherein G is a group of formula (ii), and J and K are each C or CH.

Other preferred embodiments of this invention are compounds of the formula 1, and their pharmaceutically acceptable salts, wherein G is a group of formula (ii), and ring AA is an unsaturated 6-membered carbocyclic ring.

Other preferred embodiments of this invention are compounds of the formula 1, and their pharmaceutically acceptable salts, wherein G is a group of formula (ii), wherein R¹⁴ and R¹⁵ are both H.

Other preferred embodiments of this invention are compounds of the formula 1, and their pharmaceutically acceptable salts, wherein G is a group of formula (ii), wherein R¹⁶ and R¹⁷ are independently selected from the group consisting of H, F, ═O, methyl, CN, and methoxy.

Other preferred embodiments of this invention are compounds of the formula 1, and their pharmaceutically acceptable salts, wherein G is a flouro-naphthalenyl group, preferably a 7-fluoro-naphthalen-1-yl group.

Specific embodiments of this invention include the following compounds and their pharmaceutically acceptable salts:
2-[4-(4-Naphthalen-1-yl-piperazin-1-yl)-butoxy]-5,6,7,9-tetrahydro-pyrido[2,3-b]azepin-8-one;
2-{4-[4-(2,3-dichloro-phenyl)-piperazin-1-yl]-butoxy}-5,6,7,9-tetrahydro-pyrido[2,3-b]azepin-8-one;
2-[4-(4-Chroman-8-yl-piperazin-1-yl)-butoxy]-5,6,7,9-tetrahydro-pyrido[2,3-b]azepin-8-one;
2-{4-[4-(5,6,7,8-Tetrahydro-naphthalen-1-yl)-piperazin-1-yl]-butoxy}-5,6,7,9-tetrahydro-pyrido[2,3-b]azepin-8-one;
2-[4-(4-Indan-4-yl-piperazin-1-yl)-butoxy]-5,6,7,9-tetrahydro-pyrido[2,3-b]azepin-8-one;
2-{4-[4-(2,3-Dihydro-benzofuran-7-yl)-piperazin-1-yl]-butoxy}-5,6,7,9-tetrahydro-pyrido[2,3-b]azepin-8-one;
2-{4-[4-(7-Fluoro-naphthalen-1-yl)-piperazin-1-yl]-butoxy}-5,6,7,9-tetrahydro-pyrido[2,3-b]azepin-8-one;
2-{4-[4-(3,4-Dihydro-2H-benzo[b][1,4]dioxepin-6-yl)-piperazin-1-yl]-butoxy}-5,6,7,9-tetrahydro-pyrido[2,3-b]azepin-8-one;
8-{4-[4-(8-Oxo-6,7,8,9-tetrahydro-5H-pyrido[2,3-b]azepin-2-yloxy)-butyl]-piperazin-1-yl}-naphthalene-2-carbonitrile;
2-{4-[4-(1-Methyl-2-oxo-1,2,3,4-tetrahydro-quinolin-8-yl)-piperazin-1-yl]-butoxy}-5,6,7,9-tetrahydro-pyrido[2,3-b]azepin-8-one;
2-[4-(4-Indan-4-yl-piperazin-1-yl)-butoxy]-5,6,7,9-tetrahydro-1,7,9-triaza-benzocyclohepten-8-one;
2-{4-[4-(2,3-Dichloro-phenyl)-piperazin-1-yl]-butoxy}-5,6,7,9-tetrahydro-1,7,9-triaza-benzocyclohepten-8-one;
2-[4-(4-Naphthalen-1-yl-piperazin-1-yl)-butoxy]-5,6,7,9-tetrahydro-1,7,9-triaza-benzocyclohepten-8-one;
2-{4-[4-(5,6,7,8-Tetrahydro-naphthalen-1-yl)-piperazin-1-yl]-butoxy}-5,6,7,9-tetrahydro-1,7,9-triaza-benzocyclohepten-8-one;
2-[4-(4-Chroman-8-yl-piperazin-1-yl)-butoxy]-5,6,7,9-tetrahydro-1,7,9-triaza-benzocyclohepten-8-one;
2-{4-[4-(7-Fluoro-naphthalen-1-yl)-piperazin-1-yl]-butoxy}-5,6,7,9-tetrahydro-1,7,9-triaza-benzocyclohepten-8-one;
2-{4-[4-(2,3-Dihydro-benzofuran-7-yl)-piperazin-1-yl]-butoxy}-5,6,7,9-tetrahydro-1,7,9-triaza-benzocyclohepten-8-one;
2-{4-[4-(3,4-Dihydro-2H-benzo[b][1,4]dioxepin-6-yl)-piperazin-1-yl]-butoxy}-5,6,7,9-tetrahydro-1,7,9-triaza-benzocyclohepten-8-one;
2-{4-[4-(7-Methoxy-naphthalen-1-yl)-piperazin-1-yl]-butoxy}-5,6,7,9-tetrahydro-1,7,9-triaza-benzocyclohepten-8-one;
8-{4-[4-(8-Oxo-6,7,8,9-tetrahydro-5H-1,7,9-triaza-benzocyclohepten-2-yloxy)-butyl]-piperazin-1-yl}-naphthalene-2-carbonitrile;
2-{4-[4-(1-Methyl-2-oxo-1,2,3,4-tetrahydro-quinolin-8-yl)-piperazin-1-yl]-butoxy}-5,6,7,9-tetrahydro-1,7,9-triaza-benzocyclohepten-8-one;
8-{4-[4-(2,3-Dichlorophenyl)piperazin-1-yl]butoxy}-3-methyl-1,3,4,5-tetrahydro-benzo[d][1,3]diazepin-2-one;
8-{3-[4-(2,3-Dichlorophenyl)piperazin-1-yl]propoxy}-1,3,4,5-tetrahydrobenzo[d][1,3]diazepin-2-one;
8-{4-[4-(2,3-Dichlorophenyl)piperazin-1-yl]butoxy}-1,3,4,5-tetrahydrobenzo[d][1,3]diazepin-2-one;
8-{4-[4-(2-Chloro-4-fluoro-3-methyl-phenyl)-piperazin-1-yl]-butoxy}-1,3,4,5-tetrahydro-benzo[d][1,3]diazepin-2-one;
8-{4-[4-(2-Chloro-4-fluoro-5-methyl-phenyl)-piperazin-1-yl]-butoxy}-1,3,4,5-tetrahydro-benzo[d][1,3]diazepin-2-one;
8-[4-(4-Naphthalen-1-yl-piperazin-1-yl)-butoxy]-1,3,4,5-tetrahydro-benzo[d][1,3]diazepin-2-one;
8-{4-[4-(6-Ethyl-pyridin-2-yl)-piperazin-1-yl]-butoxy}-1,3,4,5-tetrahydro-benzo[d][1,3]diazepin-2-one;
8-{4-[4-(6-Isopropyl-pyridin-2-yl)-piperazin-1-yl]-butoxy}-1,3,4,5-tetrahydro-benzo[d][1,3]diazepin-2-one;
8-{4-[4-(2-Chloro-4-fluoro-phenyl)-piperazin-1-yl]-butoxy}-1,3,4,5-tetrahydro-benzo[d][1,3]diazepin-2-one;
8-{4-[4-(2,3-Dichloro-4-fluoro-phenyl)-piperazin-1-yl]-butoxy}-1,3,4,5-tetrahydro-benzo[d][1,3]diazepin-2-one;
8-{4-[4-(6-Cyclopropyl-pyridin-2-yl)-piperazin-1-yl]-butoxy}-1,3,4,5-tetrahydro-benzo[d][1,3]diazepin-2-one;
8-{4-[4-(7-Fluoro-naphthalen-1-yl)-piperazin-1-yl]-butoxy}-1,3,4,5-tetrahydro-benzo[d][1,3]diazepin-2-one;
8-{4-[4-(2,1,3-benzothiadiazol-4-yl)piperazin-1-yl]butoxy}-1,3,4,5-tetrahydro-2H-1,3-benzodiazepin-2-one;
8-{4-[4-(5-Fluoro-naphthalen-1-yl)-piperazin-1-yl]-butoxy}-1,3,4,5-tetrahydro-benzo[d][1,3]diazepin-2-one;
8-{3-[4-(2-Methoxy-quinolin-8-yl)-piperazin-1-yl]-propoxy}-1,3,4,5-tetrahydro-benzo[d][1,3]diazepin-2-one;
8-{4-[4-(8-Fluoro-naphthalen-1-yl)-piperazin-1-yl]-butoxy}-1,3,4,5-tetrahydro-benzo[d][1,3]diazepin-2-one;
8-[3-(4-Naphthalen-1-yl-piperazin-1-yl)-propoxy]-1,3,4,5-tetrahydro-benzo[d][1,3]diazepin-2-one;
8-{3-[4-(7-Fluoro-naphthalen-1-yl)-piperazin-1-yl]-propoxy}-1,3,4,5-tetrahydro-benzo[d][1,3]diazepin-2-one;
8-[4-(4-Isochroman-8-yl-piperazin-1-yl)-butoxy]-1,3,4,5-tetrahydro-benzo[d][1,3]diazepin-2-one;
8-{3-[4-(2,3-Dichlorophenyl)piperazin-1-yl]-propoxy}-5,5-dimethyl-1,3,4,5-tetrahydrobenzo[b]azepin-2-one;
8-{4-[4-(2,3-Dichlorophenyl)piperazin-1-yl]-butoxy}-5,5-dimethyl-1,3,4,5-tetrahydrobenzo[b]azepin-2-one;
8-{5-[4-(2,3-Dichlorophenyl)piperazin-1-yl]-pentyloxy}-5,5-dimethyl-1,3,4,5-tetrahydrobenzo[b]azepin-2-one;
8-{4-[4-(2,3-Dichlorophenyl)piperazin-1-yl]butoxy}-3,3-dimethyl-1,3,4,5-tetrahydrobenzo[b]azepin-2-one;
4,4-Dimethyl-8-[4-(4-naphthalen-1-yl-piperazin-1-yl)-butoxy]-1,3,4,5-tetrahydro-benzo[d][1,3]diazepin-2-one;
4,4-Dimethyl-8-[3-(4-naphthalen-1-yl-piperazin-1-yl)-propoxy]-1,3,4,5-tetrahydro-benzo[d][1,3]diazepin-2-one;
8-{4-[4-(2,3-Dichloro-phenyl)-piperazin-1-yl]-butoxy}-1,3,4,5-tetrahydro-benzo[b]azepin-2-one;
8-{5-[4-(2,3-Dichloro-phenyl)-piperazin-1-yl]-pent-1-enyl}-1,3,4,5-tetrahydro-benzo[d][1,3]diazepin-2-one;
8-{5-[4-(2,3-Dichloro-phenyl)-piperazin-1-yl]-pentyl}-1,3,4,5-tetrahydro-benzo[d][1,3]diazepin-2-one;
8-{5-[4-(2,3-Dichlorophenyl)piperazin-1-yl]pentyl}-5,5-dimethyl-1,3,4,5-tetrahydro-benzo[d][1,3]diazepin-2-one;
8-{5-[4-(2,3-Dichlorophenyl)piperazin-1-yl]pent-1-enyl}-5,5-dimethyl-1,3,4,5-tetrahydrobenzo[b]azepin-2-one;
8-{5-[4-(2,3-Dichlorophenyl)piperazin-1-yl]pentyl}-5,5-dimethyl-1,3,4,5-tetrahydrobenzo[b]azepin-2-one.

Compounds of formula 1 may contain chiral centers and therefore may exist in different enantiomeric and diastereomeric forms. This invention relates to all optical isomers and all stereoisomers of compounds of formula 1, both as racemic mixtures and as individual enantiomers and diastereoisomers of such compounds, and mixtures thereof, and to all pharmaceutical compositions and methods of treatment defined above that contain or employ them, respectively. Individual isomers can be obtained by known methods, such as optical resolution, fractional crystallization, optically selective reaction, or chromatographic separation in the preparation of the final product or its intermediate. Individual enantiomers of the compounds of formula 1 may have advantages, as compared with the racemic mixtures of these compounds, in the treatment of various disorders or conditions.

In so far as the compounds of formula 1 are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the base compounds of this invention are readily prepared by treating the base compound with a substantially equivalent amount of the chosen mineral or organic acid in an aqueous solvent or in a suitable organic solvent, such as methanol or ethanol. Upon careful evaporation of the solvent, the desired solid salt is readily obtained. The acids which are used to prepare the pharmaceutically acceptable acid addition salts of the aforementioned base compounds of this invention are those which form non-toxic acid addition salts, i.e., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The present invention also includes isotopically labeled compounds, which are identical to those of formula 1, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula 1 and prodrugs thereof can generally be prepared by carrying out the procedures disclosed in the Schemes and/or in the Examples below, by substituting a readily available isotopically labeled reagent for a non-isotopically labeled reagent.

Compounds of this invention may be prepared as described below. Unless otherwise indicated, in the reaction schemes and discussion that follow, A, D, Y, Q, Z, V, W, J, K, ring AA, and R¹ through R¹⁷ of the formulas below are defined as above. Except where otherwise indicated, n is 3, 4, or 5 in the formulas below.

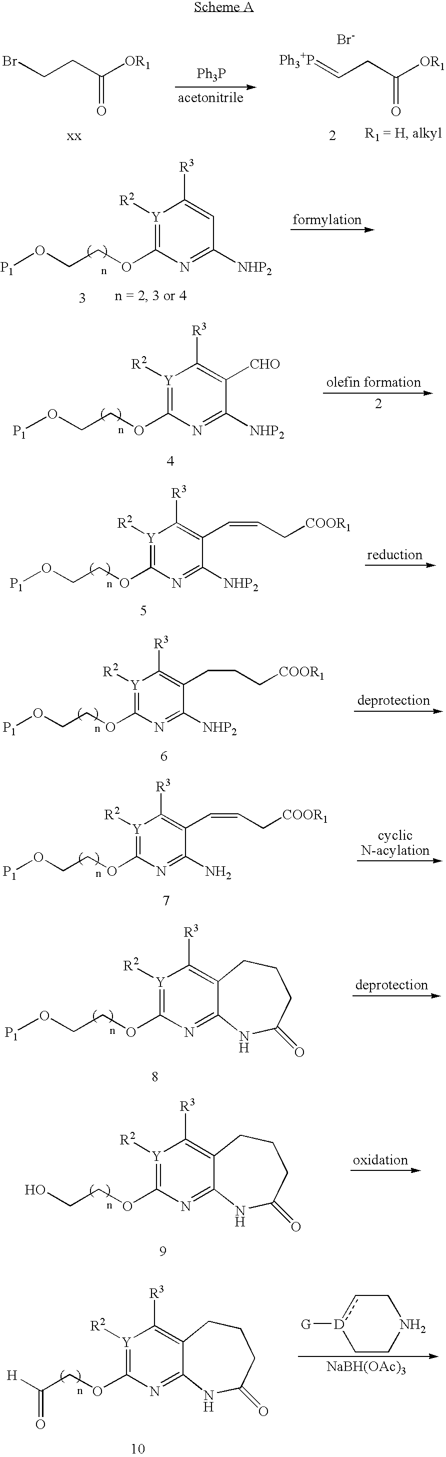

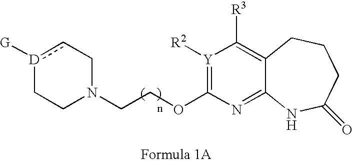

Scheme A illustrates a method for preparing compounds of formula 1A, i.e., compounds of formula 1 wherein A is —(CH₂)ₙO—, Q is N, and Z is C. This method involves preparation of a phosphonium ylide 2 for the formation of a C═C bond from an aromatic aldehyde 4 by reaction of triphenylphosphine with a suitable haloalkyl ester or acid such as a compound of formula xx. P₁ is a hydroxy-protecting group such as tetrahydropyranyl which is removable under acidic conditions. P₂ is an acyl protecting group such as 2,2-dimethyl-propionyl(pivaloyl) which is removable under protic acidic or basic conditions. Compounds of the formula 3 can be formulated by deprotonation with a strong base such as butyllithium followed by addition of dimethylformamide (DMF) to give compounds such as 4. Reaction of compounds of the formula 2 and 4 under Wittig conditions give chain extended acid or ester olefins of the formula 5. Reduction of the C═C bond is accomplished preferably by catalytic hydrogenation to give the saturated alkyl chain extended acid or ester 6. Use of intermediates where R₁ is alkyl can simultaneously be hydrolyzed to reveal the carboxylic acid and remove the P₂ protecting group to reveal the amino group giving compounds of the formula 7. Cyclization of compounds of the formula 7 is accomplished by means of typical peptide coupling reagents, of which dicyclohexylcarbodiimide with dichloromethane as solvent is preferred. Compounds of the formula 8 thus prepared can be deprotected under acidic conditions to reveal the hydroxy group giving compounds 9. Oxidation of a compound of the formula 9 with Dess-Martin Periodinane or another suitable oxidizing agent such as IBX (o-iodoxybenzoic acid), oxalyl chloride in dimethyl sulfoxide (DMSO) (Swern oxidation) or PCC (pyridinium chlorochromate) to form the corresponding aldehyde of formula 10. This reaction may be carried out in dichloromethane (CH₂Cl₂), tetrahydrofuran (THF), dimethyl sulfoxide (DMSO) or a combination of two or more of these solvents. Reductive amination of a G-substituted piperidine or piperizine, as shown in Scheme A, using methods well known to those of skill in the art, with a compound of formula 10 yields the corresponding compound of formula 1A. The reductive amination can be performed, for example, utilizing catalytic hydrogenation methods or using a hydride reducing agent such as sodium triacetoxyborohydride or sodium cyanoborohydride. The reaction solvent can be 1,2-dichloroethane, tetrahydrofuran, acetonitrile, dimethylformamide or a combination of two or more of these solvents, with the optional addition of 1-10 equivalents of acetic acid. When the piperazine or piperidine hydrochloride or hydrobromide salt is used, a base such as triethylamine is typically added. The reductive amination is preferably conducted under conditions of neutral pH.

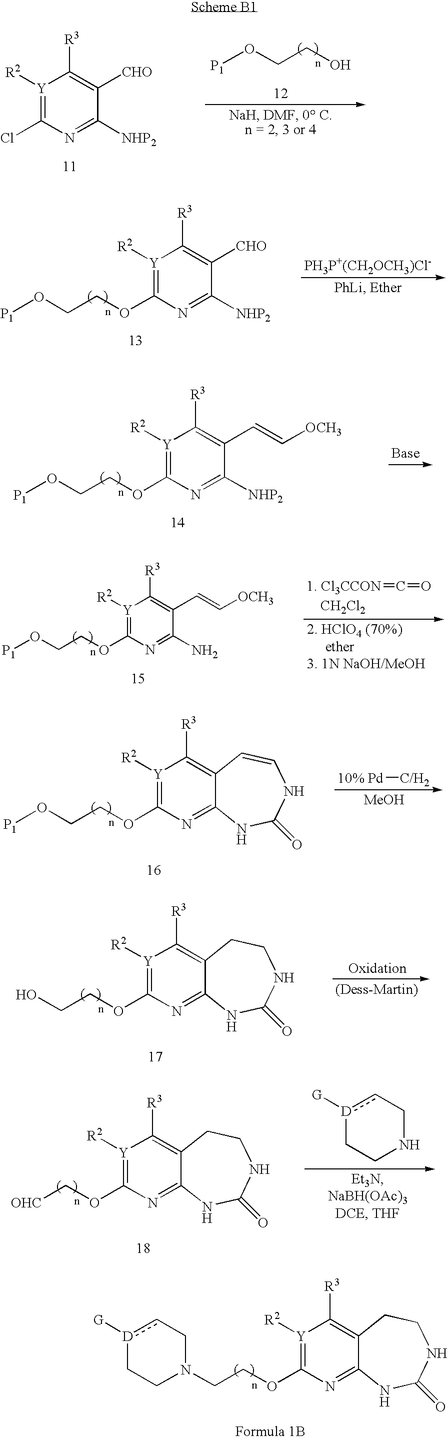

Scheme B1 illustrates a method for preparing compounds of formula 1B, i.e., compounds of formula 1 wherein A is —(CH₂)ₙO—, Q is N and Z is N. An aldehyde of the formula 11 such as N-(6-Chloro-3-formyl-pyridin-2-yl)-2,2-dimethyl-propionamide (Journal of Organic Chemistry, 55(15), 4744-50; 1990) where P₂ is an acyl protecting group such as pivaloyl which is removable under protic acidic or basic conditions can be reacted with a protected diol of the formula 12 where n is 2, 3 or 4 and P₁ is a hydroxy-protecting group such as benzyl which is removable under conditions of catalytic hydrogenation. Formation of an alkoxides of the formula 12 in an aprotic solvent followed by addition to a compound of the formula 11 give compounds of the formula 13. Specifically the reaction requires a base such as potassium tert-butoxide, sodium tert-butoxide, sodium hydride, potassium hydride, lithium diisopropylamide, lithium bis(trimethylsilyl)amide, potassium bis(trimethylsilyl)amide, or sodium bis(trimethylsilyl)amide. The solvents used may be THF, dioxane, ethylene glycol dimethylether, DMF, NMP, or DMSO or a combination of two or more of these solvents. The temperature of the reaction may vary from about 0° C. to 100° C. A compound of formula 13 is then reacted with (methoxymethyl)triphenylphosphonium chloride under Wittig or Horner-Emmons conditions giving a 3(2-Methoxy-vinyl) pyridine of the formula 14. Hydrolysis of the protecting group P2 under protic basic conditions exposes the amino group giving intermediates of the formula 15. Reaction of compounds of formula 15 with a reagent such as trichloroacetyl isocyanate provides urea intermediates, which upon acidification give trichloroacetylated seven membered ring intermediates. This acidification may be performed with 70% HClO₄ in aprotic solvents such as ethers or tetrahydrofuran. Treatment with a protic base or treatment in an alcohol such as ethanol or methanol liberates a pyridyl-fused-1,3-Dihydro-[1,3]diazepin-2-one of the formula 16. Catalytic hydrogenation of compounds of the formula 16 provides compounds of the formula described for the pyridyl-fused-1,3,4,5-tetrahydro-[1,3]diazepin-2-one 17 wherein P₁ is cleaved to expose the hydroxyl group. For example, the hydrogenation can be conducted using 5% to 20% palladium on activated carbon in a solvent such as methanol, ethanol, tetrahydrofuran, acetic acid, dimethylformamide, or a combination of two or more of these solvents for a period of about 5 hours to about 48 hours, preferably for about 24 hours, under a hydrogen pressure from about 1 to about 5 atmosphere, preferably about 1 atmosphere. Oxidization of the hydroxyl group to give aldehydes of the formula 18 can be performed as previously described above. These aldehydes are subsequently coupled to the piperazine or piperidine hydrochloride or hydrobromide salts of the formula 11 by reductive amination utilizing catalytic hydrogenation methods or by using a hydride reducing agent such as sodium triacetoxyborohydride previously described, giving the exemplified compounds of the formula 1B.

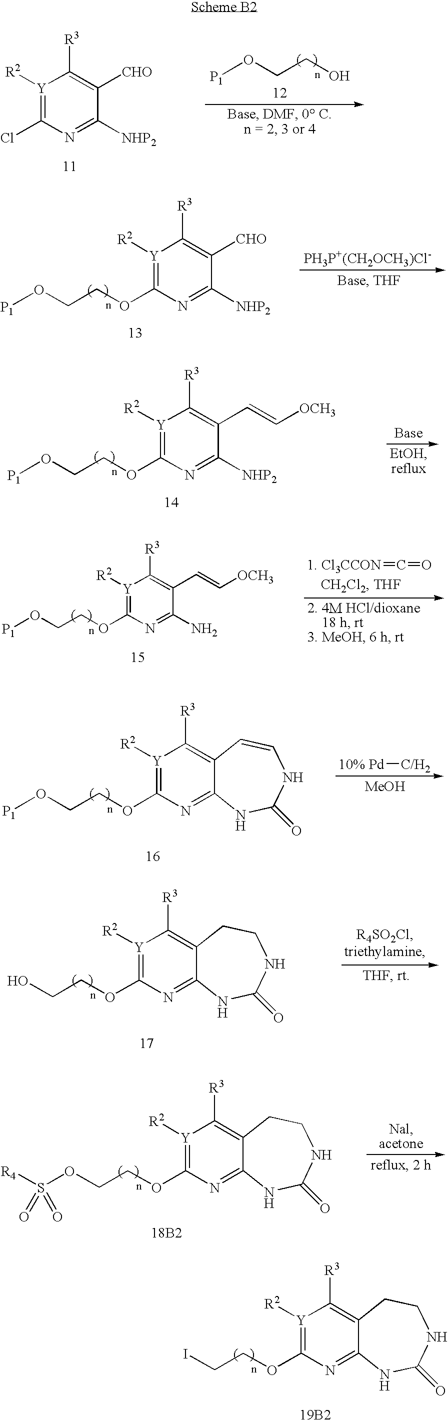

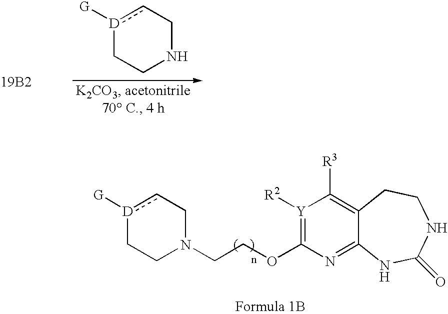

Scheme B2 illustrates alternate methods and conditions for preparing compounds of formula 1B, i.e., compounds of formula 1 wherein A is —(CH₂)ₙO—, Q is N and Z is N. An aldehyde of the formula 11, such as N-(6-Chloro-3-formyl-pyridin-2-yl)-2,2-dimethyl-propionamide (Journal of Organic Chemistry, 55(15), 4744-50; 1990) where P₂ is an acyl protecting group such as pivaloyl which is removable under protic acidic or basic conditions, can be reacted with a protected diol of the formula 12 where n is 2, 3 or 4 and P₁ is a hydroxy-protecting group such as benzyl which is removable under conditions of catalytic hydrogenation. Formation of alkoxides of the formula 12 in an aprotic solvent followed by addition to a compound of the formula 11 gives compounds of the formula 13. Specifically, the reaction requires a base such as potassium tert-butoxide, sodium tert-butoxide, sodium hydride, potassium hydride, lithium diisopropylamide, lithium bis(trimethylsilyl)amide, potassium bis(trimethylsilyl)-amide, or sodium bis(trimethylsilyl)amide. The solvents may be THF, dioxane, dimethylether, DMF, NMP, or DMSO or a combination of two or more of these solvents. The temperature of the reaction may vary from about 0° C. to 100° C. A compound of formula 13 is then reacted with (methoxymethyl)-triphenylphosphonium chloride under Wittig or Horner-Emmons conditions giving a 3(2-methoxy-vinyl)pyridine of the formula 14. Specifically, the reaction requires a base such as potassium tert-butoxide, sodium tert-butoxide, or phenyllithium in an aprotic solvent such as ethyl ether, dioxane or tetrahydrofuran. Removal of the protecting group P2 under protic basic conditions exposes the amino group. Specifically the reaction requires a base such as potassium or sodium hydroxide, in a protic solvent such as methyl or ethyl alcohol or mixtures of water and alcohol at elevated temperatures ranging from about 0° C. to 100° C. From the amino group of compounds of the formula 14 is formed a urea by reaction with a reagent such as trichloroacetyl isocyanate. Aprotic solvents including halocarbons, toluene or ethers such as dioxane or tetrahydrofuran may be used for this reaction. Acidification of this intermediate without isolationgives a trichloroacetylated seven-membered ring intermediate. The acidification may be performed by addition of ethereal or alcoholic solutions of mineral acids such as HCl in methanol or dioxane. Neutralization with a suitable base, such as sodium or potassium hydroxide in an alcohol such as ethanol or methanol, liberates a pyridyl-fused-1,3-Dihydro-[1,3]diazepin-2-one of the formula 16. Catalytic hydrogenation of compounds of the formula 16 provides compounds of the formula described for the pyridyl-fused-1,3,4,5-tetrahydro-[1,3]diazepin-2-one 17 wherein P₁ is cleaved to expose the hydroxyl group. For example, the hydrogenation can be conducted using 5 to 20% palladium on activated carbon in a solvent such as methanol, ethanol, tetrahydrofuran, acetic acid, dimethylformamide, or a combination of two or more of these solvents for a period of about 1 hour to about 48 hours, preferably for about 3 hours, under a hydrogen pressure from about 1 to about 5 atmospheres, preferably about 50 to 100 psig. Reaction of compounds of the formula 17 to give sulfonates of the formula 18B2 is performed by addition of sulfonyl chlorides or anhydrides wherin R₄ above is trifluoromethyl, methyl or tolyl in inert apolar solvents such as ethers, halocarbons or tetrahydrofuran in the presence of organic bases preferably triethyl amine. The sulfonates of formula 18B2 are subsequently reacted with sodium iodide in inert solvents such as acetone while at reflux to give intermediates of the formula 19B2. Reaction of the piperazine or piperidine hydrochloride or hydrobromide salts of the formula 11 with compounds of formula 19B2 can be performed in a suitable solvent such as tetrahydrofuran, dioxane, dimethylether, DMF, NMP, or DMSO, acetone or acetonitrile or a combination of two or more of these solvents. The reaction requires the presence of an organic or inorganic base, preferably triethylamine, sodium or potassium carbonate at elevated temperatures from 50° C. to 120° C., to give compounds of the formula 1B.

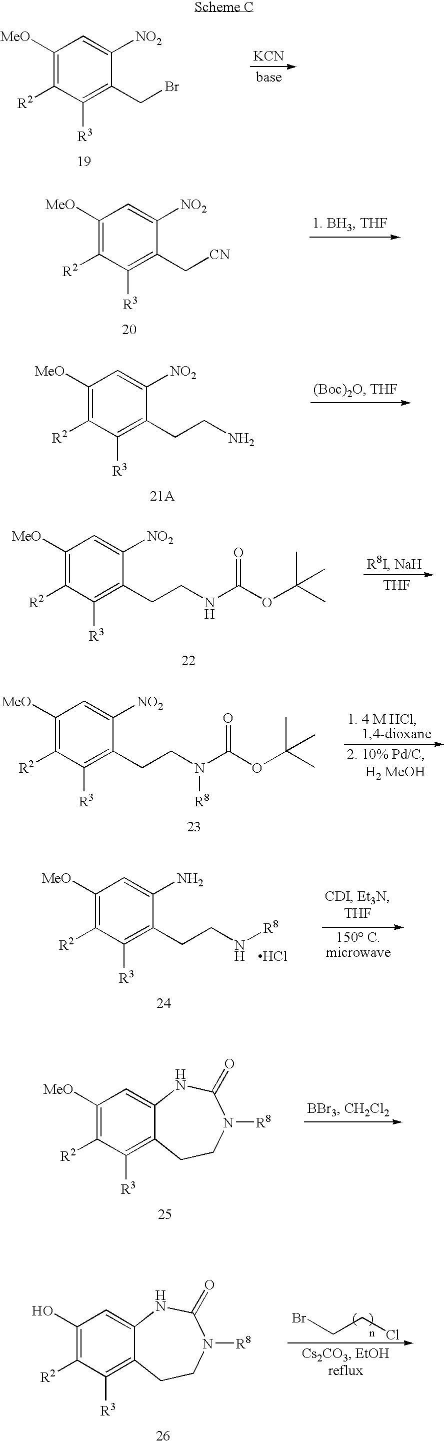

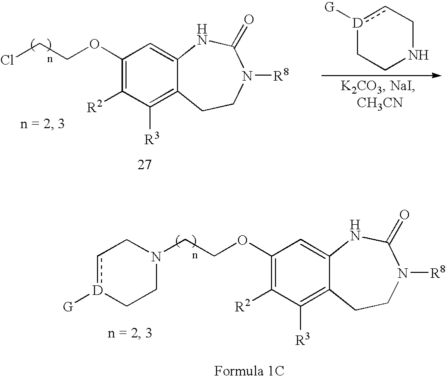

Scheme C illustrates a method for preparing compounds of formula 1C, i.e., compounds of formula 1 wherein A is —(CH₂)ₙO—, Q and Y are C, Z is N, and R⁸ is a (C₁-C₄) alkyl. Compounds of the formula 19 (Journal of Organic Chemistry, 4(7), 1238-46; 1984) are reacted with sodium or potassium cyanide to give nitriles of formula 20. Mild and selective chemical reducing reagents such as borane-tetrahydrofuran complex reduces the nitrile to give the amine of formula 21A. The free amine of formula 21A can be protected by an acyl protecting group 22, prior to deprotonation with a strong base and addition of a suitable alkyl halide, to produce compounds of formula 23. Sequential removal of the amino protecting group followed by reduction of the nitro group gives diamine compounds of formula 24. These can be cyclized by reaction with a suitable activated carbonyl such as phosgene or carbonyldiimidazole to give compounds of formula 25. Removal of the methoxy group by methods well known to those skilled in the art gives phenols of formula 26. Preferable reagents for this process include boron tribromide in dichloromethane. The phenols thus prepared can be reacted with an excess of 1 to 5 equivalents of an appropriate alkyl dihalide. The reaction may be run in solvents that include singly or as mixtures, water, acetonitrile, acetone, DMF, DME, or ethanol and a variety of bases including sodium, potassium or cesium carbonate, sodium or potassium hydroxide, at temperatures ranging from 50 to 140° C. The resulting compounds of formula 27 are then reacted with a G-substituted piperazine or piperidine, as depicted in Scheme C, to yield the desired compound of formula 1C. This reaction is preferably run in the presence of a base such as potassium carbonate, sodium carbonate, cesium carbonate, triethylamine or diisopropylethylamine. The solvent used may be acetonitrile, water, tetrahydrofuran, dioxane, acetone, methyl isobutyl ketone, benzene or toluene, or a combination of two or more of these solvents. Inorganic salts such as sodium or potassium iodide may be employed as catalysts in the reaction. The temperature of the reaction may vary from about ambient temperature to about the reflux temperature of the solvent used. The reaction may also be heated by microwave irradiation.

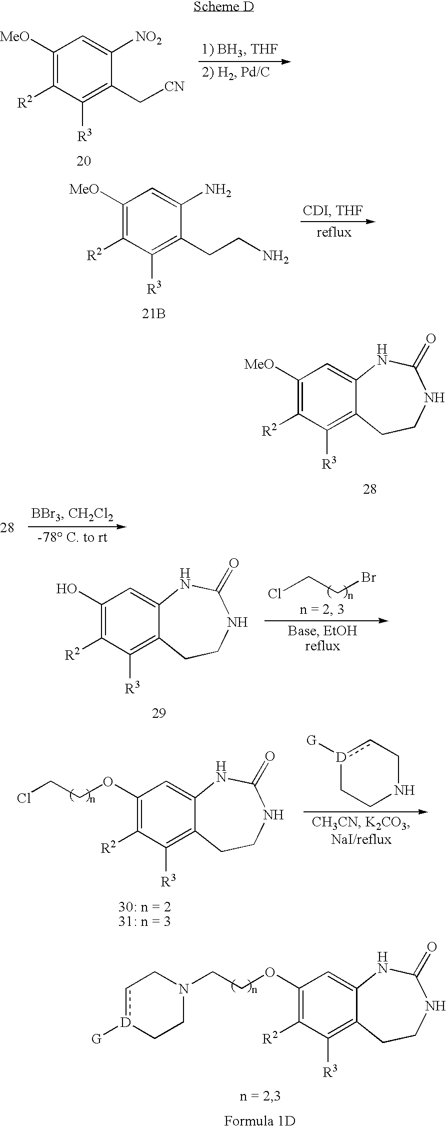

Scheme D illustrates a method for preparing compounds of formula 1D, i.e., compounds of formula 1 wherein A is —(CH₂)ₙO—, Q and Y are C, Z is N and R⁸ is H. By close analogy to Scheme C, compounds of the formula 20 (also known in the prior art: Journal of Heterocyclic Chemistry, 41(3), 317-326; 2004) are reduced singly or by a combination of reagents such as borane-tetrahydrofuran complex followed by a catalytic hydrogenation which reduces both the nitrile and the nitro group to give diamines of the formula 21B. These can be cyclized by reaction with a suitable source of activated carbonyl such as phosgene or carbonyldiimidazole to give compounds of the formula 28. Removal of the methoxy group by methods well known to those skilled in the art and previously described gives phenols of the formula 29. Alkylation of such phenols give compounds of the formulas 30 and 31, which are subsequently reacted with piperazines or piperidines to give compounds of the formula 1C in a manner similar those analogous procedures previously described.

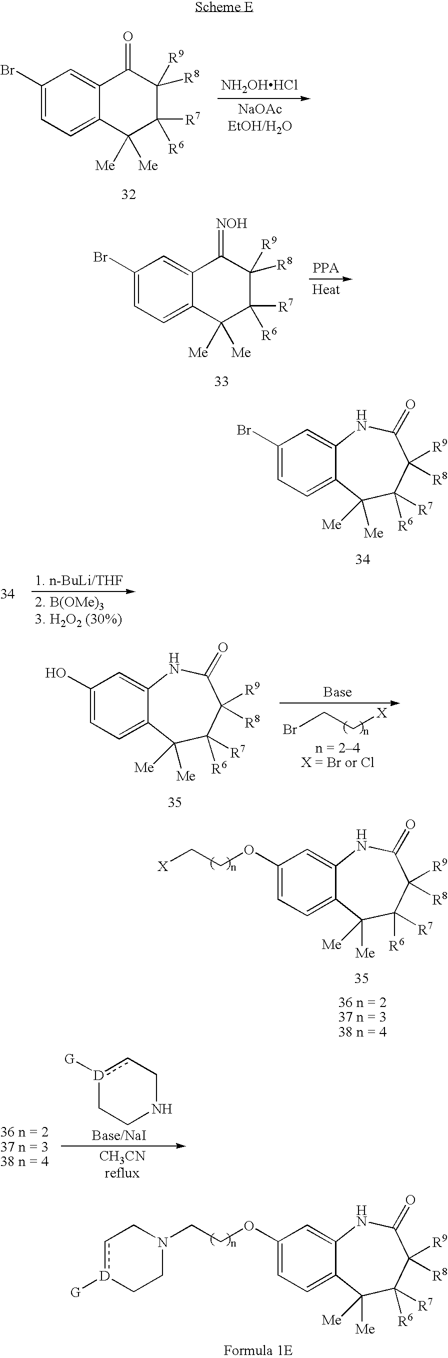

Scheme E illustrates a method for preparing compounds of formula 1E, i.e., compounds of formula 1 wherein A is —(CH₂)ₙO—, Q and Z is C, and both R⁴ and R⁵ are methyl (Me). A substituted tetralone of formula 32 (Tetrahedron Letters, 37(12), 1941-1; 1996) above can be converted to an oxime of the formula 33 and rearranged by methods known to those skilled in the art (Schmidt rearrangement, Synthetic Communications, 30(19), 3481-3490) to give the corresponding 1,3,4,5-Tetrahydro-benzo[b]azepin-2-ones of formula 34. Conversion of aryl bromides to phenols by lithium exchange followed by reaction with a boronate subsequently followed by oxidation provides phenols of the formula 35. The conditions for the reaction of the phenols with appropriate alkyl dihalides, followed by displacements with G-substituted piperazines or piperidines to give compounds of the formula 1E has been previously described based upon analogy to conditions for compounds of the formula 1D above.

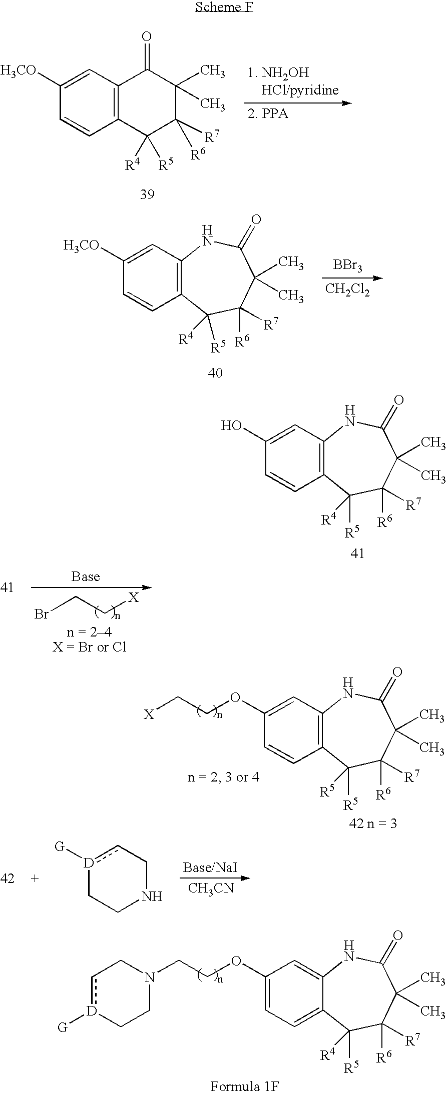

Scheme F illustrates a method for preparing compounds of formula 1F, i.e., compounds of formula 1 wherein A is —(CH₂)ₙO—, Q and Z is C, and both R⁸ and R⁹ are Me. By close analogy to chemistry previously described in Scheme E, an alternately substituted tetralone of the formula 39 above can be converted to an oxime and rearranged to the corresponding 1,3,4,5-Tetrahydro-benzo[b]azepin-2-ones of formula 40. Conversion of aryl methoxy groups to the corresponding phenols is described previously in Scheme D above. These processes give compounds of the formula 41 which are then converted by reactions of the phenols with appropriate alkyl dihalides (42), followed by displacements with G-substituted piperazines or piperidines to give compounds of the formula 1F.

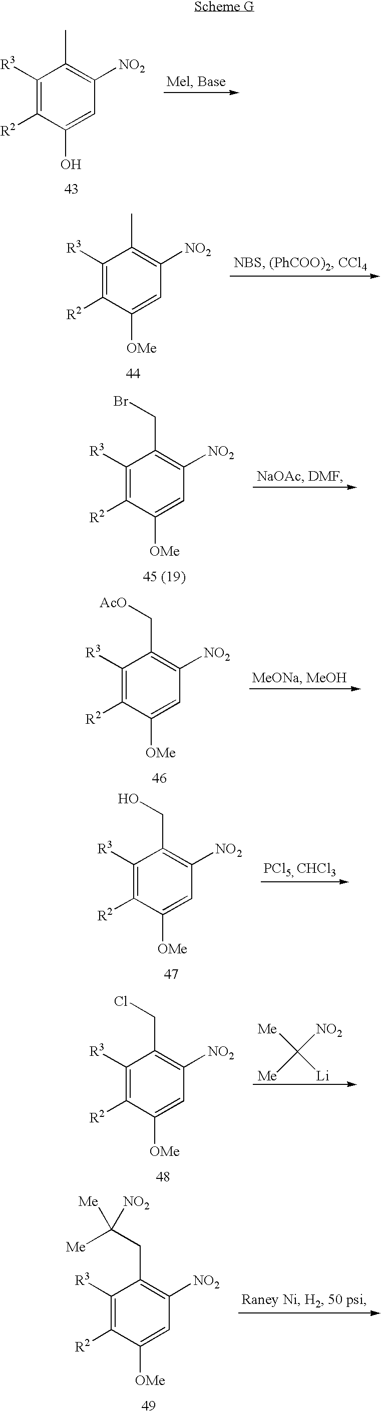

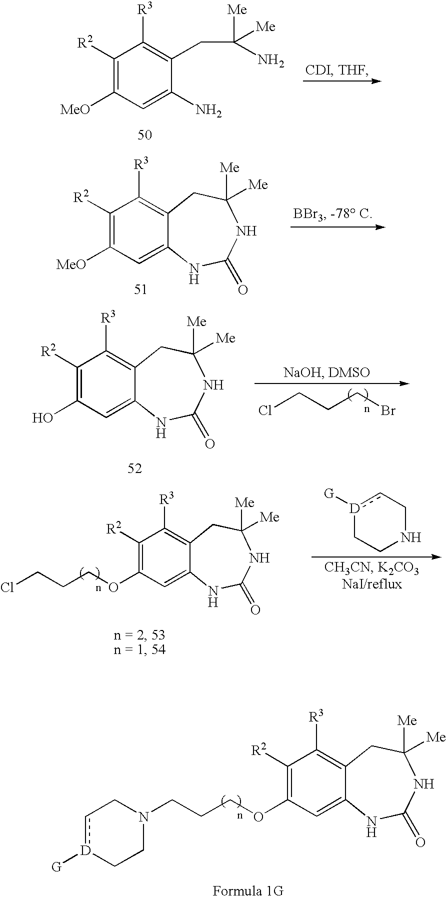

Scheme G illustrates a method for preparing compounds of formula 1G, i.e., compounds of formula 1 wherein A is —(CH₂)ₙO—, Q and Y are C, Z is N, R⁴ and R⁵ are H, R⁶ and R⁷ are both Me, and R⁸ is H. With the provision that R³ and R⁴ are not groups susceptible to halogenation, phenolic compounds of the formula 43 are converted to the corresponding methoxy compounds of the formula 44 in a manner well known to those skilled in the art. Similarly halogenation to give compounds of the formula 45 using n-bromo or n-chlorosuccinimide is well known. Compounds of the formula 46 are obtained by displacement of the halo group by sodium acetate, followed by basic hydrolysis to give compounds of the formula 47. The benzyl hydroxyl group thus formed may be converted to a leaving group, of which in this case chloro is preferred, to give compounds of the formula 48. Deprotonation of a nitro alkane such as 2-nitropropane provides a reagent which can replace the leaving group of compounds of the formula 48 with a dialkylnitro functionality to give a compound of the formula 49. Catalytic hydrogenation of compounds of the formula 49 by any of a variety of methods known to those skilled in the art gives diamines of the formula 50. As previously described above, compounds of the formula 50 can be cyclized by reaction with a suitable source of activated carbonyl such as phosgene or carbonyldiimidazole to give compounds of the formula 51. Removal of the methoxy group by methods well known to those skilled in the art and previously described gives phenols of the formula 52. Alkylation of such phenols give compounds of the formulas 53 and 54, which are subsequently reacted with piperazines or piperidines to give compounds of the formula 1G in a manner similar those analogous procedures previously described.

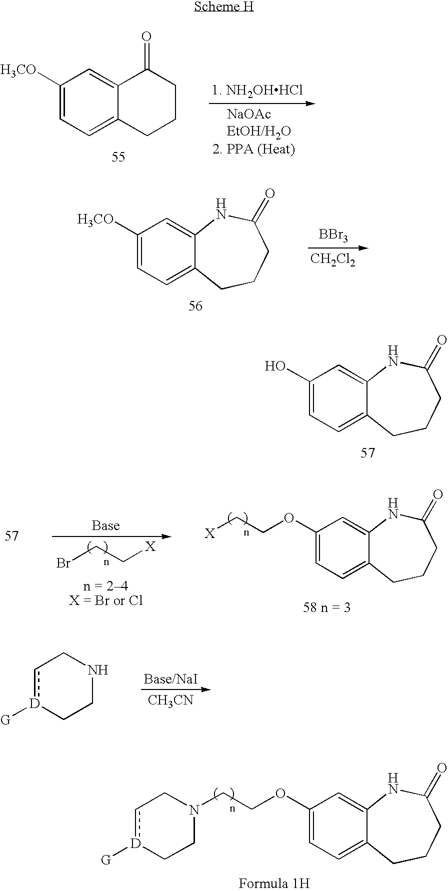

Scheme H illustrates a method for preparing compounds of formula 1H, i.e., compounds of formula 1 wherein A is —(CH₂)ₙO—, Q and Z is C, and each of R⁴ to R⁹ is H. By close analogy to chemistry previously described in Scheme E, an alternately substituted tetralone of the formula 55 above can be converted to an oxime and rearranged to the corresponding 1,3,4,5-Tetrahydro-benzo[b]azepin-2-ones of formula 56. Conversion of aryl methoxy groups to the corresponding phenols is described previously in Scheme D above. These processes give compounds of the formula 57 which are then converted by reactions of the phenols with appropriate alkyl dihalides to give compounds of the formula 58, followed by displacements with G-substituted piperazines or piperidines to give compounds of the formula 1H.

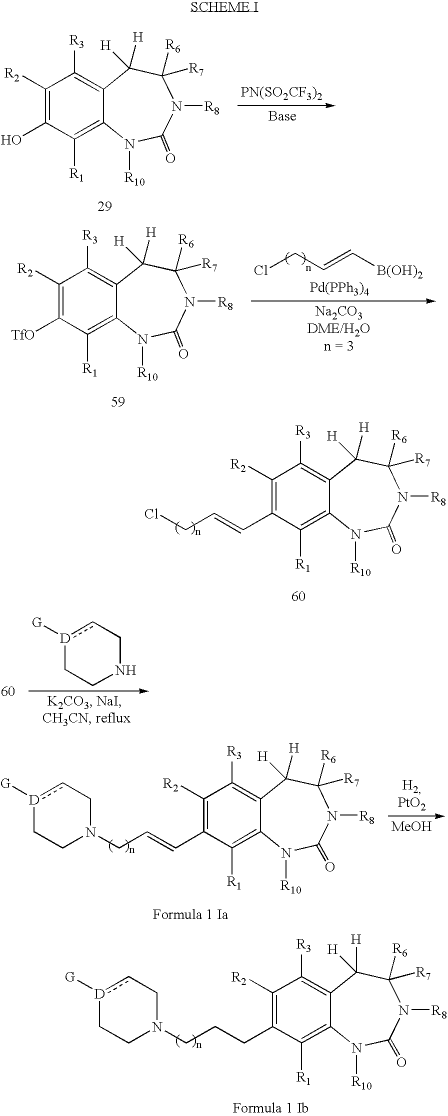

Scheme I illustrates a method for preparing compounds of formulae 1 Ia and 1 Ib, i.e., compounds of formula 1 wherein A is —(CH₂)ₙCH₂—, Q and Y are C, Z is N, and both R⁴ and R⁵ are H. For example, compounds of the formula 59 formed from the triflation of compounds of the formula 29 can be reacted with a chloroalkenylboronic acid of the formula Cl(CH₂)ₙCH═CHB(OH)₂, wherein n is an integer from 1 to 3, under palladium-catalyzed Suzuki cross-coupling conditions (Chem. Rev. 1995, 95, 2457), to give the corresponding compounds of formula 60. For example, the coupling can be conducted using a catalytic amount of tetrakis(triphenylphosphine)-palladium(0) in the presence of a base such as aqueous sodium carbonate, sodium hydroxide, or sodium ethoxide, in a solvent such as THF, dioxane, ethylene glycol dimethylether, ethanol (EtOH) or benzene. The temperature of the reaction may vary from about ambient temperature to about the reflux temperature of the solvent used. The resulting compounds of the formula 60 are then reacted with a G-substituted piperazine or piperidine, as depicted in Scheme I, to yield the corresponding compounds of formula 1 Ia. This reaction is typically run in the presence of a base such as potassium carbonate, sodium carbonate, cesium carbonate, triethylamine or diisopropylethylamine. Typical solvents include acetonitrile, water, THF, dioxane, acetone, methyl isobutyl ketone, benzene or toluene, or a combination of two or more of these solvents. Inorganic salts such as sodium or potassium iodide may be employed as catalysts in the reaction. The temperature of the reaction can range from about ambient temperature to about the reflux temperature of the solvent. The reaction may also be conducted under microwave irradiation. Hydrogenation of compounds of the formula 1 Ia, using methods well known to those of skill in the art, yields the desired compounds of formula 1 Ib. For example, the hydrogenation reaction can be conducted using catalytic PtO₂ or Raney-nickel in a solvent such as ethanol, methanol, or THF, or a combination of two or more of these solvents, at a hydrogen pressure from about 1 atmosphere to about 5 atmospheres.

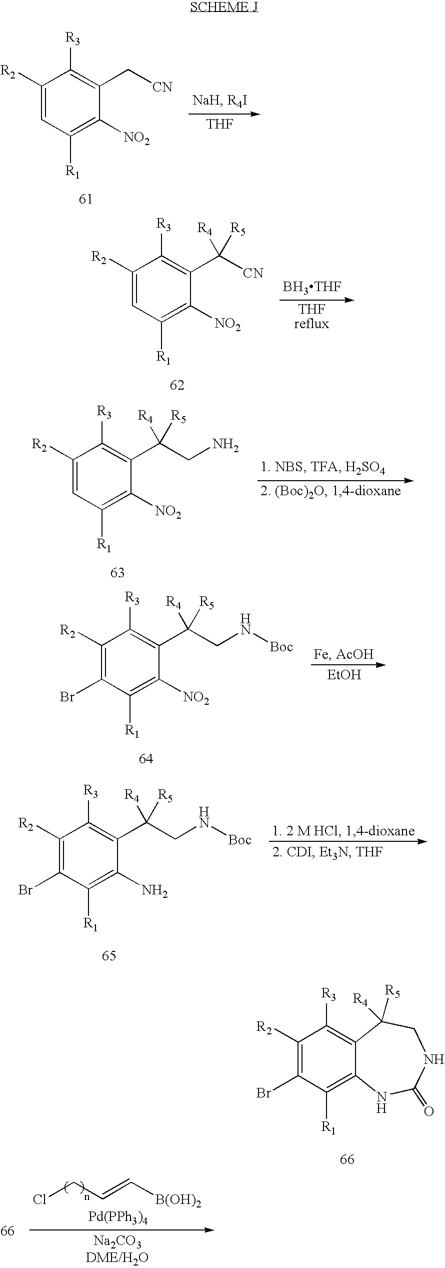

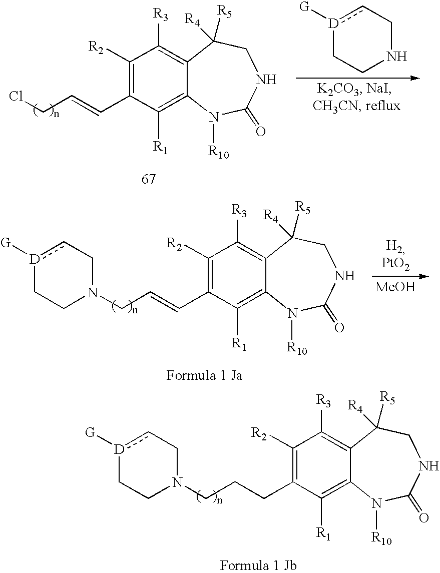

Scheme J illustrates a method for preparing compounds of formulae 1 Ja and 1 Jb, i.e., compounds of formula 1 wherein A is —(CH₂)ₙCH₂—, Q and Y are C, Z is N and R₃, R₄ are methyl. Compounds of the formula 61 are deprotonated by suitable bases and alkylated to install the substitutions R⁴ and R⁵ and give compounds of the formula 62. By analogy, the reactions previously described for reduction of compounds of the formula 20 to 21A, N-protection to 22, reduction of the nitro to 23, deprotection of N to 24, and cyclization to 25 in Scheme C can be applied to compounds of the formulas 61 thru 66 above, respectively. Conditions for reaction of compounds of the formula 66 with a chloroalkenylboronic acid of the formula Cl(CH₂)ₙCH═CHB(OH)₂ to give compounds of the formula 67 have previously been described in Scheme I above. Also in like manner, the procedures for reaction of compounds of the formula 67 with a G-substituted piperazines or piperidines, to give compounds of the formulas 1Ja are previously described. Hydrogenation of compounds of the formula 1Ja, using methods mentioned above, well known to those of skill in the art, yields the desired compounds of formula 1 Jb.

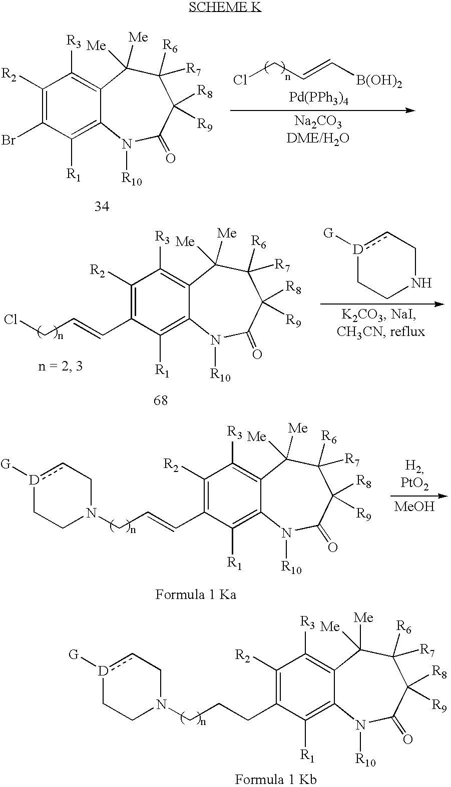

Scheme K illustrates a method for preparing compounds of formulae 1 Ka and 1 Kb, i.e., compounds of formula 1 wherein A is —(CH₂)ₙCH₂—, Q, Y and Z are each C, and R⁴ and R⁵ are Me. Conditions for preparations of compounds of the formulas 68, 1Ka and 1Kb are all analogous to those in Scheme J from 66 thru 1Jb.

The preparation of other compounds of the formula 1 not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of the formula 1 and the intermediates shown in the above reaction schemes can be isolated and purified by conventional procedures, such as recrystallization or chromatographic separation.

The preparation of other compounds of the formula 1 not specifically described in the foregoing experimental section can be accomplished using combinations of the reactions described above that will be apparent to those skilled in the art.

In each of the reactions discussed or illustrated above, pressure is not critical unless otherwise indicated. Pressures from about 0.5 atmospheres to about 5 atmospheres are generally acceptable, and ambient pressure, i.e., about 1 atmosphere, is preferred as a matter of convenience.

The compounds of the formula 1 and the intermediates shown in the above reaction schemes can be isolated and purified by conventional procedures, such as recrystallization or chromatographic separation.

The compounds of the formula 1 and their pharmaceutically acceptable salts, can be administered to mammals via either the oral, parenteral (such as subcutaneous, intravenous, intramuscular, intrasternal and infusion techniques), rectal, buccal or intranasal routes. In general, these compounds are administered in doses ranging from about 3 mg to about 600 mg per day, in single or divided doses (i.e., from 1 to 4 doses per day), although variations will necessarily occur depending upon the species, weight and condition of the patient being treated and the patient's individual response to said medicament, as well as on the type of pharmaceutical formulation chosen and the time period and interval at which such administration is carried out. However, a dosage level that is in the range of about 10 mg to about 100 mg per day is most desirably employed. In some instances, dosage levels below the lower limit of the aforesaid range may be more than adequate, while in other cases still larger doses may be employed without causing any harmful side effects, provided that such higher dose levels are first divided into several small doses for administration throughout the day. The specific amount of a compound of formula 1, or a pharmaceutically acceptable salt thereof, that may be administered to a mammal for treating a disorder or condition may vary depending on a number of factors, such as the method of use, age and sex of the patient, the specific disorder or condition being treated, or conditions of the symptoms of the disorder or condition being treated, and can be readily determined by a person skilled in the art.

The novel compounds of the present invention may be administered alone or in combination with pharmaceutically acceptable carriers or diluents by any of the routes previously indicated, and such administration may be carried out in single or multiple doses. More particularly, the novel therapeutic agents of this invention can be administered in a wide variety of different dosage forms, i.e., they may be combined with various pharmaceutically acceptable inert carriers in the form of tablets, capsules, lozenges, troches, hard candies, suppositories, jellies, gels, pastes, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the weight ratio of the novel compounds of this invention to the pharmaceutically acceptable carrier will be in the range from about 1:6 to about 2:1, and preferably from about 1:4 to about 1:1.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art.

This invention also relates to methods of treating anxiety, depression, schizophrenia and the other disorders referred to in the description of the methods of the present invention, wherein a novel compound of this invention and one or more of the other active agents referred to above (e.g., an NK1 receptor antagonist, tricyclic antidepressant, 5HT1D receptor antagonist, or serotonin reuptake inhibitor) are administered together, as part of the same pharmaceutical composition, as well as to methods in which such active agents are administered separately as part of an appropriate dose regimen designed to obtain the benefits of the combination therapy. The appropriate dose regimen, the amount of each dose of an active agent administered, and the specific intervals between doses of each active agent will depend upon the subject being treated, the specific active agent being administered and the nature and severity of the specific disorder or condition being treated. In general, the novel compounds of this invention, when used as a single active agent or in combination with another active agent, will be administered to an adult human in an amount from about 1 mg to about 300 mg per day, in single or divided doses, preferably from about 1 to about 150 mg per day. Such compounds may be administered on a regimen of up to 6 times per day, preferably 1 to 4 times per day, especially 2 times per day and most especially once daily. Variations may nevertheless occur depending upon the species of animal being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effect, provided that such larger doses are first divided into several small doses for administration throughout the day.

A proposed daily dose of a 5HT reuptake inhibitor, preferably sertraline, in the combination methods and compositions of this invention, for oral, parenteral or buccal administration to the average adult human for the treatment of the conditions referred to above, is from about 0.1 mg to about 2000 mg, preferably from about 1 mg to about 200 mg of the 5HT reuptake inhibitor per unit dose, which could be administered, for example, 1 to 4 times per day. A proposed daily dose of a 5HT1D receptor antagonist in the combination methods and compositions of this invention, for oral, parenteral, rectal or buccal administration to the average adult human for the treatment of the conditions referred to above, is from about 0.01 mg to about 2000 mg, preferably from about 0.1 mg to about 200 mg of the 5HT1D receptor antagonist per unit dose, which could be administered, for example, 1 to 4 times per day.

For intranasal administration or administration by inhalation, the novel compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch. Formulations of the active compounds of this invention for treatment of the conditions referred to above in the average adult human are preferably arranged so that each metered dose or “puff” of aerosol contains 20 μg to 1000 μg of active compound. The overall daily dose with an aerosol will be within the range 100 μg to 10 mg. Administration may be several times daily, for example 2, 3, 4 or 8 times, giving for example, 1, 2 or 3 doses each time.

The ability of the novel compounds of this invention to bind to the dopamine D₂ receptor can be determined using conventional radioligand receptor binding assays. All receptors can be heterologously expressed in cell lines and binding assays can be conducted in membrane preparations from the cell lines using procedures outlined below. IC₅₀ concentrations can be determined by nonlinear regression of concentration-dependent reduction in specific binding. The Cheng-Prussoff equation can be used to convert the IC₅₀ to Ki concentrations. See Example 71, below, for a description of the assay used to determine the binding of the compounds of this invention to the dopamine D₂ receptor, and the binding data obtained for the assayed compounds.

Compounds of the present invention preferably exhibit Ki values of no more than 100 nM, more preferably no more than 50 nM, even more preferably no more than 25 nM, most preferably no more than 10 nM.

### EXAMPLES

The following examples are provided for the sole purpose of illustrating one or more of the embodiments of the invention described above and should be construed as limiting the scope of the invention. Among these Examples, Examples 1-70 are provied to illustrate the preparation of several compounds of the present invention. Melting points are uncorrected. NMR data are reported in parts per million and are referenced to the deuterium lock signal from the sample solvent. Any reference to a “title compound” in an example, below, refers to the compound named in the title of that particular example. Examples 71 and 72 are provided to illustrate some of the pharmacological properties of several compounds of the invention.

### Example 1

### 3-(Triphenyl-λ⁵-phosphanylidene)-propionic acid bromide (2)

To a solution of 3-Bromo-propionic acid (1) (15 g, 98 mmol) in acetonitrile (MeCN) (200 mL) was added triphenylphosphine (Ph₃P) (25.71 g, 98 mmol) and refluxed for 24 h. The solvent was evaporated, the resulting orange color oil was washed with diethylether (Et₂O), and the mother liquor was co-concentrated with toluene to obtain the title compound (2) as white solid. ¹H-NMR (400 MHz, CDCl₃) δ 7.84-7.68 (m, 15H), 3.77 (m, 2H), 3.08 (m, 2H).

### N-{3-Formyl-6-[4-(tetrahydro-pyran-2-yloxy)-butoxy]-pyridin-2-yl}-2,2-dimethyl-propionamide (4)

To a solution of 2,2-Dimethyl-N-{6-[4-(tetrahydro-pyran-2-yloxy)-butoxy]-pyridin-2-yl}-propionamide (3) (US Pat App Pub No 20050043309) (10 g, 28.53 mmol) in dry tetrahydrofuran (THF) (120 mL) was added n-butyllithium (n-BuLi) (2.5 M in hexanes, 28.53 mL, 71.33 mmol) at −78° C. The reaction mixture was stirred for 3 h at 0° C. N,N-dimethylformamide (DMF) (6.6 mL, 85.6 mmol) was added to the reaction mixture at −78° C., and was stirred for 2 h at room temperature. Saturated NaHCO₃ solution was added and extracted with EtOAc. The organic layer was washed with H₂O, brine, and dried over Na₂SO₄. Evaporation under vacuum yielded the title compound (4) as an oil. ¹H-NMR (400 MHz, CDCl₃) δ 10.21 (s, 1H), 8.27 (d, 1H), 6.98 (d, 1H), 5.11 (m, 1H), 4.23-4.19 (m, 3H), 3.99-3.84 (m, 3H), 2.38-1.96 (m, 10H), 1.82 (s, 9H).

### 4-{2-(2,2-Dimethyl-propionylamino)-6-[4-(tetrahydro-pyran-2-yloxy)-butoxy]-pyridin-3-yl}-but-3-enoic acid (5)

NaH (0.95 g, 39.6 mmol) was added in portions to dimethyl sulfoxide (DMSO) (10 mL) at room temperature, followed by addition of more DMSO (5 mL). After being stirred for 10 min, 3-(Triphenyl-15-phosphanylidene)-propionic acid bromide (2) (8.22 g, 19.81 mmol) was added in portions. The reaction mixture was stirred until a light orange color of the phosphonium ylide was formed (aprox. 30 min). N-{3-Formyl-6-[4-(tetrahydro-pyran-2-yloxy)-butoxy]-pyridin-2-yl}-2,2-dimethyl-propionamide (4), 3 g, 7.92 mmol) pre-dissolved in THF (10 mL) was added dropwise. The reaction mixture was stirred overnight at room temperature. Ice was added to the reaction mixture and extracted with Et₂O (×3). The aq. layer was acidified to pH 6 with 3M HCl, and extracted with EtOAc (×3). The organic layer was washed with H₂O, brine, dried over Na₂SO₄ and evaporated to give the title compound (5) as an oil. ¹H-NMR (400 MHz, CDCl₃) δ 7.42 (d, 1H), 6.60 (d, 1H), 6.38 (m, 1H), 5.81 (m, 1H), 4.60 (m, 1H), 4.26 (t, 2H), 3.82 (m, 2H), 3.45 (m, 2H), 3.21 (m, 2H), 1.91-1.42 (m, 10H), 1.32 (s, 9H).

### 4-{2-(2,2-Dimethyl-propionylamino)-6-[4-(tetrahydro-pyran-2-yloxy)-butoxy]-pyridin-3-yl}-butyric acid (6)

To a solution of 4-{2-(2,2-Dimethyl-propionylamino)-6-[4-(tetrahydro-pyran-2-yloxy)-butoxy]-pyridin-3-yl}-but-3-enoic acid (5) (8.8 g, 20.27 mmol) in EtOH (80 mL) was added NaHCO₃ (8.51 g, 101.38 mmol), and N₂ gas was allowed to bubble for 15 min, Pd—C (35% v/v) was added in portions. The reaction mixture was stirred overnight under H₂ at atmospheric pressure, and filtered through celite. The filtrate was concentrated under vacuum to give the title compound (6) as a thick oil. ¹H-NMR (400 MHz, CDCl₃) δ 7.56 (s, 1H), 7.44 (d, 1H), 6.58 (d, 1H), 4.61 (m, 1H), 4.22 (t, 2H), 3.81 (m, 2H), 3.44 (m, 2H), 2.56 (t, 2H), 2.32 (t, 2H), 1.93-1.42 (m, 10H), 1.36 (s, 9H).

### 4-{2-Amino-6-[4-(tetrahydro-pyran-2-yloxy)-butoxy]-pyridin-3-yl}-butyric acid (7)

To a solution of 4-{2-(2,2-Dimethyl-propionylamino)-6-[4-(tetrahydro-pyran-2-yloxy)-butoxy]-pyridin-3-yl}-butyric acid (6) (7.2 g, 16.58 mmol) in EtOH (50 mL) was added 2.5M KOH (50 mL). The reaction mixture was refluxed for 36 h and cooled in an ice-bath and gradually acidified to pH 6 with 3M HCl when the title compound (7) crystallized as a white solid. ¹H-NMR (400 MHz, CDCl₃) δ 7.20 (d, 1H), 5.88 (d, 1H), 4.61 (m, 1H), 4.04 (t, 2H), 3.82 (m, 2H), 3.47 (m, 2H), 2.43 (m, 4H), 1.96-1.42 (m, 10H).

### 2-[4-(Tetrahydro-pyran-2-yloxy)-butoxy]-5,6,7,9-tetrahydro-pyrido[2,3-b]azepin-8-one (8)

To a solution of 4-{2-Amino-6-[4-(tetrahydro-pyran-2-yloxy)-butoxy]-pyridin-3-yl}-butyric acid (7) (2.71 g, 7.69 mmol) in CH₂Cl₂ (230 mL) was added dicyclohexylcarbodiimide (DCC) (2.78 g, 13.47 mmol) and 4-dimethylaminopyridine (DMAP) (1.64 g, 13.47 mmol), and stirred overnight at room temperature. The reaction mixture was cooled on in ice-bath and solid was filtered off. The filtrate was concentrated and column chromatography on silica gel, eluting with MeOH:CHCl₃ (3:97), gave the title compound (8) as a thick oil. ¹H-NMR (400 MHz, CDCl₃) δ 7.59 (s, 1H), 7.41 (d, 1H), 6.43 (d, 1H), 4.60 (m, 1H), 4.21 (t, 2H), 3.81 (m, 2H), 3.44 (m, 2H), 2.74 (t, 2H), 2.47 (t, 2H), 2.21 (m, 2H), 1.92-1.43 (m, 10H).

### 2-(4-Hydroxy-butoxy)-5,6,7,9-tetrahydro-pyrido[2,3-b]azepin-8-one (9)

To a solution of 2-[4-(Tetrahydro-pyran-2-yloxy)-butoxy]-5,6,7,9-tetrahydro-pyrido[2,3-b]azepin-8-one (8) (2.1 g, 6.28 mmol) in methanol (MeOH) (15 mL) was added 3M HCl (3.15 mL), and stirred for 4 h at room temperature. Sat. NaHCO₃ was added and extracted with ethyl acetate (EtOAc) (×3). The organic layer was washed with H₂O, brine, dried over Na₂SO₄, and evaporated. Purification of the resulting orange color oily material on silica column, eluting with MeOH:CHCl₃ (5:95), gave the title compound (9) as an oil. ¹H-NMR (400 MHz, CD₃OD) δ 7.55 (d, 1H), 6.51 (d, 1H), 4.24 (t, 2H), 3.60 (t, 2H), 2.70 (t, 2H), 2.37 (t, 2H), 2.21 (m, 2H), 1.82 (m, 2H), 1.66 (m, 2H).

### 4-(8-Oxo-6,7,8,9-tetrahydro-5H-pyrido[2,3-b]azepin-2-yloxy)-butyraldehyde (10)

To a mixture of 2-(4-Hydroxy-butoxy)-5,6,7,9-tetrahydro-pyrido[2,3-b]azepin-8-one, (0.68 g, 2.72 mmol) (9) in 1,2-dichloroethane (DCE) (55 mL) was added o-Iodoxybenzoic Acid (1.9 g, 6.8 mmol) and refluxed for 5 h. The reaction mixture was filtered and the filtrate was concentrated, and purified on silica column, eluting with EtOAc:hexanes (6:4) and then changing to (9:1), to give the title compound (10), as a white solid. ¹H-NMR (400 MHz, CDCl₃) δ 9.82 (s, 1H), 7.41 (d, 1H), 7.38 (s, 1H), 6.42 (d, 1H), 4.22 (t, 2H), 2.76 (t, 2H), 2.62 (t, 2H), 2.47 (t, 2H), 2.22 (m, 2H), 2.08 (m, 2H).

### Example 2

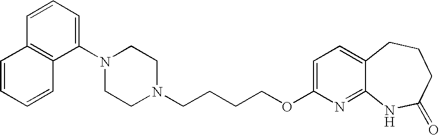

### 2-[4-(4-Naphthalen-1-yl-piperazin-1-yl)-butoxy]-5,6,7,9-tetrahydro-pyrido[2,3-b]azepin-8-one

To a solution of 4-(8-Oxo-6,7,8,9-tetrahydro-5H-pyrido[2,3-b]azepin-2-yloxy)-butyraldehyde (10), (0.15 g, 0.6 mmol) and 1-naphthylpiperazine (0.208 g, 0.84 mmol) in 1,2-dichloroethane (8 mL) at 0° C. was added triethylamine (Et₃N) (0.23 mL, 1.68 mmol). After stirring at room temperature for 10 min, NaBH(OAc)₃ (0.195 g, 0.92 mmol) was added to the reaction mixture and let it stirred for 1.5 h. Sat. NaHCO₃ solution (10 mL) was added to the reaction and stirred for 15 min, followed by the addition of EtOAc (30 mL). The organic layer was separated and washed with sat. NaHCO₃, brine, and dried over Na₂SO₄. Purification of the resulting brown oily material on silica column, eluting with EtOAc:MeOH (98:2), afforded 0.27 g of the coupled product as a white foam. The latter was dissolved in minimum amount of CH₂Cl₂ and 1M HCl in diethyl ether (0.6 mL, 0.6 mmol) at 0° C. was added dropwise. Addition of more diethyl ether at room temperature crystallized the title compound as a white solid, mp. 226-27° C. ¹H-NMR (400 MHz, DMSO-d₆) δ 9.72 (s, 1H), 8.16 (d, 1H), 7.92 (d, 1H), 7.66 (d, 1H), 7.61 (d, 1H), 7.56 (m, 2H), 7.21 (d, 1H), 6.56 (d, 1H), 4.22 (t, 2H), 3.62 (m, 2H), 3.56-3.24 (m, 8H), 3.16 (m, 2H), 2.62 (t, 2H), 2.21 (t, 2H), 1.96-1.78 (m, 4H).

### Example 3

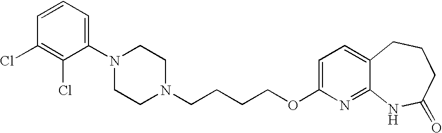

### 2-{4-[4-(2,3-dichloro-phenyl)-piperaxin-1-yl]-butoxy}-5,6,7,9-tetrahydro-pyrido[2,3-b]azepin-8-one

2-{4-[4-(2,3-dichloro-phenyl)-piperaxin-1-yl]-butoxy}-5,6,7,9-tetrahydro-pyrido[2,3-b]azepin-8-one was produced using a process similar to Example 2, wherein 1-(2,3-dichloro-phenyl)-piperazine hydrochloride (Lancaster) was substituted for 1-naphthylpiperazine in the first step of the process. The title compound crystallized in the final step as a white solid, mp. 188-89° C. ¹H-NMR (400 MHz, DMSO-d₆) δ 9.73 (s, 1H), 7.61 (d, 1H), 7.39 (m, 1H), 7.22 (d, 1H), 6.56 (d, 1H), 4.22 (t, 2H), 3.59 (m, 2H), 3.42-3.03 (m, 10H), 2.61 (t, 2H), 2.21 (m, 2H), 1.93-1.68 (m, 4H).

### Example 4

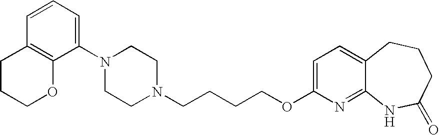

### 2-[4-(4-Chroman-8-yl-piperazin-1-yl)-butoxy]-5,6,7,9-tetrahydro-pyrido[2,3-b]azepin-8-one

2-[4-(4-Chroman-8-yl-piperazin-1-yl)-butoxy]-5,6,7,9-tetrahydro-pyrido[2,3-b]azepin-8-one was produced using a process similar to Example 2, wherein 1-chroman-8-yl-piperazine hydrochloride (US Pat. App. Pub. No. 20050043309) was substituted for 1-naphthylpiperazine in the first step of the process. The title compound crystallized in the final step as a white solid, mp. 186-87° C. ¹H-NMR (400 MHz, DMSO-d₆) δ 9.72 (s, 1H), 7.61 (d, 1H), 6.76 (m, 3H), 6.53 (d, 1H) 4.22 (t, 2H), 4.18 (t, 2H), 3.56 (m, 4H), 3.18 (m, 4H), 2.94 (m, 2H), 2.76 (t, 2H), 2.62 (t, 2H), 2.24 (m, 2H), 2.08 (m, 2H), 1.92-1.70 (m, 6H).

### Example 5

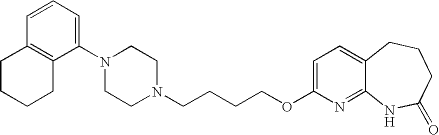

### 2-{4-[4-(5,6,7,8-Tetrahydro-naphthalen-1-yl)-piperazin-1-yl]-butoxy}-5,6,7,9-tetrahydro-pyrido[2,3-b]azepin-8-one

2-{4-[4-(5,6,7,8-Tetrahydro-naphthalen-1-yl)-piperazin-1-yl]-butoxy}-5,6,7,9-tetrahydro-pyrido[2,3-b]azepin-8-one was produced using a process similar to Example 2, wherein 1-chroman-8-yl-piperazine hydrochloride (US Pat App Pub No 20050043309) was substituted for 1-naphthylpiperazine in the first step of the process. Quantities of reagents used in the procedure were adjusted, as appropriate. The title compound crystallized in the final step as a white solid, mp. 216-217° C. ¹H-NMR (400 MHz, DMSO-d₆) δ 9.73 (s, 1H), 7.61 (d, 1H), 7.08 (t, 1H), 6.85 (t, 2H), 6.52 (d, 1H) 4.22 (t, 2H), 3.54 (t, 2H), 3.25-2.97 (m, 8H), 2.77-2.56 (m, 6H), 2.22 (m, 2H), 2.10 (m, 2H), 1.90-1.64 (m, 8H).

### Example 6

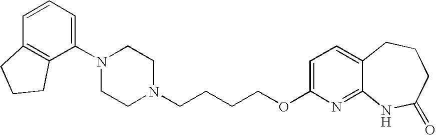

### 2-[4-(4-Indan-4-yl-piperazin-1-yl)-butoxy]-5,6,7,9-tetrahydro-pyrido[2,3-b]azepin-8-one

2-[4-(4-Indan-4-yl-piperazin-1-yl)-butoxy]-5,6,7,9-tetrahydro-pyrido[2,3-b]azepin-8-one was produced using a process similar to Example 2, wherein 1-indan-4-yl-piperazine hydrochloride (US Pat App Pub No 20050043309) was substituted for 1-naphthylpiperazine in the first step of the process. The title compound crystallized in the final step a white solid, mp. 207-208° C. ¹H-NMR (400 MHz, DMSO-d₆) δ 9.72 (s, 1H), 7.61 (d, 1H), 7.10 (t, 1H), 6.92 (d, 1H), 6.75 (d, 1H), 6.53 (d, 1H), 4.22 (t, 2H), 3.56 (m, 2H), 3.38 (m, 2H), 3.25-2.98 (m, 6H), 2.87-2.75 (m, 4H), 2.61 (t, 2H), 2.22 (m, 2H), 2.11 (m, 2H), 1.98 (m, 2H), 1.88-1.74 (m, 4H).

### Example 7

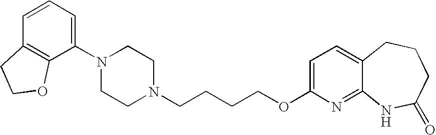

### 2-{4-[4-(2,3-Dihydro-benzofuran-7-yl)-piperazin-1-yl]-butoxy}-5,6,7,9-tetrahydro-pyrido[2,3-b]azepin-8-one

2-{4-[4-(2,3-Dihydro-benzofuran-7-yl)-piperazin-1-yl]-butoxy}-5,6,7,9-tetrahydro-pyrido[2,3-b]azepin-8-one was produced using a process similar to Example 2, wherein 1-(2,3-dihydro-benzofuran-7-yl)-piperazine hydrochloride (US Pat App Pub No. 20050043309) was substituted for 1-naphthylpiperazine in the first step of the process. The title compound crystallized in the final step as a white solid, mp. 176-177° C. ¹H-NMR (400 MHz, DMSO-d₆) δ 10.18 (s, 1H), 9.73 (s, 1H), 7.62 (d, 1H), 6.91 (d, 1H), 6.78 (t, 1H), 6.42 (d, 1H), 6.54 (d, 1H), 4.52 (t, 2H), 4.22 (t, 2H), 3.65 (m, 2H), 3.55 (m, 2H), 3.24-3.08 (m, 6H), 3.10 (m, 2H), 2.62 (t, 2H), 2.42 (m, 2H), 2.22 (m, 2H), 1.88-1.73 (m, 4H).

### Example 8

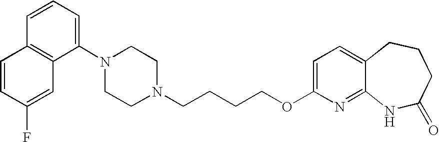

### 2-{4-[4-(7-Fluoro-naphthalen-1-yl)-piperazin-1-yl]-butoxy}-5,6,7,9-tetrahydro-pyrido[2,3-b]azepin-8-one

2-{4-[4-(7-Fluoro-naphthalen-1-yl)-piperazin-1-yl]-butoxy}-5,6,7,9-tetrahydro-pyrido[2,3-b]azepin-8-one was produced using a process similar to Example 2, wherein 1-(7-fluoro-naphthalen-1-yl)-piperazine trifluoroacetate (US Pat App Pub No. 20050043309) was substituted for 1-naphthylpiperazine in the first step of the process. The title compound crystallized in the final step as a white solid, mp. 202-203° C. ¹H-NMR (400 MHz, DMSO-d₆) δ 10.24 (s, 1H), 9.78 (s, 1H), 8.05 (q, 1H), 7.82 (m, 1H), 7.75 (d, 1H), 7.62 (d, 1H), 7.48 (m, 2H), 7.28 (d, 1H), 6.48 (d, 1H), 4.24 (t, 2H), 3.64 (m, 2H), 3.48-3.34 (m, 4H), 3.28-3.16 (m, 4H), 2.62 (t, 2H), 2.22 (m, 2H), 2.09 (m, 2H), 1.94-1.76 (m, 4H).

### Example 9

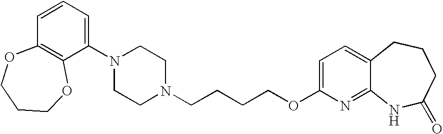

### 2-{4-[4-(3,4-Dihydro-2H-benzo[b][1,4]dioxepin-6-yl)-piperazin-1-yl]-butoxy}-5,6,7,9-tetrahydro-pyrido[2,3-b]azepin-8-one

2-{4-[4-(3,4-Dihydro-2H-benzo[b][1,4]dioxepin-6-yl)-piperazin-1-yl]-butoxy}-5,6,7,9-tetrahydro-pyrido[2,3-b]azepin-8-one was produced using a process similar to Example 2, wherein 1-(3,4-dihydro-2H-benzo[b][1,4]dioxepin-6-yl)-piperazine dihydrochloride (US Pat App Pub No. 20050043309) was substituted for 1-naphthylpiperazine in the first step of the process. The title compound crystallized in the final step as a white solid, mp. 200-201° C. ¹H-NMR (400 MHz, DMSO-d₆) δ 9.78 (s, 1H), 7.61 (d, 1H), 6.94 (t, 1H), 6.65 (m, 2H), 6.52 (d, 1H), 4.22 (t, 2H), 4.18 (m, 4H), 3.92 (s, 2H), 3.60-3.44 (m, 4H), 3.22-2.98 (m, 5H), 2.62 (t, 2H), 2.21 (m, 2H), 2.08 (m, 4H), 1.88-1.71 (m, 4H).

### Example 10

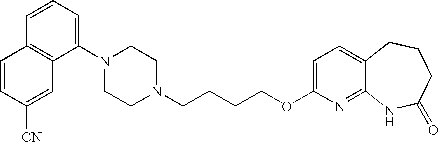

### 8-{4-[4-(8-Oxo-6,7,8,9-tetrahydro-5H-pyrido[2,3-b]azepin-2-yloxy)-butyl]-piperazin-1-yl}-naphthalene-2-carbonitrile

8-{4-[4-(8-Oxo-6,7,8,9-tetrahydro-5H-pyrido[2,3-b]azepin-2-yloxy)-butyl]-piperazin-1-yl}-naphthalene-2-carbonitrile was produced using a process similar to Example 2, wherein 8-piperazin-1-yl-naphthalene-2-carbonitrile (US Pat App Pub No. 20050043309) was substituted for 1-naphthylpiperazine in the first step of the process. The title compound crystallized in the final step as a white solid mp. 209-210° C. ¹H-NMR (400 MHz, DMSO-d₆) δ 9.72 (s, 1H), 8.63 (s, 1H), 8.13 (d, 1H), 7.81 (m, 2H), 7.69 (t, 1H), 7.62 (d, 1H), 7.38 (d, 1H), 6.55 (d, 1H), 4.23 (t, 2H), 3.63 (m, 2H), 3.54-3.38 (m, 5H), 3.31-3.18 (m, 4H), 2.61 (t, 2H), 2.22 (m, 2H), 2.10 (m, 2H), 1.96-1.76 (m, 4H).

### Example 11

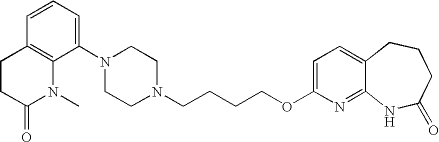

### 2-{4-[4-(1-Methyl-2-oxo-1,2,3,4-tetrahydro-quinolin-8-yl)-piperazin-1-yl]-butoxy}-5,6,7,9-tetrahydro-pyrido[2,3-b]azepin-8-one

2-{4-[4-(1-Methyl-2-oxo-1,2,3,4-tetrahydro-quinolin-8-yl)-piperazin-1-yl]-butoxy}-5,6,7,9-tetrahydro-pyrido[2,3-b]azepin-8-one was produced using a process similar to Example 2, wherein 1-(2,3-dihydro-benzofuran-7-yl)-piperazine hydrochloride (US Pat App Pub No. 20050043309) was substituted for 1-naphthylpiperazine in the first step of the process. The title compound crystallized in the final step as a white solid, mp. 184-185° C. ¹H-NMR (400 MHz, DMSO-d₆) δ 10.24 (s, 1H), 9.76 (s, 1H), 7.63 (s, 1H), 7.03 (m, 3H), 6.54 (d, 1H), 4.22 (t, 2H), 3.66 (m, 4H), 3.36-3.18 (m, 7H), 3.0 (m, 2H), 2.78 (t, 2H), 2.60 (t, 2H), 2.52 (m, 2H), 2.26 (m, 2H), 2.16 (m, 2H), 1.96-1.69 (m, 4H).

### Example 12

### N-[6-(4-Benzyloxy-butoxy)-3-formyl-pyridin-2-yl]-2,2-dimethyl-propionamide (13)

A solution of 4-Benzyloxy-butan-1-ol (12) (8.43 mL, 48 mmol) in DMF (50 mL) was treated with NaH (1.52 g, 60 mmol) at 0° C. under nitrogen. The mixture was stirred at this temperature for 15 min, and then treated with N-(6-Chloro-3-formyl-pyridin-2-yl)-2,2-dimethyl-propionamide (11), (Journal of Organic Chemistry, 55(15), 4744-50; 1990, 5.76 g, 24 mmol) in portions. After the addition was over, the mixture was left stirring for another 1 h. Aqueous NH₄Cl was added to quench the reaction. The mixture was taken up into EtOAc and washed with water, dried and concentrated. The residue was purified by column chromatography on silica gel to give the title compound (13) (6.15 g) ¹H-NMR (400 MHz, CDCl₃): 11.50 (s, 1H), 9.75 (s, 1H), 7.80 (d, 1H), 7.40-7.20 (m, 5H), 6.45 (d, 1H), 4.50 (m, 4H), 3.50 (t, 2H), 2.00-1.70 (m, 4H), 1.40 (s, 9H). Alternately, N-[6-(4-Benzyloxy-butoxy)-3-formyl-pyridin-2-yl]-2,2-dimethyl-propionamide (13) is prepared by the following method: A solution of sodium t-butoxide (3 eq., 3.41 mmol/ml DMF) was prepared, controlling the temperature within a range of 5° C. to 20° C. This solution at 5° C. was added to a solution of 4-benzyloxy-butan-1-ol (12) (1 eq., 2.39 mmole/ml DMF) over 30 minutes. After the mixture was stirred for 2 hours, a solution of N-(6-Chloro-3-formyl-pyridin-2-yl)-2,2-dimethyl-propionamide (11) (1.3 eq., 2.29 mmol/ml DMF) was added over 40 minutes, maintaining 10° C. with cooling of the mixture. After 2 hours the mixture at 20° C. was diluted with water and extracted with methyl-t-butyl ether. The organic phase was evaporated in vacuo and the residue was diluted with tetrahydrofuran and evaporated under vacuum to give a solution of crude product (13) (1 eq., assuming 1.82 mmol/ml THF), sufficiently pure to be used in the following steps.

### N-[6-(4-Benzyloxy-butoxy)-3-(2-methoxy-vinyl)-pyridin-2-yl]-2,2-dimethyl-propionamide (14)

A 1.8M solution of phenyllithium in diethyl ether (36.2 mL, 2.5 equivivalents) was added dropwise to a stirred, cooled mixture of (methoxymethyl)-triphenylphosphonium chloride (22.0 g, 65.1 mmol, 2.5 equiv) in anhydrous diethyl ether (200 mL) at −50° C. Stirring was continued for 2 h at between −50 to −30° C. and then the mixture was allowed to warm up to 0° C. over 30 minutes. N-[6-(4-Benzyloxy-butoxy)-3-formyl-pyridin-2-yl]-2,2-dimethyl-propionamide (13) (10.0 g, 26.0 mmol) dissolved in diethyl ether (50 mL) was added to the mixture, and stirring was continued for 3 h at 0° C. and then for 16 h at room temperature. Aqueous ammonium chloride solution was added to the mixture and the diethyl ether layer was separated. The aqueous solution was extracted twice with ethyl acetate. The combined organic layers were dried over Na₂SO₄ and concentrated. The residue was chromatographed on silica gel column using ethyl acetate:hexane (1:4) as eluent. The title compound (14) (E/Z mixture) was obtained as a colorless oil. ¹HNMR: δ (CDCl₃, 400 MHz): Major isomer 8.05 ((d, 1H), 7.65 (br s, 1H), 7.30-7.25 (m, 5H), 6.50 (d, 1H), 6.10 (d, 1H), 5.05 (d, 1H), 4.50 (s, 2H), 4.25 (m, 2H), 3.75 (s, 3H), 3.50 (t, 2H), 1.85-1.70 (m, 4H), 1.26 (s, 9H). Minor isomer 7.55 (d, 1H), 7.40 (br s, 1H), 7.30-7.25 (m, 5H), 6.80 (d, 1H), 6.50 (d, 1H), 5.60 (d, 1H), 4.50 (s, 2H), 4.25 (m, 2H), 3.65 (s, 3H), 3.50 (t, 2H), 1.85-1.70 (m, 4H), 1.28 (s, 9H). ESMS: 413.03, exact mass: 412.

Alternately, N-[6-(4-Benzyloxy-butoxy)-3-(2-methoxy-vinyl)-pyridin-2-yl]-2,2-dimethyl-propionamide (14) is prepared as follows: To a suspension of (methoxymethyl)-triphenylphosphonium chloride (1.5 eq., 2.4 mmol/ml THF) at −16° C. was added a 1M solution of potassium t-butoxide in THF (2.60 eq.) while temperature was maintained at near 0° C. by external cooling. After stirring for 30 minutes, a solution of crude N-[6-(4-Benzyloxy-butoxy)-3-formyl-pyridin-2-yl]-2,2-dimethyl-propionamide (13) (1 eq., 3.03 mmol/ml THF), was added over 20 minutes, while maintaining 5° C. After 1 hour, the mixture was diluted with water and extracted with methyl-t-butylether. The organic extracts were washed with brine, and evaporated to a brown oil. The oil was purified by chromatography on silica gel, eluted with a 2:1 ratio of heptane/ethyl acetate. Azeotropic evaporation from toluene gave a yellow oil, (0.75 equivalents), crude N-[6-(4-Benzyloxy-butoxy)-3-(2-methoxy-vinyl)-pyridin-2-yl]-2,2-dimethyl-propionamide (14).

### 6-(4-Benzyloxy-butoxy)-3-(2-methoxy-vinyl)-pyridin-2-ylamine (15)

N-[6-(4-Benzyloxy-butoxy)-3-(2-methoxy-vinyl)-pyridin-2-yl]-2,2-dimethyl-propionamide (14) (8.6 g), ethanol (100 mL) and 2N KOH solution (100 mL) was stirred under reflux overnight. The reaction mixture was extracted (×3) with dichloro-methane. The combined organic layer was dried over Na₂SO₄, concentrated and dried under high vacuum. The title compound (15), was obtained as a pale yellow solid which was used in the next step without further purification. ¹HNMR: δ (CDCl₃, 400 MHz): Major isomer 7.38-7.25 (m, 5H), 7.20 (d, 1H), 6.70 (d, 1H), 6.10 (d, 1H), 5.55 (d, 1H), 4.50 (s, 2H), 4.30 (br s, 1H), 4.20 (m, 2H), 3.65 (s, 3H), 3.50 (t, 2H), 1.90-1.80 (m, 4H). Minor isomer 7.60 (d, 1H), 7.38-7.20 (m, 6H), 6.10 (d, 1H), 5.05 (d, 1H), 4.50 (s, 3H), 3.70 (s, 3H), 3.50 (t, 3H), 1.90-1.80 (m, 4H). ESMS: 329.0, exact mass: 328.

Alternatively 6-(4-Benzyloxy-butoxy)-3-(2-methoxy-vinyl)-pyridin-2-ylamine (15) can be prepared by the following procedure: A solution of N-[6-(4-Benzyloxy-butoxy)-3-(2-methoxy-vinyl)-pyridin-2-yl]-2,2-dimethyl-propionamide (14) (1 eq., 0.711 mmol/ml toluene) was diluted with 95% ethanol giving a solution of 0.227 mmol/ml concentration in (14). To this was added a solution of 50% NaOH in water (10 eq) with exotherm to 55° C. The resulting dark brown mixture was heated to reflux for 1 hour, cooled to 25° C., and extracted with toluene. The combined organic extracts were washed with water, saturated brine and dried over anhydrous magnesium sulfate. Following filtration, the solvent was partially removed to give a solution of crude 6-(4-Benzyloxy-butoxy)-3-(2-methoxy-vinyl)-pyridin-2-ylamine (15) in toluene, (assumed to be 1 eq., 6.18 mmol/ml toluene).

### 2-(4-Benzyloxy-butoxy)-7,9-dihydro-1,7,9-triaza-benzocyclohepten-8-one (16)

To a stirred solution of 6-(4-Benzyloxy-butoxy)-3-(2-methoxy-vinyl)-pyridin-2-ylamine (15), (5.9 g, 18.0 mmol) in dichloromethane (80 mL) was added trichloroacetyl isocyanate (5.1 g, 27.0 mmol, 1.5 equiv) dropwise. The reaction mixture was stirred for 1 hour at room temperature and then a saturated mixture of 70% perchloric acid (20 mL) in ether (50 mL) was added. The resultant mixture was stirred for 1 hour and carefully basified with saturated NaHCO₃ solution. The organic layer was separated and the aqueous layer was extracted with DCM. The combined organic layers were dried over Na₂SO₄ and concentrated. The residue was taken in methanol (50 mL) and 1N NaOH solution (50 ML) was added and stirred for 30 min at room temperature. The reaction mixture was extracted with DCM (×3). The combined DCM layers were dried over Na₂SO₄ and concentrated. The residue was purified by flash chromatography using 30% ethyl acetate in hexane as eluent. The title compound (16), was obtained as a white solid. ¹HNMR: δ (CDCl₃, 400 MHz) 9.15 (br s, 1H), 7.85 (d, 1H), 7.75 (d, 1H), 7.35-7.20 (m, 5H), 6.65 (d, 1H), 6.45 (d, 1H), 5.70 (br s, 1H), 4.50 (s, 2H), 4.30 (t, 2H), 3.55 (t, 2H), 1.95-1.80 (m, 4H). ESMS: 339.96, exact mass: 339.

Alternately, 2-(4-Benzyloxy-butoxy)-7,9-dihydro-1,7,9-triaza-benzocyclohepten-8-one (16) is also prepared by the following method: 6-(4-Benzyloxy-butoxy)-3-(2-methoxy-vinyl)-pyridin-2-ylamine (15), (from the previous step, assumed to be 1 eq., 6.18 mmol/ml toluene) was diluted with THF to a concentration of 0.28 mmol/ml and cooled to 5° C. Trichloroacetyl isocyanate (1.2 eq.) was added neat while the temperature was maintained at 6° C. The mixture was allowed to warm to 20° C. over 1 hour following the addition after which it was cooled to 7° C. A solution of 4M HCl in methanol (5 eq) was added with external cooling to maintain less than 10° C. Following the addition, the mixture was stirred at 20° C. for 20 hours, then cooled to 10° C. and neutralized to pH 7 by addition of 1M NaOH solution. The resulting mixture was extracted with ethyl acetate, the combined extracts washed with brine, and the solvent removed under vacuum. The residue was crystallized by addition of water to a methanol solution, filtered, washed with a solution of 2:1 methanol/water. The solid was dried in a vacuum oven at 40° C. for 20 hours to give 2-(4-Benzyloxy-butoxy)-7,9-dihydro-1,7,9-triaza-benzocyclohepten-8-one (16) as a light pink-orange solid (0.735 eq.).

### 2-(4-Hydroxy-butoxy)-5,6,7,9-tetrahydro-1,7,9-triaza-benzocyclohepten-8-one (17)

2-(4-Benzyloxy-butoxy)-7,9-dihydro-1,7,9-triaza-benzocyclohepten-8-one (16), (4.1 g) was dissolved in methanol (150 mL) and 10% Pd—C (3.0 g) was added. The resultant slurry was hydrogenated at 40 psi pressure for 5 h at room temperature. The reaction mixture was filtered on a celite bed, the catalyst on the celite was washed with methanol. The combined filtrate and washings were concentrated and dried under high vacuum. The title compound (17) was obtained as a white solid which was used in the next step without further purification. ¹HNMR: δ (CD₃OD, 400 MHz) 7.45 (d, 1H), 6.30 (d, 1H), 4.25 (t, 2H), 4.00 (dd, 2H), 3.00 (t, 2H), 3.0 (t, 2H), 1.85 (m, 2H), 1.65 (m, 2H). ESMS: 252.08, exact mass: 251. Alternately, 2-(4-Hydroxy-butoxy)-5,6,7,9-tetrahydro-1,7,9-triaza-benzocyclohepten-8-one (17) is prepared by the following method: To a solution of 2-(4-Benzyloxy-butoxy)-7,9-dihydro-1,7,9-triaza-benzocyclohepten-8-one (16), (1 eq, 0.297 mmol/ml in methanol) was added Palladium on carbon (Johnson Matthey 1940 carbon, unreduced, 55% water;) in the amount 10% by weight of (16). The mixture was pressurized to 50 psig with hydrogen gas for 2.8 hours, after which the reaction was filtered to remove catalyst, washed with methanol and the solvents combined and removed under vacuum to give a semi-solid residue. To the residue was added methyl t-butyl ether and the solid was then filtered. The solid was washed then washed with methyl t-butyl ether and dried in a vacuum oven for 1 hour to give 2-(4-Hydroxy-butoxy)-5,6,7,9-tetrahydro-1,7,9-triaza-benzocyclohepten-8-one (17) as a pink solid, (0.905 eq.).

### Methanesulfonic acid 4-(8-oxo-6,7,8,9-tetrahydro-5H-1,7,9-triaza-benzocyclohepten-2-yloxy)-butyl ester (18B2)

To a suspension of 2-(4-Hydroxy-butoxy)-5,6,7,9-tetrahydro-1,7,9-triaza-benzocyclohepten-8-one (17) (1 eq., 0.1 mmol/ml in THF) was added triethyl amine (3 eq.) giving solution. Methanesulfonyl chloride (1.5 eq.) was added dropwise with an exotherm from 21° C. to 33° C. and precipitation of solids. After 10 minutes, water (5 eq.) was added with stirring over 5 minutes, followed by concentration of the mixture under vacuum to remove tetrahydrofuran. The residue was further diluted with water, stirred, filtered and washed with water. The solid was dried in a vacuum oven at 40° C. for 20 hours to give methanesulfonic acid 4-(8-oxo-6,7,8,9-tetrahydro-5H-1,7,9-triaza-benzocyclohepten-2-yloxy)-butyl ester (18B2) as a white solid (0.88 eq.).

### 2-(4-Iodo-butoxy)-5,6,7,9-tetrahydro-1,7,9-triaza-benzocyclohepten-8-one (19B2)

To a suspension of methanesulfonic acid 4-(8-oxo-6,7,8,9-tetrahydro-5H-1,7,9-triaza-benzocyclohepten-2-yloxy)-butyl ester (18B2) (1 eq., 0.155 mmol/ml in acetone) was added sodium iodide (3 eq.), followed by heating to reflux for 2 hours. The mixture was cooled to 40° C. and the solvent was partially removed under vacuum. Water was added (a slight exotherm was observed), which caused the precipitation of the desired product as a crystalline solid, After cooling to 20° C., the solid was filtered, washed with water and dried in a vacuum oven at 40° C. for 18 hours to give a white solid, 2-(4-Iodo-butoxy)-5,6,7,9-tetrahydro-1,7,9-triaza-benzocyclohepten-8-one (19B2), (0.96 eq).

### Example 13

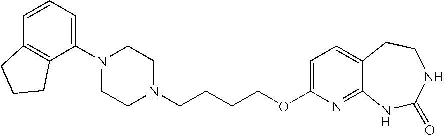

### 2-[4-(4-Indan-4-yl-piperazin-1-yl)-butoxy]-5,6,7,9-tetrahydro-1,7,9-triaza-benzocyclohepten-8-one

To 2-(4-Hydroxy-butoxy)-5,6,7,9-tetrahydro-1,7,9-triaza-benzocyclohepten-8-one (17) (0.20 g, 0.8 mmol) in dichloromethane (30 mL) and THF (5 mL) was added Dess-Martin periodinane (0.48 g, 1.12 mmol, 1.4 equiv). The mixture was stirred at room temperature for 2 h. The reaction mixture was quenched with sodium bicarbonate solution (20 mL) containing sodium thiosulfate (1.25 g, 8.0 mmol, 10.0 equiv). After extraction with dichloromethane (3×50 mL), the combined organic phases were washed with brine (20 mL), dried and concentrated to give the desired product (18), in the form of a pale yellow solid, which was dissolved in 1,2-dichloroethane (20 mL). To this solution, 1-indan-4-yl-piperazine hydrochloride (US Pat App Pub No. 20050043309, 0.23 g, 0.96 mmol, 1.2 eq), triethylamine (0.25 mL, 1.60 mmol, 2.0 equiv), NaBH(OAc)₃ (0.24 g, 1.12 mmol, 1.4 equiv) were added successively. The mixture thus obtained was stirred at room temperature for 1 h, quenched with water and sodium bicarbonate. After extraction with dichloromethane (3×50 mL), the combined organic phases were dried and concentrated. The residue was purified over silica gel column (5% MeOH in dichloromethane) to give the title compound as a colorless foam, mp: 70-72° C. ¹H-NMR δ (CDCl₃, 400 MHz): 7.35 (d, 1H), 7.10 (t, 1H), 6.90 (d, 1H), 6.75 (d, 1H), 6.25 (d, 1H), 4.25 (t, 2H), 4.06 (t, 2H), 3.10-2.90 (m, 6H), 2.90-2.80 (m, 4H), 2.60 (br s, 4H), 2.45 (t, 2H), 2.05 (m, 2H0, 1.80-1.60 (m, 4H). HPLC: 92.93%. MS: 436.09, exact mass: 435.

### Example 14

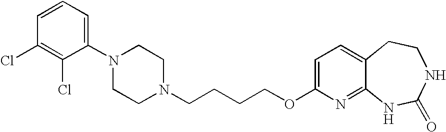

### 2-{4-[4-(2,3-Dichloro-phenyl)-piperazin-1-yl]-butoxy}-5,6,7,9-tetrahydro-1,7,9-triaza-benzocyclohepten-8-one

2-{4-[4-(2,3-Dichloro-phenyl)-piperazin-1-yl]-butoxy}-5,6,7,9-tetrahydro-1,7,9-triaza-benzocyclohepten-8-one was produced using a process similar to Example 13, wherein 1-(2,3-Dichloro-phenyl)-piperazine hydrochloride, (Lancaster) was added in place of 1-indan-4-yl-piperazine hydrochloride, in the same step of the process. The residue purified on the silica gel column in the final step of the process was the title compound in the form of a colorless oil, which was converted to HCl salt. ¹H-NMR (400 MHz, DMSO-d₆): 10.40 (s, 1H), 8.10 (s, 1H), 7.40 (m, 4H), 7.20 (m, 1H), 6.30 (d, 1H), 4.23 (t, 2H), 3.60 (m, 2H), 3.40 (m, 2H), 3.20 (m, 8H), 2.80 (m, 2H), 1.90-1.70 (m, 4H). HPLC: 93.99%. m.p.: 197-199° C. MS: 464.

### Example 15

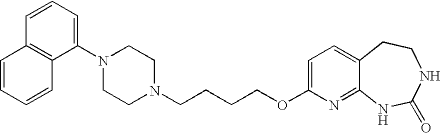

### 2-[4-(4-Naphthalen-1-yl-piperazin-1-yl)-butoxy]-5,6,7,9-tetrahydro-1,7,9-triaza-benzocyclohepten-8-one

2-[4-(4-Naphthalen-1-yl-piperazin-1-yl)-butoxy]-5,6,7,9-tetrahydro-1,7,9-triaza-benzocyclohepten-8-one was produced using a process similar to Example 13, wherein 1-naphthylpiperazine hydrochloride was added in place of 1-indan-4-yl-piperazine hydrochloride, in the same step of the process. The residue from the dichloromethane extraction step purified on the silica gel column in the final step of the process was the title compound in the form of a colorless oil, which was converted to HCl salt. ¹H-NMR (400 MHz, DMSO-d₆): 10.20 (s, 1H), 8.15 (m, 2H), 7.90 (d, 1H), 7.65 (d, 1H), 7.54 (m, 2H), 7.45 (m, 1H), 7.42 (d, 1H), 7.35 (s, 1H), 7.20 (d, 1H), 6.30 (d, 1H), 4.23 (t, 2H), 3.40 (m, 2H), 3.50-3.10 (m, 10H, 2.80 (m, 2H), 1.90-1.70 (m, 4H). MS: 446. m.p.: 188-190° C.

### Example 16

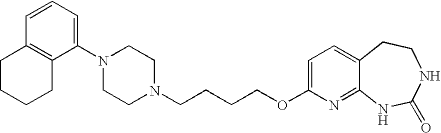

### 2-{4-[4-(5,6,7,8-Tetrahydro-naphthalen-1-yl)-piperazin-1-yl]-butoxy}-5,6,7,9-tetrahydro-1,7,9-triaza-benzocyclohepten-8-one

2-{4-[4-(5,6,7,8-Tetrahydro-naphthalen-1-yl)-piperazin-1-yl]-butoxy}-5,6,7,9-tetrahydro-1,7,9-triaza-benzocyclohepten-8-one was produced using a process similar to Example 13, wherein 1-(5,6,7,8-tetrahydro-naphthalen-1-yl)-piperazine hydrochloride (US Pat App Pub No. 20050043309) was added in place of 1-indan-4-yl-piperazine hydrochloride, in the same step of the process. The residue from the dichloromethane extraction step was purified over a silica gel column (5% MeOH in dichloromethane) to give pale yellow oil which was further purified over a second silica gel column (dichloromethane:methanol:TH:Et₃N, 8:1:2:0.2) to give the title compound as a colorless foam which was converted to the HCl salt. mp: 185-186° C. ¹H-NMR (400 MHz, CDCl₃): 7.50-7.40 (br s, 1H), 7.25 (m, 2H), 7.05 (t, 1H), 7.00 (d, 1H), 6.90 (d, 1H), 6.35 (d, 1H), 6.00-5.90 (br s, 1H), 4.25 (t, 2H), 3.65-3.60 (m, 4H), 3.40 (m, 2H), 3.20-3.10 (m, 4H), 2.90 (m, 2H), 2.80-2.60 (m, 4H), 2.30-2.20 (m, 4H), 1.90 (m, 2H), 1.90-1.70 (m, 4H). HPLC: 91.81%. MS: 450.13 (M+H)⁺

### Example 17

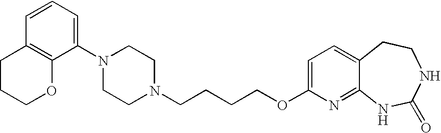

### 2-[4-(4-Chroman-8-yl-piperazin-1-yl)-butoxy]-5,6,7,9-tetrahydro-1,7,9-triaza-benzocyclohepten-8-one

2-[4-(4-Chroman-8-yl-piperazin-1-yl)-butoxy]-5,6,7,9-tetrahydro-1,7,9-triaza-benzocyclohepten-8-one was produced using a process similar to Example 13, wherein 1-chroman-8-yl-piperazine hydrochloride (US Pat App Pub No. 20050043309) was added in place of 1-indan-4-yl-piperazine hydrochloride, in the same step of the process. The residue from the dichloromethane extraction step was purified over a silica gel column (5% MeOH in dichloromethane) to give a pale yellow oil which was further purified over a second silica gel column (dichloromethane:methanol:TH:Et₃N, 8:1:2:0.2) to give the title compound as a colorless foam which was converted to the HCl salt, mp: 154-156° C. ¹H-NMR (400 MHz, CDCl₃): 7.30 (m, 1H), 7.05 (br s, 1H), 6.80-6.70 (m, 2H), 6.35 (d, 1H), 5.60 (br s, 1H), 4.25 (m, 4H), 3.45 (m, 2H), 3.10 (m, 4H), 2.90 (m, 2H), 2.80 (t, 2H), 2.70 (m, 4H), 2.50 (m, 2H), 2.00 (m, 2H), 1.80-1.60 (m, 4H). HPLC: 93.43%. MS: 452.08 (M+H)⁺.

### Example 18

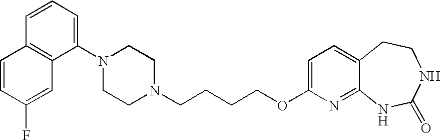

### 2-{4-[4-(7-Fluoro-naphthalen-1-yl)-piperazin-1-yl]-butoxy}-5,6,7,9-tetrahydro-1,7,9-triaza-benzocyclohepten-8-one

2-{4-[4-(7-Fluoro-naphthalen-1-yl)-piperazin-1-yl]-butoxy}-5,6,7,9-tetrahydro-1,7,9-triaza-benzocyclohepten-8-one was produced using a process similar to Example 13, wherein 1-(7-fluoro-naphthalen-1-yl)-piperazine hydrochloride (US Pat App Pub No 20050043309) was added in place of 1-indan-4-yl-piperazine hydrochloride, in the same step of the process. The residue from the dichloromethane extraction step was purified over a silica gel column in the final step of the process was the title compound in the form of a colorless foam which was converted to its HCl salt by adding 1.0 M ethereal HCl solution, mp: 234° C. ¹H-NMR δ (CDCl₃, 400 MHz): 8.50 (br s, 1H), 7.80 (m, 2H), 7.55 (d, 1H), 7.35 (m, 2H), 7.21 (m, 1H), 7.10 (d, 1H), 6.25 (d, 1H), 5.60 (br s, 1H), 4.25 (t, 2H), 4.05 (t, 2H), 3.20 (br s, 4H), 3.00 (t, 2H), 2.80 (br s, 4H), 2.50 (t, 2H), 1.90-1.65 (m, 4H). HPLC: 90.72%. MS: 464.18, exact mass: 463.

Alternately, 2-{4-[4-(7-Fluoro-naphthalen-1-yl)-piperazin-1-yl]-butoxy}-5,6,7,9-tetrahydro-1,7,9-triaza-benzocyclohepten-8-one is prepared as follows: A combined suspension in acetonitrile of 1-(7-fluoro-naphthalen-1-yl)-piperazine hydrochloride (US Pat App Pub No 20050043309, 1.05 eq., 0.24 mmol/ml), 2-(4-Iodo-butoxy)-5,6,7,9-tetrahydro-1,7,9-triaza-benzocyclohepten-8-one (19B2) (1.0 eq., 0.23 mmol/ml) and potassium carbonate (3.0 eq., 0.68 mmol/ml) was heated to 70° C. for for 4 hours. The mixture was allowed to cool during the addition of waterwhich gave a thick suspension. The mixture was further cooled to 20° C., filtered, washed with a 1:1 mixture of water/acetonitrile and dried in a vacuum oven at 50° C. for 18 hours to give a white solid, (0.95 eq.). Further improvement in purity was obtained by recrystallization from chloroform at elevated temperature.

### Example 19

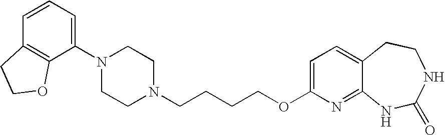

### 2-{4-[4-(2,3-Dihydro-benzofuran-7-yl)-piperazin-1-yl]-butoxy}-5,6,7,9-tetrahydro-1,7,9-triaza-benzocyclohepten-8-one

2-{4-[4-(2,3-Dihydro-benzofuran-7-yl)-piperazin-1-yl]-butoxy}-5,6,7,9-tetrahydro-1,7,9-triaza-benzocyclohepten-8-one was produced using a process similar to Example 13, wherein 1-(2,3-dihydro-benzofuran-7-yl)-piperazine hydrochloride (US Pat App Pub No. 20050043309) was added in place of 1-indan-4-yl-piperazine hydrochloride, in the same step of the process. The residue from the dichloromethane extraction was purified over a silica gel column (5% MeOH in dichloromethane) to give a pale yellow oil which was further purified over silica gel column (ethylacetate:dichloromethane:methanol, 2:2:1) to give the title compound as a colorless foam, mp: 72-73° C. ¹H-NMR δ (CDCl₃, 400 MHz): 8.45 (br s, 1H), 7.40 (d, 1H), 6.90-6.60 (m, 3H), 6.25 (d, 1H), 5.15 (br s, 1H), 4.60 (t, 2H), 4.30-4.05 (m, 4H), 3.40-3.00 (m, 8H), 2.70 (br s, 4H), 2.45 (t, 2H), 1.90-1.60 (m, 4H). HPLC: 90.61%. MS: 438.1, exact mass: 437. Elemental Analysis Cacld for C₂₄H₃₁N₅O₃: C, 65.88; H, 7.14; N, 16.01. Found: C, 65.51; H, 7.01; N, 15.45.

### Example 20

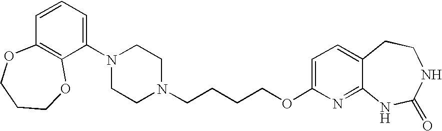

### 2-{4-[4-(3,4-Dihydro-2H-benzo[b][1,4]dioxepin-6-yl)-piperazin-1-yl]-butoxy}-5,6,7,9-tetrahydro-1,7,9-triaza-benzocyclohepten-8-one

2-{4-[4-(3,4-Dihydro-2H-benzo[b][1,4]dioxepin-6-yl)-piperazin-1-yl]-butoxy}-5,6,7,9-tetrahydro-1,7,9-triaza-benzocyclohepten-8-one was produced using a process similar to Example 13, wherein 1-(3,4-dihydro-2H-benzo[b][1,4]dioxepin-6-yl)-piperazine hydrochloride (US Pat App Pub No 20050043309) was added in place of 1-indan-4-yl-piperazine hydrochloride, in the same step of the process. The residue from the dichloromethane extraction step was purified over a silica gel column (5% MeOH in dichloromethane) to give the title compound as a colorless foam, mp: 78-79° C. ¹H-NMR δ (CDCl₃, 400 MHz): 8.50 (br s, 1H), 7.40 (d, 1H), 6.90 (t, 1H), 6.65 (m, 2H), 6.25 (d, 1H), 5.10 (br s, 1H), 4.30 (m, 6H), 4.05 (t, 2H), 3.10 (br s, 4H), 3.00 (t, 2H), 2.70 (br s, 4H), 2.50 (t, 2H), 2.10 (t, 2H), 1.80-1.65 (m, 4H). HPLC: 90.24%. ESMS: 468.04, exact mass: 467. Elemental Analysis Cacld. for C₂₅H₃₃N₅O₄.0.5H₂O: C, 63.01; H, 7.19; N, 14.69. Found: C, 62.85; H, 7.22; N, 14.60.

### Example 21

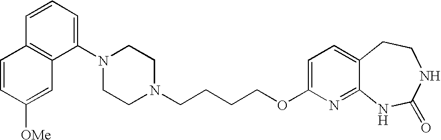

### 2-{4-[4-(7-Methoxy-naphthalen-1-yl)-piperazin-1-yl]-butoxy}-5,6,7,9-tetrahydro-1,7,9-triaza-benzocyclohepten-8-one

2-{4-[4-(7-Methoxy-naphthalen-1-yl)-piperazin-1-yl]-butoxy}-5,6,7,9-tetrahydro-1,7,9-triaza-benzocyclohepten-8-one was produced using a process similar to Example 13, wherein 1-(7-methoxy-naphthalen-1-yl)-piperazine hydrochloride (US Pat App Pub No 20050043309) was added in place of 1-indan-4-yl-piperazine hydrochloride, in the same step of the process. The residue from the dichloromethane extraction step was purified over a silica gel column (5% MeOH in dichloromethane) to give the title compound as a colorless sticky solid which was converted into its HCl salt by adding 1.0 M ethereal HCl solution, mp: 155-158° C. ¹H-NMR δ (CDCl₃, 400 MHz): 8.50 (br s, 1H), 7.75 (d, 1H), 7.50 (m, 2H), 7.35 (d, 1H), 7.25 (m, 1H), 7.10 (m, 2H), 6.25 (d, 1H), 5.15 (br s, 1H), 4.25 (t, 2H), 4.10 (t, 2H), 3.90 (s, 3H), 3.10 (br s, 4H), 3.00 (t, 2H), 2.80 (br s, 4H), 2.55 (t, 2H), 1.90-1.70 (m, 4H). HPLC: 90.58%. ESMS: 476.28, exact mass: 475.

### Example 22

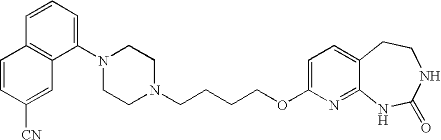

### 8-{4-[4-(8-Oxo-6,7,8,9-tetrahydro-5H-1,7,9-triaza-benzocyclohepten-2-yloxy)-butyl]-piperazin-1-yl}-naphthalene-2-carbonitrile

8-{4-[4-(8-Oxo-6,7,8,9-tetrahydro-5H-1,7,9-triaza-benzocyclohepten-2-yloxy)-butyl]-piperazin-1-yl}-naphthalene-2-carbonitrile was produced using a process similar to Example 13, wherein 8-piperazin-1-yl-naphthalene-2-carbonitrile hydrochloride (US Pat App Pub No 20050043309) was added in place of 1-indan-4-yl-piperazine hydrochloride, in the same step of the process. The residue from the dichloromethane extraction step was purified over a silica gel column (5% MeOH in dichloromethane) to give the title compound as a colorless sticky solid which was converted into its HCl salt by adding 1.0 M ethereal HCl solution, mp: 168-170° C. ¹H-NMR δ (CDCl₃, 400 MHz): 8.60 (s, 1H), 8.50 (br s, 1H), 7.90 (d, 1H), 7.60 (m, 3H), 7.40 (d, 1H), 7.20 (m, 1H), 6.25 (d, 1H), 5.15 (br s, 1H), 4.25 (t, 2H), 4.10 (t, 2H), 3.10 (br s, 4H), 3.00 (t, 2H), 2.80 (br s, 4H), 2.55 (t, 2H), 1.90-1.70 (m, 4H). HPLC: 95.26%. ESMS: 471.27, exact mass: 470.

### Example 23

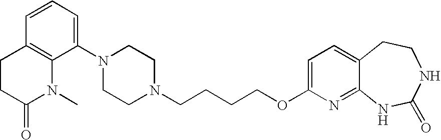

### 2-{4-[4-(1-Methyl-2-oxo-1,2,3,4-tetrahydro-quinolin-8-yl)-piperazin-1-yl]-butoxy}-5,6,7,9-tetrahydro-1,7,9-triaza-benzocyclohepten-8-one

2-{4-[4-(1-Methyl-2-oxo-1,2,3,4-tetrahydro-quinolin-8-yl)-piperazin-1-yl]-butoxy}-5,6,7,9-tetrahydro-1,7,9-triaza-benzocyclohepten-8-one was produced using a process similar to Example 13, wherein 1-methyl-8-piperazin-1-yl-3,4-dihydro-1H-quinolin-2-one hydrochloride (US Pat App Pub No 20050043309) was added in place of 1-indan-4-yl-piperazine hydrochloride, in the same step of the process. The residue from the dichloromethane extraction step was purified over a silica gel column (7% MeOH in dichloromethane and repurified with 4% MeOH in dichloromethane) to give the title compound as a colorless foam, mp: 80-82° C. ¹H-NMR δ (CDCl₃, 400 MHz): 7.40 (d, 1H), 7.10 (d, 2H), 7.00 (m, 1H), 6.25 (d, 1H), 4.25 (t, 2H), 4.10 (t, 2H), 3.80 (br s, 4H), 3.40 (s, 3H), 3.30-2.90 (m, 8H), 2.80 (m, 2H), 2.55 (t, 2H), 1.90-1.70 (m, 4H). HPLC: 90.77%. ESMS: 479.25, exact mass: 478.

### Example 24

### 4-Methoxy-2-(nitrophenyl)acetonitrile (20)

To a stirred solution of 1-Bromomethyl-4-methoxy-2-nitro-benzene (19) (Journal of Organic Chemistry, 49(7), 1238-46; 1984, 6.50 g, 26.4 mmol) in tetrahydrofuran (80 mL) and ethanol (20 mL) at 0° C. was added a solution of potassium cyanide (3.44 g, 52.8 mmol) in water (20 mL). The reaction mixture was stirred at 0° C. for 1 h and a further 3 h at room temperature. The reaction mixture was diluted with water (300 mL) and the aqueous phase was extracted with dichloromethane (3×200 mL). The combined organic extracts were washed with brine, dried over sodium sulfate and filtered. Solvent removal in vacuo followed by purification of the residue by silica gel chromatography (eluant: 90:10 hexanes/ethyl acetate) afforded the title compound (20) as a white solid: ¹H NMR (CDCl₃) δ 7.70 (d, J=2.7 Hz, 1H), 7.60 (d, J=8.6 Hz, 1H), 7.23 (dd, J=8.6, 2.7 Hz, 1H), 4.12 (s, 2H), 3.90 (s, 3H); MS (ESI) m/z 193 [C₉H₈N₂O₃+H]⁺.

### 2-(4-Methoxy-2-nitro-phenyl)-ethylamine (21A)

To a stirred solution of the (4-Methoxy-2-nitro-phenyl)-acetonitrile (20) (3.90 g, 20.3 mmol) in dry tetrahydrofuran (75 mL) was added borane-tetrahydrofuran complex (41 mL, 41 mmol, 1.0 M solution in tetrahydrofuran). The reaction mixture was heated to reflux for 4 h and, after cooling to room temperature, quenched by the addition of methanol (10 mL), followed by a 2 M hydrochloric acid solution (40 mL). The reaction mixture was heated to reflux for 1 h, cooled to room temperature and was made alkaline by the addition of aqueous 1 M sodium hydroxide. The aqueous layer was extracted with dichloromethane (3×100 mL) and the combined organics were dried over sodium sulfate and filtered. Solvent removal in vacuo afforded crude title compound (21A) as a colorless foam: ¹H NMR (CDCl₃) δ 7.40-7.36 (m, 1H), 7.31-7.26 (m, 1H), 7.11-7.08 (m, 1H), 3.85 (s, 3H), 2.96 (m, 4H); ¹³C NMR (CDCl₃) δ 158.2, 149.7, 133.0, 126.5, 119.6, 109.2, 55.6, 42.8, 36.4; MS (ESI) m/z 197 [C₉H₁₂N₂O₃+H]⁺. (CDCl₃) δ 159.2, 145.9, 131.0, 117.0, 103.9, 101.6, 55.1, 41.8, 34.4; MS (ESI) m/z 167 [C₉H₁₄N₂O+H]⁺.

### [2-(4-Methoxy-2-nitrophenyl)ethyl]carbamic Acid tert-Butyl Ester (22)

To a stirred solution of 2-(4-Methoxy-2-nitro-phenyl)-ethylamine (21A) (2.25 g, 11.5 mmol) in dry tetrahydrofuran (28 mL) was added a solution of di-tert-butyldicarbonate (3.00 g, 13.8 mmol) in tetrahydrofuran (4 mL). The reaction mixture was stirred at room temperature for 16 h and was diluted with water (100 mL) and 1 M hydrochloric acid solution (50 mL). The aqueous layer was extracted with ethyl acetate (3×100 mL) and the combined organic extracts were washed with brine (100 mL), dried over sodium sulfate and filtered. Solvent removal in vacuo followed by purification of the residue by silica gel chromatography (eluant: 80:20 hexanes/ethyl acetate) afforded the title compound (22) as a colorless oil: ¹H NMR (CDCl₃) δ 7.44 (d, J=2.5 Hz, 1H), 7.29 (d, J=8.5 Hz, 1H), 7.09 (dd, J=8.5, 2.7 Hz, 1H), 4.90-4.89 (m, 1H), 3.85 (s, 3H), 3.41 (q, J=6.7 Hz, 2H), 3.02 (t, J=7.1 Hz, 2H), 1.42 (s, 9H); MS (ESI) m/z 297 [C₁₄H₂₀N₂O₅+H]⁺.

### [2-(4-Methoxy-2-nitrophenyl)ethyl]methyl-carbamic Acid tert-Butyl Ester (23)

To a stirred suspension of sodium hydride (0.36 g, 60% in mineral oil, 9.0 mmol) in dry tetrahydrofuran (10 mL) was added a solution of [2-(4-Methoxy-2-nitrophenyl)ethyl]carbamic Acid tert-Butyl Ester (22) (1.90 g, 6.42 mmol) in tetrahydrofuran (10 mL) and iodomethane (1.37 g, 9.63 mmol). The reaction mixture was stirred at room temperature for 16 h and quenched with saturated ammonium chloride solution (100 mL). The layers were separated and the aqueous layer was extracted with ethyl acetate (3×50 mL). The combined organic extracts were washed with brine (250 mL), dried over sodium sulfate, filtered and concentrated in vacuo to provide the title compound (23) (product contained residual mineral oil) as a pale yellow oil: ¹H NMR (CDCl₃) δ 7.48-7.46 (m, 1H), 7.40-7.20 (m, 1H), 7.10-7.06 (m, 1H), 3.85 (s, 3H), 3.49 (t, J=6.8 Hz, 2H), 3.02 (t, J=6.5 Hz, 2H), 2.86 (s, 3H), 1.36 (s, 9H); MS (ESI) m/z 311 [C₁₅H₂₂N₂O₅+H]⁺.

### 5-Methoxy-2-(2-methylaminoethyl)phenylamine Hydrochloride (24)

To a stirred solution of [2-(4-Methoxy-2-nitrophenyl)ethyl]methyl-carbamic Acid tert-Butyl Ester (23) (2.10 g, 6.77 mmol) in 1,4-dioxane (10 mL) was added 4 M hydrogen chloride in 1,4-dioxane (40 mL). The reaction mixture was stirred at 80° C. for 1 h and cooled to room temperature. The solvent was removed in vacuo and the resulting residue was triturated with ether. The white solid was collected by filtration and dried in a vacuum oven at 40° C. overnight to give the amine (1.4 g, 84%): MS (ESI) m/z 211 [C₁₀H₁₄N₂O₃+H]⁺.

To a Parr bottle containing wet 10% palladium on carbon (0.14 g) was added methanol (20 mL) under an atmosphere of nitrogen. The mixture was shaken with hydrogen (40 psi) for 5 min to pre-reduce the catalyst. A solution of the above amine (1.40 g, 5.68 mmol) in methanol (100 mL) was added to the pre-reduced catalyst and the reaction mixture was shaken for 2 h under hydrogen (50 psi). The mixture was filtered through a pad of diatomaceous earth and the filtrate was concentrated in vacuo to give the title compound (24) as a pale yellow solid: ¹H NMR (CD₃OD) δ 6.88 (d, J=8.3 Hz, 1H), 6.34 (d, J=2.5 Hz, 1H), 6.17 (dd, J=8.3, 2.5 Hz, 1H), 3.62 (s, 3H), 3.09-3.04 (m, 2H), 2.89-2.84 (m, 2H), 2.63 (s, 3H); MS (ESI) m/z 181 [C₁₀H₁₆N₂O+H]⁺.

### 8-Methoxy-3-methyl-1,3,4,5-tetrahydrobenzo[d][1,3]diazepin-2-one (25)

5-Methoxy-2-(2-methylaminoethyl)phenylamine Hydrochloride (24) (1.3 g, 7.2 mmol), triethylamine (2.00 mL, 14.4 mmol), 1,1′-carbonyldiimidazole (1.80 g, 10.8 mmol) and tetrahydrofuran (40 mL) were placed in a 10 mL glass vessel, sealed and the mixture was stirred. The sample was subjected to a sequential process of microwave irradiation (using CEM Explorer Microwave Technology) for 20 min, maintaining a temperature of 150° C. After cooling to room temperature, the reaction mixture was diluted with ethyl acetate (100 mL) and 1 M hydrochloric acid (100 mL). The organic phase was separated and the aqueous layer was extracted with ethyl acetate (3×100 mL). The combined organic extracts were washed with brine, dried over sodium sulfate and filtered. Solvent removal in vacuo followed by purification of the residue by silica gel chromatography (eluant: 95:5 ethyl acetate/methanol) afforded the title compound (25) as a pale yellow solid: ¹H NMR (CDCl₃) δ 8.60 (s, 1H), 6.89 (d, J=8.4 Hz, 1H), 6.62 (d, J=2.4 Hz, 1H), 6.44 (dd, J=8.4, 2.5 Hz, 1H), 3.75 (s, 3H), 3.47-3.44 (m, 2H), 3.05 (s, 3H), 2.95-2.92 (m, 2H); MS (ESI) m/z 207 [C₁₁H₁₄N₂O₂+H]⁺.

### 8-Hydroxy-3-methyl-1,3,4,5-tetrahydrobenzo[d][1,3]diazepin-2-one (26)

To a stirred solution of 8-Methoxy-3-methyl-1,3,4,5-tetrahydrobenzo[d][1,3]diazepin-2-one (25) (0.45 g, 2.2 mmol) in dichloromethane (20 mL) at −78° C. was added dropwise boron tribromide (5.0 mL, 5.0 mmol, 1.0 M solution in dichloromethane). The reaction mixture was allowed to warm to room temperature overnight. After stirring for 16 h, the reaction was quenched by the addition of ether. The mixture was then poured onto ice, stirred for 30 min and the organic layer was separated. The aqueous layer was extracted with ethyl acetate (5×50 mL) and the combined organic extracts were dried over sodium sulfate, filtered and concentrated in vacuo to afford the title compound (26) the title compound as a pale yellow solid: ¹H NMR (CD₃OD) δ 6.87 (d, J=8.4 Hz, 1H), 6.39-6.35 (m, 2H), 3.50-3.47 (m, 2H), 2.99 (s, 3H), 2.95-2.92 (m, 2H); MS (ESI) m/z 193 [C₁₀H₁₂N₂O₂+H]⁺.

### 8-(4-Chlorobutoxy)-3-methyl-1,3,4,5-tetrahydro-benzo[d][1,3]diazepin-2-one (27)

To a stirred solution of 8-Hydroxy-3-methyl-1,3,4,5-tetrahydrobenzo[d][1,3]diazepin-2-one (26) (0.40 g, 2.1 mmol) in ethanol (30 mL) was added cesium carbonate (1.4 g, 4.2 mmol) and 1-bromo-4-chlorobutane (0.75 mL, 6.2 mmol). The reaction mixture was heated to reflux for 16 h and then diluted with water (100 mL). The mixture was stirred for 1 h and the precipitate was collected by filtration. The white solid was dried in a vacuum oven at 45° C. overnight to give the title compound (27) ¹H NMR (CDCl₃) δ 6.94 (d, J=8.3 Hz, 1H), 6.78 (s, 1H), 6.47 (dd, J=8.3, 2.2 Hz, 1H), 6.31 (d, J=2.0 Hz, 1H), 3.95 (t, J=5.7 Hz, 2H), 3.61 (t, J=6.0 Hz, 2H), 3.49-3.47 (m, 2H), 3.04 (s, 3H), 2.99-2.96 (m, 2H), 1.99-1.90 (m, 4H); MS (ESI) z/z 283 [C₁₄H₁₉ClN₂O₂+H]⁺.

### Example 25

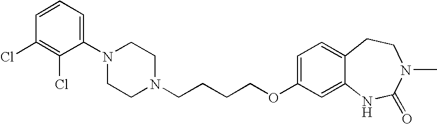

### 8-{4-[4-(2,3-Dichlorophenyl)piperazin-1-yl]butoxy}-3-methyl-1,3,4,5-tetrahydro-benzo[d][1,3]diazepin-2-one

To a stirred solution of chloride (27) (0.50 g, 1.8 mmol) in acetonitrile (60 mL) was added dichlorophenyl piperazine hydrochloride (0.56 g, 2.1 mmol), sodium iodide (0.53 g, 3.5 mmol) and potassium carbonate (0.73 g, 5.3 mmol). The reaction mixture was heated to reflux for 48 h, cooled to room temperature and diluted with water (140 mL). The mixture was stirred for 4 h and the precipitate was collected by filtration. The white solid was dried in a vacuum oven at 45° C. overnight to give the title compound, mp 139-140° C. (recrystallized from acetonitrile); ¹H NMR (DMSO-d₆) δ 8.45 (s, 1H), 7.31-7.28 (m, 2H), 7.17-7.10 (m, 1H), 6.94 (d, J=8.4 Hz, 1H), 6.64 (d, J=2.3 Hz, 1H), 6.43 (dd, J=8.3, 2.4 Hz, 1H), 3.90 (t, J=6.2 Hz, 2H), 3.40-3.38 (m, 2H), 2.98-2.97 (m, 4H), 2.88 (s, 3H), 2.86-2.85 (m, 2H), 2.52-2.50 (m, 4H), 2.38 (t, J=7.0 Hz, 2H), 1.76-1.67 (m, 2H), 1.62-1.55 (m, 2H); MS (ESI) m/z 477 [C₂₄H₃₀Cl₂N₄O₂+H]⁺

### Example 26

### 2-(2-Aminoethyl)-5-methoxyphenylamine (21B)

To a stirred solution of the nitrile 20 (3.90 g, 20.3 mmol) in dry tetrahydrofuran (75 mL) was added borane-tetrahydrofuran complex (41 mL, 41 mmol, 1.0 M solution in tetrahydrofuran). The reaction mixture was heated to reflux for 4 h and, after cooling to room temperature, quenched by the addition of methanol (10 mL), followed by a 2 M hydrochloric acid solution (40 mL). The reaction mixture was heated to reflux for 1 h, cooled to room temperature and was made alkaline by the addition of aqueous 1 M sodium hydroxide. The aqueous layer was extracted with dichloromethane (3×100 mL) and the combined organics were dried over sodium sulfate and filtered. Solvent removal in vacuo afforded crude 2-(4-methoxy-2-nitrophenyl)ethylamine as a colorless foam: ¹H NMR (CDCl₃) δ 7.40-7.36 (m, 1H), 7.31-7.26 (m, 1H), 7.11-7.08 (m, 1H), 3.85 (s, 3H), 2.96 (m, 4H); ¹³C NMR (CDCl₃) δ 158.2, 149.7, 133.0, 126.5, 119.6, 109.2, 55.6, 42.8, 36.4; MS (EST) m/z 197 [C₉H₁₂N₂O₃+H]⁺.

To a Parr bottle containing wet 10% palladium on carbon (0.9 g) was added methanol (80 mL) under an atmosphere of nitrogen. The mixture was shaken with hydrogen (40 psi) for 10 min to pre-reduce the catalyst. A solution of the above amine (4.20 g, 21.4 mmol) in methanol (100 mL) was added to the pre-reduced catalyst and the reaction mixture was shaken for 1 h under hydrogen (40 psi) atmosphere. The mixture was filtered through a pad of diatomaceous earth and concentrated to afford the aniline 22B as a pale yellow oil: ¹H NMR (CDCl₃) δ 6.89 (d, J=8.2 Hz, 1H), 6.29-6.23 (m, 2H), 3.73 (s, 3H), 2.94 (t, J=6.9 Hz, 2H), 2.62 (t, J=6.7 Hz, 2H); ¹³C NMR (CDCl₃) δ 159.2, 145.9, 131.0, 117.0, 103.9, 101.6, 55.1, 41.8, 34.4; MS (ESI) m/z 167 [C₉H₁₄N₂O+H]⁺.

### 8-Methoxy-1,3,4,5-tetrahydrobenzo[d][1,3]diazepin-2-one (28)

To a stirred solution of 2-(2-Aminoethyl)-5-methoxyphenylamine (21B) (4.30 g, 25.9 mmol) in dry tetrahydrofuran (100 mL) was added solid 1,1′-carbonyldiimidazole (5.00 g, 31.1 mmol) in small portions over 5 min. The reaction mixture was heated to reflux for 20 h, cooled to room temperature and was diluted with ethyl acetate (200 mL) and a 1 M hydrochloric acid solution (100 mL). The organic phase was separated and the aqueous layer was extracted with ethyl acetate (3×100 mL). The combined organic extracts were washed with brine, dried over sodium sulfate and filtered. Solvent removal in vacuo followed by purification of the residue by silica gel chromatography (eluant: 95:5 dichloromethane/methanol) afforded the title compound, as a pale yellow solid: ¹H NMR (CDCl₃) δ 9.54 (br s, 1H), 7.00 (d, J=8.4 Hz, 1H), 6.79 (d, J=2.4 Hz, 1H), 6.64 (dd, J=8.4, 2.4 Hz, 1H), 6.43 (br s, 1H), 3.78 (s, 3H), 3.60-3.59 (m, 2H), 3.07-3.04 (m, 2H); MS (ESI) m/z 193 [C₁₀H₁₂N₂O₂+H]⁺.

### 8-Hydroxy-1,3,4,5-tetrahydrobenzo[d][1,3]diazepin-2-one (29)

To a stirred solution of 8-Methoxy-1,3,4,5tetrahydrobenzo[d][1,3]-diazepin-2-one (28). (1.75 g, 9.10 mmol) in dichloromethane (300 mL) cooled to −78° C. was added dropwise boron tribromide (20 mL, 1.0 M solution in dichloromethane). The reaction was allowed to warm to room temperature overnight. After stirring for 16 h, the reaction was quenched by the addition of ether. The mixture was then poured onto ice, stirred for 30 min and the organic layer was separated. The aqueous layer was extracted with ethyl acetate (5×50 mL) and the combined organic extracts were dried over sodium sulfate, filtered and concentrated in vacuo to afford the title compound, as a pale yellow solid: ¹H NMR (DMSO-d₆) δ 9.14 (br s, 1H), 8.47 (s, 1H), 6.92 (br s, 1H), 6.78 (d, J=8.2 Hz, 1H), 6.44 (d, J=2.4 Hz, 1H), 6.24 (dd, J=8.2, 2.4 Hz, 1H), 3.18-3.13 (m, 2H), 2.77 (t, J=4.8 Hz, 2H); MS (ESI) m/z 179 [C₉H₁₀N₂O₂+H]⁺.

### General procedure for alkylation of 8-Hydroxy-1,3,4,5-tetrahydrobenzo[d][1,3]-diazepin-2-one (29) with a dihaloalkane

A general alkylation procedure was used to alkylate 8-hydroxy-1,3,4,5-tetrahydrobenzo[d][1,3]diazepin-2-one (29) with a dihaloalkane, as follows. To a solution of 8-hydroxy-1,3,4,5-tetrahydrobenzo[d][1,3]diazepin-2-one (29) in ethanol was added cesium carbonate and dihaloalkane. The reaction mixture was heated to reflux for 4-6 h and was diluted with water and extracted with ethyl acetate. The combined organic layers were washed with brine, dried over sodium sulfate, filtered and concentrated. Purification of the residue by trituration with ethyl acetate/hexanes afforded the title compound.

### 8-(3-Chloropropoxy)-1,3,4,5-tetrahydrobenzo[d][1,3]diazepin-2-one (30)

Following the general alkylation procedure described above, 8-Hydroxy-1,3,4,5-tetrahydrobenzo[d][1,3]diazepin-2-one (29) (0.45 g, 2.5 mmol), 1-bromo-3-chloropropane (1.19 g, 7.58 mmol) and cesium carbonate (1.65 g, 5.10 mmol) in ethanol (40 mL) afforded the title compound (30) as a white solid: ¹H NMR (DMSO-d₆) δ 8.52 (s, 1H), 7.01 (br s, 1H), 6.92 (d, J=8.4 Hz, 1H), 6.64 (d, J=2.4 Hz, 1H), 6.43 (dd, J=8.3, 2.5 Hz, 1H), 3.99 (t, J=6.1 Hz, 2H), 3.77 (t, J=6.5 Hz, 2H), 3.19-3.15 (m, 2H), 2.82 (t, J=4.7 Hz, 2H), 2.18-2.09 (quintet, J=6.2 Hz, 2H); MS (ESI) m/z 254 [C₁₂H₁₅ClN₂O₂+H]⁺.

### 8-(4-Chlorobutoxy)-1,3,4,5-tetrahydrobenzo[d][1,3]diazepin-2-one (31)

Following the general alkylation procedure described above, 8-Hydroxy-1,3,4,5-tetrahydrobenzo[d][1,3]diazepin-2-one (29) (1.20 g, 6.74 mmol), 1-bromo chlorobutane (3.47 g, 20.2 mmol) and cesium carbonate (4.40 g, 13.5 mmol) in ethanol (100 mL) afforded the title compound (31) as a white solid: mp 177-179° C.; ¹H NMR (DMSO-d₆) 88.51 (s, 1H), 7.00 (br s, 1H), 6.90 (d, J=8.4 Hz, 1H), 6.62 (d, J=2.4 Hz, 1H), 6.41 (dd, J=8.3, 2.4 Hz, 1H), 3.90 (t, J=6.0 Hz, 2H), 3.70 (t, J=6.1 Hz, 2H), 3.35-3.15 (m, 2H), 2.83 (t, J=4.7 Hz, 2H), 1.85-1.80 (m, 4H); MS (ESI) m/z 269 [C₁₃H₁₇ClN₂O₂+H]⁺

### Example 27

### General Procedure for Displacement of a Halide with a Dichlorophenyl Piperazine

To a solution of a halide, either of compounds 30 or 31, in acetonitrile was added a dichlorophenyl piperazine hydrochloride, sodium iodide and potassium carbonate. The reaction mixture was heated to reflux for 2 days, cooled to room temperature and diluted with water. The aqueous phase was extracted with ethyl acetate (3×50 mL) and the combined organic layers were dried over sodium sulfate. Solvent removal in vacuo followed by purification of the residue by silica gel chromatography (eluant: 90:10 ethyl acetate/methanol) afforded the desired compound as a white solid.

### Example 28

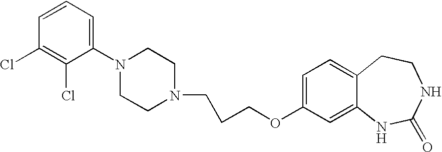

### 8-{3-[4-(2,3-Dichlorophenyl)piperazin-1-yl]propoxy}-1,3,4,5-tetrahydrobenzo[d][1,3]diazepin-2-one

Following the general procedure of Example 27, above, 8-(3-chloropropoxy)-1,3,4,5-tetrahydrobenzo[d][1,3]diazepin-2-one (30) (0.52 g, 2.0 mmol), dichlorophenyl piperazine hydrochloride (0.65 g, 2.5 mmol), sodium iodide (0.61 g, 4.1 mmol) and potassium carbonate (0.85 g, 6.1 mmol) in acetonitrile (60 mL), afforded the title compound as an off-white solid: mp 183-184° C.; ¹H NMR (DMSO-d₆) δ 8.50 (s, 1H), 7.31-7.28 (m, 2H), 7.16-7.13 (m, 1H), 6.99 (br s, 1H), 6.90 (d, J=8.4 Hz, 1H), 6.63 (d, J=2.4 Hz, 1H), 6.41 (dd, J=8.3, 2.4 Hz, 1H), 3.93 (t, J=6.3 Hz, 2H), 3.18-3.15 (m, 2H), 2.99-2.96 (m, 4H), 2.81 (t, J=4.7 Hz, 2H), 2.56-2.54 (m, 2H), 2.50-2.46 (m, 4H), 1.92-1.86 (m, 2H); MS (ESI) m/z 449 [C₂₂H₂₆Cl₂N₄O₂+H]⁺

### Example 29

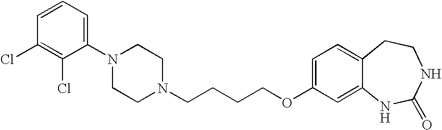

### 8-{4-[4-(2,3-Dichlorophenyl)piperazin-1-yl]butoxy}-1,3,4,5-tetrahydrobenzo[d][1,3]diazepin-2-one

Following the general procedure of Example 27, 8-(4-Chlorobutoxy)-1,3,4,5-tetrahydrobenzo[d][1,3]diazepin-2-one (31) (1.87 g, 6.96 mmol), dichlorophenyl piperazine hydrochloride (2.60 g, 9.75 mmol), sodium iodide (2.10 g, 13.9 mmol) and potassium carbonate (2.90 g, 20.9 mmol) in acetonitrile (100 mL), afforded the title compound as an off-white solid: mp 185-186° C.; ¹H NMR (DMSO-d₆) δ 8.51 (d, J=1.7 Hz, 1H), 7.31-7.29 (m, 2H), 7.14 (dd, J=6.1, 3.5 Hz, 1H), 7.00 (br s, 1H), 1.60-1.57 (m, 2H), 6.90 (d, J=8.4 Hz, 1H), 6.62 (d, J=2.4 Hz, 1H), 6.41 (dd, J=8.3, 2.4 Hz, 1H), 3.90° (t, J=6.2 Hz, 2H), 3.19-3.15 (m, 2H), 2.97-2.95 (m, 4H), 2.81 (t, J=4.7 Hz, 2H), 2.53-2.52 (m, 4H), 2.38 (t, J=6.9 Hz, 2H), 1.74-1.69 (m, 2H); MS (ESI) m/z 463 [C₂₃H₂₈Cl₂N₄O₂+H]⁺

### Example 30

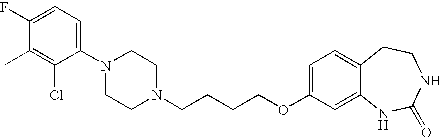

### 8-{4-[4-(2-Chloro-4-fluoro-3-methyl-phenyl)-piperazin-1-yl]-butoxy}-1,3,4,5-tetrahydro-benzo[d][1,3]diazepin-2-one

Following the general procedure of Example 27, 8-(4-chlorobutoxy)-1,3,4,5-tetrahydrobenzo[d][1,3]diazepin-2-one (31) (US Pat App Pub No 20050043309, 0.323 g, 1.20 mmol), 1-(2-chloro-4-fluoro-3-methyl-phenyl)-piperazine (US Pat App Pub No. 20050043309) (0.320 g, 1.20 mmol), and 2M potassium carbonate (1.3 ml, 2.40 mmol) afforded the title compound, MS: APCI: M+1: 461.2 (Exact mass 460.20)

### Example 31

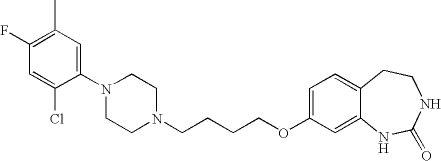

### 8-{4-[4-(2-Chloro-4-fluoro-5-methyl-phenyl)-piperazin-1-yl]-butoxy}-1,3,4,5-tetrahydro-benzo[d][1,3]diazepin-2-one

Following the general procedure of Example 27, 8-(4-chlorobutoxy)-1,3,4,5-tetrahydrobenzo[d][1,3]diazepin-2-one (31) (US Pat App Pub No 20050043309, 0.323 g, 1.20 mmol), and 1-(2-Chloro-4-fluoro-5methyl-phenyl)-piperazine hydrochloride (US Pat App Pub No 20050043309) (0.350 g, 1.20 mmol), and 2M potassium carbonate (1.3 ml, 2.40 mmol) afforded the title compound, MS: APCI: M+1: 461.2.

### Example 32

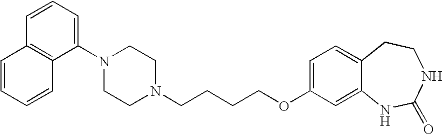

### 8-[4-(4-Naphthalen-1-yl-piperazin-1-yl)-butoxy]-1,3,4,5-tetrahydro-benzo[d][1,3]diazepin-2-one

Following the general procedure of Example 27, 8-(4-chlorobutoxy)-1,3,4,5-tetrahydrobenzo[d][1,3]diazepin-2-one (31) (US Patent Application Publication No. 20050043309, 0.128 g, 1.20 mmol), and 1-naphthalen-1-yl-piperazine (0.152 g, 0.612 mmol), potassium iodide (0.106 g, 0.637 mmol) and potassium carbonate (1.1 g, 0.79 mmol) afforded the title compound recovered as the dihydrochloride salt MS: APCI: M+1: 445.3 (Exact mass 444.57).

### Example 33

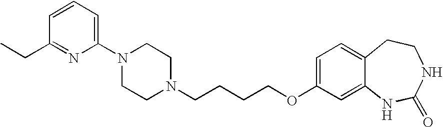

### 8-{(4-[4-(6-Ethyl-pyridin-2-yl)-piperazin-1-yl]-butoxy}-1,3,4,5-tetrahydro-benzo[d][1,3]diazepin-2-one

To a mixture of 1 ml water and 3 ml acetonitrile in each of two sealable microwave tubes was added potassium carbonate (4.96 mmol, 0.685 g), 1-(6-ethyl-pyridin-2-yl)-piperazine (US Pat App Pub No 20050043309, 1.24 mmol, 0.237 g) and 8-(4-chlorobutoxy)-1,3,4,5-tetrahydrobenzo[d][1,3]diazepin-2-one (31) (0.237 g, 1.24 mmol). After heating for 2 hours at 120° C., The mixture was extracted with ethyl acetate and the combined organic layers were dried over magnesium sulfate. Solvent removal in vacuo followed by purification of the residue by silica gel chromatography (eluant: 98:2 dichloromethane/methanol) afforded the title compound as a white foam. MS: APCI: M+1: 424.3 (Exact mass 423.26)

### Example 34

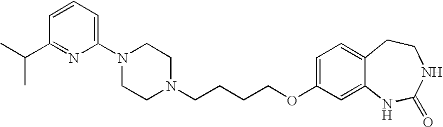

### 8-{4-[4-(6-Isopropyl-pyridin-2-yl)-piperazin-1-yl]-butoxy}-1,3,4,5-tetrahydro-benzo[d][1,3]diazepin-2-one

To a mixture of 1 ml water and 3 ml acetonitrile in each of two sealable microwave tubes was added potassium carbonate (5.2 mmol, 0.72 g), 1-(6-Isopropyl-pyridin-2-yl)-piperazine (US Pat App Pub No. 20050043309, 1.3 mmol, 0.27 g) and 8-(4-Chlorobutoxy)-1,3,4,5-tetrahydrobenzo[d][1,3]diazepin-2-one (31) (0.42 g, 1.56 mmol). After heating for 2 hours at 120° C., The mixture was extracted with ethyl acetate and the combined organic layers were dried over magnesium sulfate. Solvent removal in vacuo followed by purification of the residue by silica gel chromatography (eluant: 98:2 dichloromethane/methanol) afforded the title compound as a white foam. MS: APCI: M+1: 438.2 (Exact mass 437.28)

### Example 35

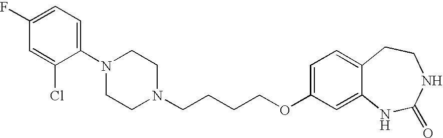

### 8-{4-[4-(2-Chloro-4-fluoro-phenyl)-piperazin-1-yl]-butoxy}-1,3,4,5-tetrahydro-benzo[d][1,3]diazepin-2-one

Following the general procedure of Example 27, 8-(4-chlorobutoxy)-1,3,4,5-tetrahydrobenzo[d][1,3]diazepin-2-one (31) (US Pat App Pub No 20050043309, 0.500 g, 1.86 mmol), and 1-(2-chloro-4-fluorophenyl)-piperazine (US Pat App Pub No 20050043309) (0.52 g, 2.41 mmol), and potassium carbonate (1.03 g, 7.44 mmol) afforded the title compound, MS: APCI: M+1: 447.1 (Exact mass 446.19)

### Example 36

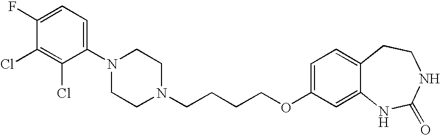

### 8-{4-[4-(2,3-Dichloro-4-fluoro-phenyl)-piperazin-1-yl]-butoxy}-1,3,4,5-tetrahydro-benzo[d][1,3]diazepin-2-one

Following the general procedure of Example 27, 8-(4-chlorobutoxy)-1,3,4,5-tetrahydrobenzo[d][1,3]diazepin-2-one (31) (0.417 g, 1.55 mmol), and 1-(2,3-dichloro-4-fluorophenyl)-piperazine (US Pat App Pub No 20050043309, 0.50 g, 1.55 mmol), and potassium carbonate (1.73 g, 12.4 mmol) afforded the title compound, MS: APCI: M+1: 482.1 (Exact mass 480.15)

### Example 37

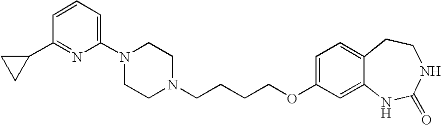

### 8-[4-[4-(6-Cyclopropyl-pyridin-2-yl)-piperazin-1-yl]-butoxy-1,3,4,5-tetrahydro-benzo[d][1,3]diazepin-2-one

To a mixture of 1 ml water and 3 ml acetonitrile in each of three sealable microwave tubes was added potassium carbonate (3.3 mmol, 0.46 g), 1-(6-cyclopropyl-pyridin-2-yl)-piperazine (US Pat App Pub No 20050043309, 90.83 mmol, 0.21 g) and 8-(4-chlorobutoxy)-1,3,4,5-tetrahydrobenzo[d][1,3]diazepin-2-one (31) (0.27 g, 0.99 mmol). After heating for 2 hours at 110° C., The mixture was extracted with ethyl acetate and the combined organic layers were dried over magnesium sulfate. Solvent removal in vacuo followed by purification of the residue by silica gel chromatography (eluant: 98:2 dichloromethane/methanol) afforded the title compound as a white solid, MS: APCI: M+1: 436.2 (Exact mass 435.26) MP: 155-156° C.

### Example 38

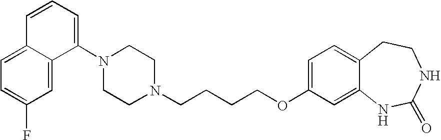

### 8-{4-[4-(7-Fluoro-naphthalen-1-yl)-piperazin-1-yl]-butoxy}-1,3,4,5-tetrahydro-benzo[d][1,3]diazepin-2-one

To a flask containing 8-(4-Chloro-butoxy)-1,3,4,5-tetrahydro-benzo[d][1,3]diazepin-2-one (31) (0.454 g, 1.69 mmol), and 1-(7-fluoro-naphthalen-1-yl)-piperazine TFA salt (US Pat App Pub No 20050043309, 0.612 g, 1.78 mmol), potassium iodide (0.149 g, 0.901 mmol) and aqueous sodium carbonate (2.0 M, 2 mL, 4 mmol) and water (5 mL) was heated at 97° C. for 16 hours. Acetonitrile was added and reaction cooled to room temperature. Silica gel was added and reaction mixture concentrated. Purification by liquid chromatography (LC) (0-5% methanol: Ethyl Acetate) provided an oil which was treated with 1 N HCl in ether to afford the title compound as the hydrochloride salt, MS: APCI: M+1: 463.3 (Exact mass 462.5).

### Example 39

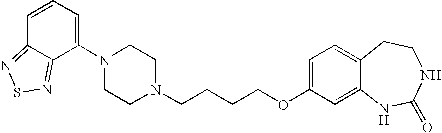

### 8-{4-[4-(2,1,3-benzothiadiazol-4-yl)piperazin-1-yl]butoxy}-1,3,4,5-tetrahydro-2H-1,3-benzodiazepin-2-one

To a flask containing 8-(4-Chloro-butoxy)-1,3,4,5-tetrahydro-benzo[d][1,3]diazepin-2-one (31) (US Pat App Pub No 20050043309, 0.238 g, 0.887 mmol), and 4-piperazin-1-yl-benzo[2,1,3]thiadiazole HCl salt (0.203 g, 0.793 mmol), potassium iodide (0.0814 g, 0.490 mmol) and aqueous sodium carbonate (2.0 M, 0.8 mL, 2 mmol) and water (5 mL) was heated at 97° C. for 16 hours. Acetonitrile was added and reaction cooled to room temperature. Silica gel was added and reaction mixture concentrated. Purification by LC (0-5% methanol: Ethyl Acetate) provided an oil which was treated with 1 N HCl in ether to afford the title compound which was recovered as the hydrochloride salt, MS: APCI: M+1: 453.3 (Exact mass 452.5)

### Example 40

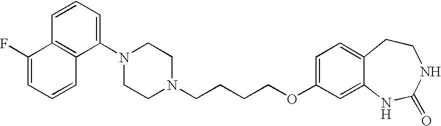

### 8-{4-[4-(5-Fluoro-naphthalen-1-yl)-piperazin-1-yl]-butoxy}-1,3,4,5-tetrahydro-benzo[d][1,3]diazepin-2-one

To a flask containing 8-(4-chloro-butoxy)-1,3,4,5-tetrahydro-benzo[d][1,3]diazepin-2-one (31) (0.287 g, 1.06 mmol), and 1-(5-fluoro-naphthalen-1-yl)-piperazine HCl salt (US Pat App Pub No 20050043309, 0.269 g, 1.01 mmol), potassium iodide (0.102 g, 0.616 mmol) and aqueous sodium carbonate (2.0 M, 1.0 mL, 2 mmol) and water (5 mL) was heated at 97° C. for 16 hours. Acetonitrile was added and reaction cooled to room temperature. Silica gel was added and reaction mixture concentrated. Purification by LC (0-5% methanol: Ethyl Acetate) provided the an oil which was treated with 1 N HCl in ether to afford the title compound as the hydrochloride salt, MS: APCI: M+1: 463.3 (Exact mass 462.5)

### Example 41

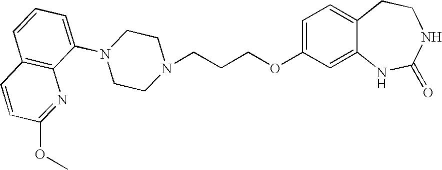

### 8-{3-[4-(2-Methoxy-quinolin-8-yl)-piperazin-1-yl]-propoxy}-1,3,4,5-tetrahydro-benzo[d][1,3]diazepin-2-one

To a flask containing 8-(3-Chloro-propoxy)-1,3,4,5-tetrahydro-benzo[d][1,3]diazepin-2-one (30) (US Pat App Pub No 20050043309, 0.1875 g, 0.736 mmol), and 2-Methoxy-8-piperazin-1-yl-quinoline TFA salt (0.251 g, 0.704 mmol), potassium iodide (0.070 g, 0.422 mmol) and aqueous sodium carbonate (2.0 M, 0.37 mL, 0.74 mmol) and water (5 mL) was heated at 97° C. for 16 hours. Acetonitrile was added and reaction cooled to room temperature. Silica gel was added and reaction mixture concentrated. Purification by LC (0-5% methanol: Ethyl Acetate) provided the desired product as an oil which treated with 1 N HCl in ether to afford the title compound as the hydrochloride salt MS: APCI: M+1: 462.1 (Exact mass 461.5)

### Example 42

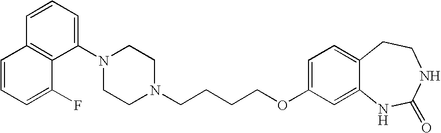

### 8-{4-[4-(8-Fluoro-naphthalen-1-yl)-piperazin-1-yl]-butoxy}-1,3,4,5-tetrahydro-benzo[d][1,3]diazepin-2-one

To a flask containing 8-(4-chloro-butoxy)-1,3,4,5-tetrahydro-benzo[d][1,3]diazepin-2-one (31) (0.323 g, 1.2 mmol), and 1-(8-fluoro-naphthalen-1-yl)-piperazine HCl salt (US Pat App Pub No 20050043309, 0.419 g, 1.30 mmol), potassium iodide (0.120 g, 0.725 mmol) and aqueous sodium carbonate (2.0 M, 0.6 mL, 1 mmol) and water (5 mL) was heated at 97° C. for 16 hours. Acetonitrile was added and reaction cooled to room temperature. Silica gel was added and reaction mixture concentrated. Purification by LC (0-5% methanol: Ethyl Acetate) provided the title compound as a solid MS: APCI: M+1: 463.3 (Exact mass 462.5)

### Example 43

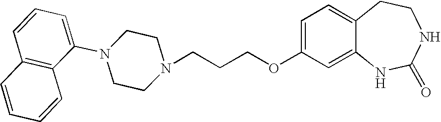

### 8-[3-(4-Naphthalen-1-yl-piperazin-1-yl)-propoxy]-1,3,4,5-tetrahydro-benzo[d][1,3]diazepin-2-one

To a flask containing 8-(3-chloro-propoxy)-1,3,4,5-tetrahydro-benzo[d][1,3]diazepin-2-one (30) (US Pat App Pub No 20050043309, 0.334 g, 1.31 mmol), and 1-naphthalen-1-yl-piperazine HCl salt (0.324 g, 1.30 mmol), potassium iodide (0.103 g, 0.619 mmol) and aqueous sodium carbonate (2.0 M, 1.8 mL, 3.6 mmol) and water (5 mL) was heated at 97° C. for 16 hours. Acetonitrile was added and reaction cooled to room temperature. Silica gel was added and reaction mixture concentrated. Purification by LC (0-5% methanol: Ethyl Acetate) provided the title compound as a solid, MS: APCI: M+1: 431.1 (Exact mass 430.5)

### Example 44

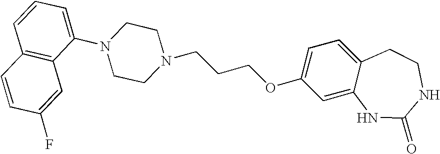

### 8-{3-[4-(7-Fluoro-naphthalen-1-yl)-piperazin-1-yl]-propoxy}-1,3,4,5-tetrahydro-benzo[d][1,3]diazepin-2-one

To a flask containing 8-(3-chloro-propoxy)-1,3,4,5-tetrahydro-benzo[d][1,3]diazepin-2-one (30) (0.321 g, 1.256 mmol), and 1-(7-fluoro-naphthalen-1-yl)-piperazine TFA salt (US Pat App Pub No 20050043309, 0.446 g, 1.30 mmol), potassium iodide (0.110 g, 0.668 mmol) and aqueous sodium carbonate (2.0 M, 2.0 mL, 1 mmol) and water (5 mL) was heated at 97° C. for 16 hours. Acetonitrile was added and reaction cooled to room temperature. Silica gel was added and reaction mixture concentrated. Purification by LC (0-5% methanol: Ethyl Acetate) provided the title compound as a solid, MS: APCI: M+1: 449.1 (Exact mass 448.5).

### Example 45

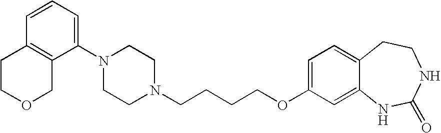

### 8-[4-(4-Isochroman-8-yl-piperazin-1-yl)-butoxy]-1,3,4,5-tetrahydro-benzo[d][1,3]diazepin-2-one

To a flask containing 8-(4-chlorobutoxy)-1,3,4,5-tetrahydrobenzo[d][1,3]diazepin-2-one (31) (0.128 g, 1.20 mmol), and 1-isochroman-8-yl-piperazine (US Pat App Pub No 20050043309, 0.152 g, 0.612 mmol) aqueous sodium carbonate (2.0 M, 0.435 mL, 0.870 mmol) and water (4 mL) was heated at 95° C. for ˜2 hours. 4 mL of acetonitrile was added and the reaction was heated overnight hour at ˜80° C. A steam of nitrogen was blown over the reaction to reduce the volume to ˜4 mL. To the crude mixture was CH₂Cl₂ and water and the layers were separated. To the collected organics was added silica gel and the solvents were removed in vacuo. Purification by LC (1-8% Methanol w/10% NH₄OH (based on amount of MeOH) in CH₂Cl₂, AnaLogix, RS-40) afforded the title compound as an off white solid, MS: APCI: M+1: 451.1 (450.3).

### Example 46

### 7-Bromo-4,4-dimethyl-3,4-dihydro-2H-naphthalen-1-one Oxime (33)

A mixture of 7-bromo-4,4-dimethyl-3,4-dihydro-2H-naphthalen-1-one 32 (6.50 g, 25.7 mmol, Endo, Y. et al. J. Med. Chem. 1998, 41, 1476-1496.), hydroxylamine hydrochloride (2.16 g, 31.1 mmol), and sodium acetate (4.21 g, 51.3 mmol) in ethanol (38 mL) and water (38 mL) was heated to reflux for 14 h. After cooling to room temperature, dichloromethane (100 mL) was added, and the layers were separated. The aqueous layer was extracted with dichloromethane (2×50 mL), and the combined organic layers were washed with a saturated sodium bicarbonate solution (3×50 mL) and brine, dried over sodium sulfate, filtered and concentrated at reduced pressure to afford the title compound (33) as a brown oil: ¹H NMR (CDCl₃) δ 8.05 (d, J=2.2 Hz, 1H), 7.42 (dd, J=8.4, 2.2 Hz, 1H), 7.23 (d, J=8.4 Hz, 1H), 2.84 (t, J=6.9 Hz, 2H), 1.73 (t, J=6.9 Hz, 2H), 1.28 (s, 6H); MS (ESI) m/z 269 [C₁₂H₁₄BrNO+H]⁺.

### 8-Bromo-5,5-dimethyl-1,3,4,5-tetrahydrobenzo[b]azepin-2-one (34)

To polyphosphoric acid (150 mL) heated to 110° C. with an oil bath and stirred with an overhead stirrer was added a solution of 7-bromo-4,4-dimethyl-3,4-dihydro-2H-naphthalen-1-one Oxime (33) (6.50 g, 24.2 mmol) in dichloromethane (10 mL) via syringe over 5 min. The dichloromethane was removed by distillation, and the residual mixture was heated at 110-120° C. for 10 min and quickly poured into ice water (1.5 L). After stirring for 1 h, the resulting precipitate was collected by filtration. The filtrate was extracted with dichloromethane (2×200 mL), and the organic layers were combined, washed with a saturated sodium bicarbonate solution (200 mL) and brine (100 mL), dried over sodium sulfate, filtered and concentrated at reduced pressure. The residue was combined with the collected solid for chromatography (silica gel flash column, 75:25 hexanes/ethyl acetate) to afford the title compound (33) as a tan solid: ¹H NMR (CDCl₃) δ 7.89 (br s, 1H), 7.27-7.26 (m, 2H), 7.11 (s, 1H), 2.39 (t, J=7.0 Hz, 2H), 2.10 (t, J=7.0 Hz, 2H), 1.39 (s, 6H); MS (ESI) m/z 269 [C₁₂H₁₄BrNO+H]⁺.

### 8-Hydroxy-5,5-dimethyl-1,3,4,5-tetrahydro-benzo[b]azepin-2-one (35)

A solution of 8-bromo-5,5-dimethyl-1,3,4,5-tetrahydrobenzo[b]-azepin-2-one (34) (1.50 g, 5.60 mmol) in tetrahydrofuran (15 mL) was cooled to −78° C. and N,N,N′,N′-tetramethylethylenediamine (3.60 mL, 23.5 mmol) was added followed by n-butyllithium (14.0 mL, 22.4 mmol, 1.6 M solution in hexanes). After stirring for 1 h at 78° C., trimethyl borate (2.90 g, 28.0 mmol) was added and the reaction mixture was allowed to warm to room temperature. After another 1 h, water (10 mL) was carefully added, stirred for 10 minutes, followed by the addition of aqueous hydrogen peroxide (30%, 9 mL) and the solution was stirred at room temperature overnight. The reaction was quenched by the slow addition of solid sodium bisulfite and the mixture extracted with ethyl acetate (3×50 mL). The organic layers were combined, washed with a 1 M sodium hydroxide solution (3×40 mL) and the organic layer discarded. The aqueous layers were combined, acidified with 1 M hydrochloric acid to pH 1-2 and extracted with ethyl acetate (3×75 mL). The organic layers were combined, washed with brine, dried over sodium sulfate, filtered and concentrated to afford the desired product, title compound (35), as a tan solid: ¹H NMR (CD₃OD) δ 7.23 (d, J=8.5 Hz, 1H), 6.60 (dd, J=8.6, 1.4 Hz, 1H), 6.47 (d, J=1.6 Hz, 1H), 2.28 (t, J=7.0 Hz, 2H), 2.05 (t, J=7.0 Hz, 2H), 1.34 (s, 6H); MS (ESI) m/z 206 [C₁₂H₁₅NO₂+H]⁺.

### Example 48

### General procedure for alkoxylation of a 8-Hydroxy-tetrahydrobenzo[b]azepin-2-one with a dihaloalkane

To a stirred solution of 8-Hydroxy-5,5-dimethyl-1,3,4,5-tetrahydro-benzo[b]azepin-2-one (35) in ethanol was added cesium carbonate followed by a dihaloalkane. After stirring at 55° C. for 4 to 24 h, the reaction mixture was diluted with water and extracted with ethyl acetate. The combined organic layers were washed with brine, dried over sodium sulfate, filtered and concentrated. Purification by silica gel chromatography afforded the title compound.

### Example 49

### 8-(3-Bromopropoxy)-5,5-dimethyl-1,3,4,5-tetrahydrobenzo[b]azepin-2-one (36)

Following the general procedure of Example 48, 8-hydroxy-5,5-dimethyl-1,3,4,5-tetrahydro-benzo[b]azepin-2-one (35) (0.50 g, 2.44 mmol), 1,3-dibromopropane (0.98 g, 4.9 mmol) and cesium carbonate (1.19 g, 3.65 mmol) in ethanol (8 mL) afforded an inseparable mixture (3:2) of the title compound (36) and by-product olefin respectively as a white solid: ¹H NMR (CDCl₃) δ 7.32-7.28 (m, 2H), 7.21 (br s, 1H), 6.71 (dd, J=7.2, 1.4 Hz, 1H), 6.46 (d, J=1.4 Hz, 1H), 6.08-5.99 (m, 0.3H), 5.38-5.28 (m, 0.7H), 4.53-4.52 (m, 0.6H), 4.14 (t, J=7.3 Hz, 1.2H), 3.60 (t, J=6.4 Hz, 1.2H), 2.41-2.27 (m, 3H), 2.07 (t, J=7.2 Hz, 2H), 1.38 (s, 6H).

### Example 50

### 8-(4-Bromobutoxy)-5,5-dimethyl-1,3,4,5-tetrahydrobenzo[b]azepin-2-one (37)

Following the general procedure of Example 48, 8-hydroxy-5,5-dimethyl-1,3,4,5-tetrahydro-benzo[b]azepin-2-one (35) (0.42 g, 2.1 mmol), 1,4-dibromobutane (0.89 g, 4.1 mmol) and cesium carbonate (1.00 g, 3.08 mmol) in ethanol (5 mL) afforded the title compound (37) as a white solid: ¹H NMR (CDCl₃) δ 7.29 (d, J=8.4 Hz, 1H), 7.21 (br s, 1H), 6.68 (dd, J=8.8, 2.7 Hz, 1H), 6.43 (d, J=2.7 Hz, 1H), 3.97 (t, J=5.9 Hz, 2H), 3.49 (t, J=6.5 Hz, 2H), 2.37 (t, J=7.1 Hz, 2H), 2.10-1.97 (m, 6H), 1.38 (s, 6H); MS (ESI) m/z 340 [C₁₆H₂₂BrNO₂+H]⁺.

### Example 51

### 8-(5-Bromopentyloxy)-5,5-dimethyl-1,3,4,5-tetrahydrobenzo[b]azepin-2-one (38)

Following the general procedure of Example 48, 8-hydroxy-5,5-dimethyl-1,3,4,5-tetrahydro-benzo[b]azepin-2-one (35) (0.39 g, 1.9 mmol), 1,5-dibromopentane (1.09 g, 4.75 mmol) and cesium carbonate (1.24 g, 3.80 mmol) in ethanol (15 mL) afforded the title compound (38) as a brown liquid: ¹H NMR (CDCl₃) δ 7.30 (d, J=8.8 Hz, 1H), 7.13 (br s, 1H), 6.68 (dd, J=8.7, 2.7 Hz, 1H), 6.42 (d, J=2.6 Hz, 1H), 3.94 (t, J=6.2 Hz, 2H), 3.44 (t, J=6.8 Hz, 2H), 2.38 (t, J=7.0 Hz, 2H), 2.07 (t, J=7.2 Hz, 2H), 1.96-1.76 (m, 4H), 1.67-1.60 (m, 2H), 1.38 (s, 6H); MS (ESI) m/z 354 [C₁₇H₂₄BrNO₂+H]⁺.

### Example 52

### General procedure for displacement of a halide with 2,3-dichlorophenyl piperazine hydrochloride

To a solution of a halide, any one of compounds 36-38, in acetonitrile was added 2,3-dichlorophenyl piperazine hydrochloride, sodium iodide and potassium carbonate. The reaction mixture was heated to reflux for times varying from 3 h to 3 d, the mixture was cooled and diluted with water. The aqueous suspension was extracted with methylene chloride (2×) and the organic layers were combined, dried over sodium sulfate, filtered and concentrated. Purification by silica gel chromatography afforded the desired product.

### Example 53

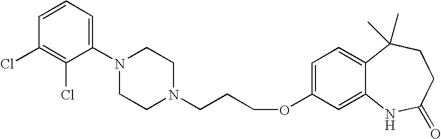

### 8-{3-[4-(2,3-Dichlorophenyl)piperazin-1-yl]-propoxy}-5,5-dimethyl-1,3,4,5-tetrahydrobenzo[b]azepin-2-one

Following the general procedure of Example 52, 8-(3-bromopropoxy)-5,5-dimethyl-1,3,4,5-tetrahydrobenzo[b]azepin-2-one (36) (0.45 g, 0.83 mmol), 2,3-dichlorophenyl piperazine hydrochloride (0.27 g, 0.99 mmol), sodium iodide (0.15 g, 0.99 mmol) and potassium carbonate (0.34 g, 2.5 mmol) in acetonitrile (25 mL), afforded the title compound as a white solid: mp 162-164° C.; ¹H NMR (CDCl₃) δ 7.30 (d, J=8.7 Hz, 1H), 7.14-7.16 (m, 3H), 6.96 (dd, J=6.2, 3.5 Hz, 1H), 6.71 (dd, J=8.7, 2.6 Hz, 1H), 6.45 (d, J=2.6 Hz, 1H), 4.02 (t, J=6.2 Hz, 2H), 3.08 (br s, 4H), 2.68 (br s, 4H), 2.60 (t, J=7.3 Hz, 2H), 2.38 (t, J=7.1 Hz, 2H), 2.07 (t, J=6.9 Hz, 2H), 2.0 (t, J=7.1 Hz, 2H), 1.38 (s, 6H); MS (ESI) m/z 476 [C₂₅H₃₁Cl₂N₃O₂+H]⁺.

### Example 54

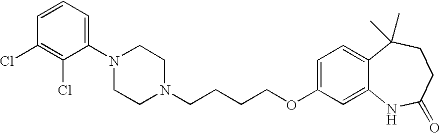

### 8-{4-[4-(2,3-Dichlorophenyl)piperazin-1-yl]-butoxy}-5,5-dimethyl-1,3,4,5-tetrahydrobenzo[b]azepin-2-one

Following the general procedure of Example 52, 8-(4-bromobutoxy)-5,5-dimethyl-1,3,4,5-tetrahydrobenzo[b]-azepin-2-one (37) (0.36 g, 1.1 mmol), 2,3-dichlorophenyl piperazine hydrochloride (0.34 g, 1.3 mmol), sodium iodide (0.19 g, 1.3 mmol) and potassium carbonate (0.44 g, 3.2 mmol) in acetonitrile (25 mL), afforded the title compound as an off-white solid: mp 106-108° C.; ¹H NMR (CDCl₃) δ 7.51 (s, 1H), 7.29 (d, J=8.8 Hz, 1H), 7.15-7.14 (m, 2H), 6.95 (dd, J=6.1, 3.6 Hz, 1H), 6.69 (dd, J=8.7, 3.7 Hz, 1H), 6.45 (d, J=2.6 Hz, 1H), 3.97 (t, J=6.1 Hz, 2H), 3.08 (br s, 4H), 2.66 (br s, 4H), 2.48 (t, J=7.5 Hz, 2H), 2.38 (t, J=7.0 Hz, 2H), 2.07 (t, J=7.4 Hz, 2H), 1.69-1.85 (m, 4H), 1.38 (s, 6H); MS (ESI) m/z 490 [C₂₆H₃₃Cl₂N₃O₂+H]⁺.

### Example 55

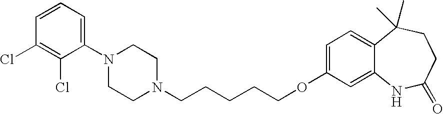

### 8-{5-[4-(2,3-Dichlorophenyl)piperazin-1-yl]-pentyloxy}-5,5-dimethyl-1,3,4,5-tetrahydrobenzo[b]azepin-2-one

Following the general procedure of Example 52, 8-(5-bromopentyloxy)-5,5-dimethyl-1,3,4,5-tetrahydrobenzo[b]azepin-2-one (38) (0.40 g, 1.1 mmol), 2,3-dichlorophenyl piperazine hydrochloride (0.36 g, 1.4 mmol), sodium iodide (0.20 g, 1.4 mmol) and potassium carbonate (0.47 g, 3.4 mmol) in acetonitrile (20 mL), afforded the title compound as a white solid: mp 128-130° C.; ¹H NMR (CDCl₃) δ 7.31-7.28 (m, 2H), 7.17-7.11 (m, 2H), 6.96 (dd, J=6.1, 3.5 Hz, 1H), 6.67 (dd, J=8.7, 2.6 Hz, 1H); 6.43 (d, J=2.6 Hz, 1H), 3.94 (t, J=6.7 Hz, 2H), 3.08 (br s, 4H), 2.65 (br s, 4H), 2.47-2.36 (m, 4H), 2.07 (t, J=7.3 Hz, 2H), 1.81-1.79 (m, 2H), 1.59-1.48 (m, 4H), 1.38 (s, 6H); MS (ESI) m/z 504 [C₂₇H₃₅Cl₂N₃O₂+H]⁺.

### Example 56

### 8-Methoxy-3,3-dimethyl-1,3,4,5-tetrahydrobenzo[b]azepin-2-one (40)

To a stirred solution of 7-methoxy-2,2-dimethyl-3,4-dihydro-2H-naphthalene-1-one (39) (2.00 g, 10.4 mmol) (Beilstein Registry Number 3091415; CAS Registry Number 21568-66-1; Klemm, L. H. et al. J. Org. Chem. 1968, 33, 1480-1488) in pyridine (50 mL) was added hydroxylamine hydrochloride (2.17 g, 31.2 mmol) and the mixture was heated at 80° C. for 16 h. After cooling to room temperature, the reaction mixture was concentrated, water (75 mL) was added to the residue and the mixture was extracted with dichloromethane (3×100 mL). The organic layers were combined, washed with brine (100 mL), dried over sodium sulfate and concentrated. The crude oxime was then added to polyphosphoric acid (50 mL) at 115° C. and stirred for 5 min. The hot mixture was poured into ice/water and stirred overnight. The precipitated solids were filtered, washed with water and dried to afford the title compound (40) as an off-white solid: ¹H NMR (CD₃OD) δ 7.08 (d, J=8.4 Hz, 1H), 6.64 (dd, J=8.3, 2.6 Hz, 1H), 6.53 (d, J=2.5 Hz, 1H), 3.8 (s, 3H), 2.73 (t, J=6.6 Hz, 2H), 1.99 (t, J=6.8 Hz, 2H), 1.03 (s, 6H); MS (ESI) m/z 220 [C₁₃H₁₇NO₂+H]⁺.

### 8-Hydroxy-3,3-dimethyl-1,3,4,5-tetrahydrobenzo[b]azepin-2-one (41)

A solution of 8-Methoxy-3,3-dimethyl-1,3,4,5-tetrahydrobenzo[b]azepin-2-one (40) (1.0 g, 4.6 mmol) in dichloromethane (60 mL) was cooled to −78° C. and to it, boron tribromide (10 mL, 1.0 M solution in dichloromethane) was added. The reaction mixture was allowed to warm to room temperature and stir overnight. Ether (5 mL) was added and the mixture was poured into an ice/water (30 g) mixture, stirred for 2 h and the precipitated solids were filtered, washed with water and dried to afford the title compound (41) as a tan solid: ¹H NMR (CDCl₃) δ 7.3 (s, 1H), 7.01 (d, J=2.2 Hz, 1H), 6.56 (dd, J=8.2, 1.5 Hz, 1H), 6.39 (d, J=2.4 Hz, 1H), 5.34 (br s, 1H), 2.75 (t, J=6.5 Hz, 2H), 1.98 (t, J=6.5 Hz, 2H), 1.10 (s, 6H); MS (ESI) m/z 206 [C₁₂H₁₅NO₂+H]⁺.

### 8-(4-Chlorobutoxy)-3,3-dimethyl-1,3,4,5-tetrahydrobenzo[b]azepin-2-one (42)

Following the general procedure of Example 48, using 8-Hydroxy-3,3-dimethyl-1,3,4,5-tetrahydrobenzo[b]azepin-2-one (41) (0.30 g, 1.5 mmol), 1-bromo-4-chlorobutane (0.50 g, 2.9 mmol) and cesium carbonate (0.70 g, 2.2 mmol) in ethanol (5 mL) afforded the title compound (42) as a white solid: ¹H NMR (CDCl₃) δ 7.62 (s, 1H), 7.05 (d, J=8.3 Hz, 1H), 6.60 (dd, J=8.3, 2.5 Hz, 1H), 6.39 (d, J=2.5 Hz, 1H), 3.95 (t, J=5.7 Hz, 2H), 3.62 (t, J=6.2 Hz, 2H), 2.76 (t, J=6.6 Hz, 2H), 2.02-1.91 (m, 6H), 1.10 (s, 6H); MS (ESI) m/z 296 [C₁₆H₂₂ClNO₂+H]⁺.

### Example 57

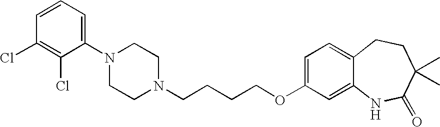

### 8-{4-[4-(2,3-Dichlorophenyl)piperazin-1-yl]butoxy}-3,3-dimethyl-1,3,4,5-tetrahydrobenzo[b]azepin-2-one

Following the general procedure of Example 52, using 8-(4-Chlorobutoxy)-3,3-dimethyl-1,3,4,5-tetrahydrobenzo[b]-azepin-2-one (42) (0.16 g, 0.54 mmol), 2,3-dichlorophenyl piperazine hydrochloride (0.17 g, 0.65 mmol), sodium iodide (0.10 g, 0.65 mmol) and potassium carbonate (0.22 g, 1.6 mmol) in acetonitrile (15 mL) afforded the title compound as a white solid: mp 110-112° C.; ¹H NMR (CDCl₃) δ 7.18-7.14 (m, 3H), 7.05 (d, J=8.3 Hz, 1H), 6.97-6.94 (dd, J=6.0, 3.5 Hz, 1H), 6.62-6.60 (dd, J=8.4, 2.4 Hz, 1H), 6.39 (d, J=2.4 Hz, 1H), 3.95 (t, J=6.1 Hz, 2H), 3.07 (br s, 4H), 2.76 (t, J=6.6 Hz, 2H), 2.66 (br s, 4H), 2.46 (t, J=7.4 Hz, 2H), 1.99 (t, J=6.9 Hz, 2H), 1.85-1.66 (m, 4H), 1.58 (s, 6H); MS (ESI) m/z 490 [C₂₆H₃₃Cl₂N₃O₂+H]⁺.

### Example 58

### 4-Methoxy-1-methyl-2-nitro-benzene (44)

To a solution of 4-methyl-3-nitro-phenol (43) (6.12 g, 40 mmol) in DMSO (40 mL) was added NaOH (2.4 g, 60 mmol) and MeI (3.75 mL, 60 mmol). The mixture thus obtained was stirred at RT for 16 h. Water (100 mL) was added to quench the reaction. The mixture was extracted with EtOAc (250 mL). The organic phase was washed with water (2×100 mL) and brine (50 mL), dried and concentrated to give the title compound (44) which was used in the next step without further purification. ¹HNMR (400 MHz, CDCl₃): δ 7.50 (m, 1H), 7.22 (d, 1H), 7.05 (m, 1H), 3.85 (s, 3H), 2.58 (s, 3H).

### 1-Bromomethyl-4-methoxy-2-nitro-benzene (45)

A mixture of 4-methoxy-1-methyl-2-nitro-benzene (44), (6.7 g, 40 mmol), NBS (8.54 g, 48 mmol) and benzoyl peroxide (0.48 g, 2 mmol) in CCl₄ (50 mL) was refluxed for 16 h, cooled to RT, diluted with hexanes (200 mL), filtered through a pad of celite. The filtrate was concentrated to give the title compound (45), which was used in the next step without further purification ¹HNMR (400 MHz, CDCl₃): δ 7.60 (d, 1H), 7.50 (d, 1H), 7.15 (dd, 1H), 4.80 (s, 2H), 3.90 (s, 3H).

### Acetic acid 4-Methoxy-2-nitro-benzyl ester (46)

To a solution of compound 1-bromomethyl-4-methoxy-2-nitro-benzene (45), in DMF (60 mL) was added NaOAc (16.4 g, 0.2 mol). The mixture was heated at 80° C. for 3 h, cooled to RT, diluted with H₂O (100 mL) extracted with EtOAc (200 mL). The organic phase was washed with H₂O (2×100 mL) and brine (100 mL), dried and concentrated. The residue was purified by chromatography on silica gel to give the title compound (46), in three steps. ¹HNMR (400 MHz, CDCl₃): δ 7.65 (d, 1H), 7.50 (d, 1H), 7.20 (dd, 1H), 5.40 (s, 2H), 3.80 (s, 3H), 2.20 (s, 3H).

### (4-Methoxy-2-nitro-phenyl)-methanol (47)

To a solution of acetic acid 4-methoxy-2-nitro-benzyl ester (46), (7.23 g, 32.4 mmol) in MeOH (30 mL) was added MeOH (5.25 g, 97.3 mmol) in portions. After the addition was over, the mixture was stirred at RT for 3 h. It was then diluted with EtOAc (200 mL) and washed with H₂O (2×50 mL) and brine, dried and concentrated. The residue was purified by chromatography on silica gel to give the title compound (47), ¹HNMR (400 MHz, CDCl₃): δ 7.60 (m, 2H), 7.20 (d, 1H), 4.90 (s, 2H), 3.90 (s, 3H), 2.60 (br s, 1H).

### 1-chloromethyl-4-methoxy-2-nitro-benzene (48)

To a solution of (4-Methoxy-2-nitro-phenyl)-methanol (47), (4.48 g, 24.5 mmol) in CHCl₃ (100 mL) was added PCl₅ (5.88 g, 28.2 mmol) in portions. After the addition was over, the mixture was stirred at RT for 1 h. It was poured into ice-water (100 mL). The mixture was extracted with CHCl₃ (100 mL). The organic phase was washed with brine (50 mL), dried and concentrated to give the title compound (48). ¹HNMR (400 MHz, CDCl₃): δ 7.60 (m, 2H), 7.20 (dd, 1H), 4.95 (s, 2H), 3.90 (s, 2H).

### 4-Methoxy-1-(2-methyl-2-nitro-propyl)-2-nitro-benzene (49)

To a solution of 1-chloromethyl-4-methoxy-2-nitro-benzene (48), (1.31 g, 6.5 mmol) in HMPA (10 mL) was added compound lithium 2-nitropropane (3.09 g, 32.5 mmol) in one portion. The mixture was stirred at RT for 1 h. The reaction was quenched with ice-water (20 mL). The mixture was extracted with EtOAc (50 mL). The organic phase was washed with 1 N HCl (30 mL), H₂O (20 mL) and brine (30 mL), dried and concentrated. The residue was purified by chromatography on silica gel to give the title compound 49, (0.82 g, 50%). ¹HNMR (400 MHz, CDCl₃): δ 7.45 (d, 1H), 7.10 (m, 2H), 3.90 (s, 3H), 3.61 (s, 2H), 1.60 (s, 6H).

### 2-(2-Amino-2-methyl-propyl)-5-methoxy-phenylamine (50)

A mixture of 4-Methoxy-1-(2-methyl-2-nitro-propyl)-2-nitro-benzene (49), (0.82 g, 3.23 mmol) and Raney Nickel (0.5 g) in MeOH was hydrogenated under 50 psi for 3 h. It was then filtered through a pad of celite. The filtrate was concentrated to give the title compound 50, which was used in the next step without further purification. ¹HNMR (400 MHz, CDCl₃): δ 6.90 (d, 1H), 6.40 (d, 1H), 6.30 (dd, 1H), 3.70 (s, 3H), 2.60 (s, 2H), 1.20 (s, 6H).

### 8-Methoxy-4,4-dimethyl-1,3,4,5-tetrahydro-benzo[d][1,3]diazepin-2-one (51)

To a solution of 2-(2-Amino-2-methyl-propyl)-5-methoxy-phenylamine (50), (0.62 g, 3.2 mmol) obtained in last step in THF (50 mL) was added carbonyldiimidazole (CDI) (0.55 g, 3.4 mmol). The mixture was refluxed for 16 h, cooled to RT, diluted with EtOAc (150 mL) and washed with 1 N HCl (20 mL) and brine (10 mL), dried and concentrated to give the title compound (51), ¹HNMR (400 MHz, CDCl₃): δ 7.00 (m, 2H), 6.60 (m, 1H), 6.40 (d, 1H), 5.20 (s, 1H), 3.80 (s, 3H), 2.90 (s, 2H), 1.20 (s, 6H).

### 8-Hydroxy-4,4-dimethyl-1,3,4,5-tetrahydro-benzo[d][1,3]diazepin-2-one (52)

To a cooled (−78° C.) solution of 8-Methoxy-4,4-dimethyl-1,3,4,5-tetrahydro-benzo[d][1,3]diazepin-2-one (51), (0.40 g, 1.8 mmol) in dichloromethane (30 mL) was added BBr₃ (0.38 mL) dropwise. After the addition was over, the mixture was stirred at RT for 4 h. Ether was added (50 mL) and the mixture was stirred at RT for 10 min. The solid was collected, washed with ether, dried under high vacuum to give the title compound (52), which was used in the next step without further purification. ¹HNMR (400 MHz, DMSO-d₆): δ 8.55 (s, 1H), 7.80 (d, 1H), 6.55 (s, 1H), 6.40 (s, 1H), 6.30 (m, 1H), 2.70 (s, 2H), 1.10 (s, 6H).

### 8-(4-Chloro-butoxy)-4,4-dimethyl-1,3,4,5-tetrahydrobenzo[d][1,3]-diazepin-2-one (53)

To a solution of 8-Hydroxy-4,4-dimethyl-1,3,4,5-tetrahydro-benzo[d][1,3]diazepin-2-one (52), (0.18 g, 0.87 mmol) in DMSO (10 mL) was added NaOH (87 mg, 2.15 mmol) and 1-bromo-4-chlorobutane (75 mg, 0.43 mmol). The mixture was stirred at RT for 16 h. Water (20 mL) was added. The solid thus formed was collected by filtration and washed with H₂O, hexane and a small volume of ether to give the title compound (53), which was used in the next step without further purification. ¹HNMR (400 MHz, CD₃OD): δ 7.00 (m, 1H), 6.60 (m, 2H), 4.00 (t, 2H), 3.65 (m, 3H), 2.65 (s, 2H), 2.00 (m, 4H).

### 8-(3-Chloro-propoxy)-4,4-dimethyl-1,3,4,5-tetrahydrobenzo[d][1,3]-diazepin-2-one (54)

In a process similar to that used to produce compound (53), above, a solution of 8-Hydroxy-4,4-dimethyl-1,3,4,5-tetrahydro-benzo[d][1,3]diazepin-2-one (52), (0.32 g, 1.5 mmol) in DMSO (10 mL) was added NaOH (90 mg, 2.25 mmol) and 1-bromo-3-chloropropane (165 mg, 1.05 mmol). The mixture was stirred at RT for 16 h. Water (20 mL) was added. The solid thus formed was collected by filtration and washed with H₂O, hexane and a small volume of ether to give the title compound (54), which was used in the next Example, without further purification.

### Example 59

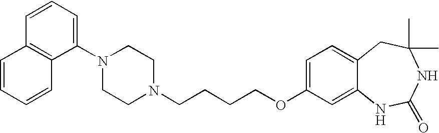

### 4,4-Dimethyl-8-[4-(4-naphthalen-1-yl-piperazin-1-yl)-butoxy]-1,3,4,5-tetrahydro-benzo[d][1,3]diazepin-2-one

A mixture of 8-(4-chloro-butoxy)-4,4-dimethyl-1,3,4,5-tetrahydro-benzo[d][1,3]diazepin-2-one 53, (0.1 g, 0.34 mmol), 1-naphthalen-1-yl-piperazine hydrochloride (US Pat App Pub No 20050043309, 0.1 g, 0.34 (86 mg, 0.41 mmol), NaI (0.10 g, 0.68 mmol) and K₂CO₃ (0.14 g, 1.02 mmol) in CH₃CN (10 mL) was refluxed for 36 h. It was cooled to RT, diluted with dichloromethae (50 mL) and washed with H₂O (10 mL), dried and concentrated. The residue was purified by chromatography on silica gel and converted to HCl salt to give the title compound. ¹HNMR (400 MHz, DMSO-d₆): δ 8.60 (s, 1H), 8.17 (m, 1H), 7.95 (m, 1H), 7.70 (d, 1H), 7.60-7.40 (m, 3H), 7.20 (d, 1H), 7.00 (d, 1H), 6.65 (m, 2H), 6.50 (m, 1H), 4.00 (t, 2H), 3.80-3.10 (m, 10H), 2.80 (s, 2H), 2.00-1.70 (m, 4H). MS: 473 (M⁺+1).

### Example 60

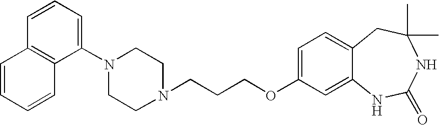

### 4,4-Dimethyl-8-[3-(4-naphthalen-1-yl-piperazin-1-yl)-propoxy]-1,3,4,5-tetrahydro-benzo[d][1,3]diazepin-2-one

A mixture of 8-(3-chloro-propoxy)-4,4-dimethyl-1,3,4,5-tetrahydro-benzo[d][1,3]diazepin-2-one (54), (0.2 g, 0.71 mmol), 1-naphthalen-1-yl-piperazine hydrochloride, ((US Pat App Pub No 20050043309, 18 mg, 0.84 mmol), NaI (0.21 g, 1.42 mmol) and K₂CO₃ (0.29 g, 1.02 mmol) in CH₃CN (10 mL) was refluxed for 36 h. It was cooled to RT, diluted with dichloromethane (50 mL) and washed with H₂O (10 mL), dried and concentrated. The residue was purified by chromatography on silica gel to give the title compound. ¹HNMR (400 MHz, CDCl₃): δ 8.20 (m, 1H), 8.17 (m, 1H), 7.85 (m, 1H), 7.70-7.40 (m, 5H), 7.10 (d, 1H), 6.96 (d, 1H), 6.80 (s, 1H), 6.60 (m, 1H), 5.00 (s, 1H), 4.10 (t, 2H), 3.20 (m, 4H), 3.00-2.60 (m, 8H), 2.10 (br s, 2H), 1.60 (br s, 2H), 1.20 (s, 6H). MS: 459 (M⁺+1).

### Example 61

### 8-Methoxy-1,3,4,5-tetrahydrobenzo[b]azepin-2-one (56)

To a solution of 7-methoxy-1-tetralone 55 (5.00 g, 28.4 mmol) in a 1:1 mixture of ethanol and water (70 mL) was added hydroxylamine hydrochloride (2.40 g, 34.1 mmol) and sodium acetate (4.70 g, 56.8 mmol) and the mixture heated to reflux. After 16 h, the reaction mixture was cooled to room temperature and a saturated sodium bicarbonate solution (50 mL) was added. The mixture was extracted with ethyl acetate (3×75 mL) and the organic layers were combined, washed with brine (75 mL), dried over sodium sulfate, filtered and concentrated to afford the intermediate oxime (5.4 g), which was directly taken for rearrangement without further purification. The oxime was added to a preheated solution of polyphosphoric acid (60 mL) at 115° C. and stirred for 5 min. The hot solution was poured into an ice/water mixture and stirred vigorously for 30 min. The precipitated solids were filtered, washed with water (1 L) and dried in a vacuum oven to afford the title compound (56) as an off-white solid: ¹H NMR (CDCl₃) δ 7.14 (s, 1H), 7.11 (s, 1H), 6.69 (dd, J=8.4, 2.6 Hz, 1H), 6.50 (d, J=2.5 Hz, 1H), 3.80 (s, 3H), 2.74 (t, J=7.2 Hz, 2H), 2.35 (t, J=7.3 Hz, 2H), 2.22-2.18 (m, 2H).

### 8-Hydroxy-1,3,4,5-tetrahydrobenzo[b]azepin-2-one (57)

A solution of 8-Methoxy-1,3,4,5-tetrahydro-benzo[b]azepin-2-one (56) (1.1 g, 5.8 mmol) in dichloromethane (60 mL) was cooled to −78° C., and a 1.0 M solution of boron tribromide (12.6 mL, 12.6 mmol) in dichloromethane was added dropwise. The reaction mixture was allowed to warm to room temperature and stir overnight. After 16 h, the reaction mixture was poured into ice/water (30 mL) mixture and stirred vigorously to evaporate the dichloromethane. The solids obtained were filtered, washed with water, and dried to afford the title compound (57) as a tan solid: ¹H NMR (CD₃OD) δ 7.03 (d, J=8.2 Hz, 1H), 6.57 (dd, J=8.2, 2.5 Hz, 1H), 6.47 (d, J=2.4 Hz, 1H), 2.66 (t, J=7.0 Hz, 2H), 2.26 (t, J=6.9 Hz, 2H), 2.16 (t, J=7.0 Hz, 2H); MS (ESI) m/z 178 [C₁₀H₁₁NO₂+H]⁺.

### 8-(4-Chlorobutoxy)-1,3,4,5-tetrahydro-benzo[b]azepin-2-one (58)

Following the procedure used for the synthesis above, 8-Hydroxy-1,3,4,5-tetrahydro-benzo[b]azepin-2-one (57) (0.40 g, 2.3 mmol), 1-bromo-4-chlorobutane (0.77 g, 4.5 mmol), and cesium carbonate (1.10 g, 3.38 mmol) in ethanol (5 mL) afforded the title compound (58) as a white solid: ¹H NMR (CDCl₃) δ 7.72 (br s, 1H), 7.11 (d, J=8.2 Hz, 1H), 6.67 (dd, J=8.3, 2.5 Hz, 1H), 6.52 (d, J=2.5 Hz, 1H), 3.97 (t, J=5.5 Hz, 2H), 3.62 (t, J=6.1 Hz, 2H), 2.73 (t, J=7.2 Hz, 2H), 2.36 (t, J=7.4 Hz, 2H), 2.24-2.17 (m, 2H), 2.08-1.94 (m, 4H); MS (ESI) m/z 268 [C₁₄H₁₈ClNO₂+H]⁺.

### Example 62

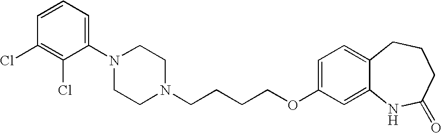

### 8-{4-[4-(2,3-Dichlorophenyl)piperazin-1-yl]butoxy}-1,3,4,5-tetrahydro-benzo[b]azepin-2-one

Following the general procedure of Example 52, 8-(4-Chloro-butoxy)-1,3,4,5-tetrahydro-benzo[b]azepin-2-one (58) (0.32 g, 1.2 mmol), 2,3-dichlorophenyl piperazine hydrochloride (0.39 g, 1.4 mmol), sodium iodide (0.21 g, 1.4 mmol) and potassium carbonate (0.50 g, 3.6 mmol) in acetonitrile (20 mL) afforded the title compound as a white solid: ¹H NMR (CDCl₃) δ 7.16-7.09 (m, 4H), 6.96 (d, J=3.4 Hz, 1H), 6.68 (d, J=8.3 Hz, 1H), 6.50 (d, J=2.4 Hz, 1H), 3.97 (t, J=6.2 Hz, 2H), 3.08 (br s, 4H), 2.73 (t, J=7.2 Hz, 2H), 2.67 (br s, 4H), 2.50 (t, J=7.3 Hz, 2H), 2.35 (t, J=7.3 Hz, 2H), 2.21-2.17 (m, 2H), 1.86-1.69 (m, 4H); MS (ESI) m/z 462 [C₂₄H₂₉Cl₂N₃O₂+H]⁺.

### Example 63

### Trifluoromethanesulfonic acid 2-oxo-2,3,4,5-tetrahydro-1H-benzo[d][1,3]-diazepin-8-yl ester (59)

To a stirred suspension of 8-hydroxy-1,3,4,5-tetrahydrobenzo[d][1,3]diazepin-2-one (29) (0.59 g, 3.3 mmol) in dry acetonitrile (20 mL) at 0° C. was added N,N-diisopropylethylamine (0.75 mL, 4.3 mmol) and N-phenyltrifluoromethanesulfonimide (1.54 g, 4.30 mmol). The reaction mixture was stirred at room temperature for 12 h and quenched with water (50 mL) and a 2 M hydrochloric acid solution (50 mL). The aqueous layer was extracted with ethyl acetate (3×100 mL) and the combined organic layers were washed with water (50 mL) and brine (50 mL), dried over sodium sulfate, filtered and concentrated in vacuo. The crude material was purified by silica gel chromatography (eluant:ethyl acetate) to afford the title compound (59) as a pale yellow solid. ¹H NMR (CDCl₃) δ 8.86 (s, 1H), 7.08 (d, J=8.4 Hz, 1H), 6.91 (d, J=2.5 Hz, 1H), 6.79 (dd, J=8.4, 2.5 Hz, 1H), 6.26 (br s, 1H), 3.48-3.44 (m, 2H), 3.09-3.05 (m, 2H); MS (ESI) m/z 311 [C₁₀H₉F₃N₂O₄S+H]⁺.

### 8-(5-Chloropent-1-enyl)-1,3,4,5-tetrahydrobenzo[d][1,3]diazepin-2-one (60)

To a stirred solution of trifluoromethanesulfonic acid 2-oxo-2,3,4,5-tetrahydro-1H-benzo[d][1,3]-diazepin-8-yl ester (59) (0.72 g, 2.3 mmol) in dimethoxyethane (12 mL) was added tetrakis(triphenylphosphine)palladium(0) (130 mg, 0.12 mmol). The reaction vessel was evacuated and backfilled with nitrogen. A solution of (E)-5-chloro-1-pentaneboronic acid (0.72 g, 4.9 mmol) in dimethoxyethane (4 mL) was added to the reaction mixture followed by a solution of sodium carbonate (0.52 g, 4.9 mmol) in water (3 mL). The reaction mixture was heated to reflux for 4 h, cooled to room temperature and diluted with ethyl acetate (100 mL). The organic layer was washed with water (20 mL) and brine (20 mL), dried over sodium sulfate, filtered and concentrated in vacuo. The crude material was purified by silica gel chromatography (eluant: 95:5 dichloromethane/methanol) to afford the title compound (60) as a white solid. ¹H NMR (CDCl₃) δ 9.20 (br s, 1H), 7.43 (br s, 1H), 7.04 (s, 1H), 6.91 (d, J=7.9 Hz, 1H), 6.84 (d, J=7.8 Hz, 1H), 6.26 (d, J=15.9 Hz, 1H), 6.11 (dt, J=15.8, 6.8 Hz, 1H), 3.47 (t, J=6.6 Hz, 2H), 3.38-3.36 (m, 2H), 2.97 (t, J=4.5 Hz, 2H), 2.23 (q, J=7.0 Hz, 2H), 1.83 (quintet, J=6.7 Hz, 2H); ¹³C NMR (CDCl₃) δ 158.8, 137.9, 136.8, 130.2, 130.1, 128.8, 127.7, 119.8, 116.6, 44.4, 42.5, 34.8, 31.9, 30.0; MS (ESI) m/z 265 [C₁₄H₁₇ClN₂O+H]⁺.

### Example 64

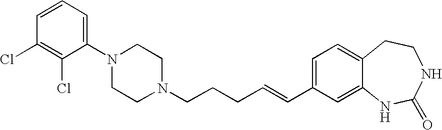

### 8-{5-[4-(2,3-Dichlorophenyl)piperazin-1-yl]pentyl-1-enyl}-1,3,4,5-tetrahydrobenzo[d]1,3]diazepin-2-one

To a stirred solution of 8-(5-chloropent-1-enyl)-1,3,4,5-tetrahydrobenzo[d][1,3]-diazepin-2-one (60) (0.52 g, 2.0 mmol) in acetonitrile (30 mL) was added 3,2-dichlorophenyl piperazine hydrochloride (0.63 g, 2.36 mmol), sodium iodide (0.44 g, 3.0 mmol) and potassium carbonate (0.82 g, 5.9 mmol). The reaction mixture was heated to reflux for 48 h, cooled to room temperature and diluted with water. The aqueous phase was extracted with ethyl acetate (3×50 mL) and the combined organic layers were dried over sodium sulfate. Solvent removal in vacuo followed by purification of the residue by silica gel chromatography (eluant: 95:5 dichloromethane/methanol) afforded the title compound as a white solid. mp 219-220° C.; ¹H NMR (CDCl₃) □ 7.17-7.14 (m, 2H), 7.04 (br s, 1H), 6.98-6.91 (m, 3H), 6.76-6.75 (m, 1H), 6.33 (d, J=15.9, 1H), 6.19 (dt, J=15.8, 6.6 Hz, 1H), 5.59 (br s, 1H), 3.46-3.41 (m, 2H), 3.09-3.08 (m, 4H), 3.05-3.01 (m, 2H), 2.67-2.66 (m, 4H), 2.48 (t, J=7.5 Hz, 2H), 2.25 (q, J=6.9 Hz, 2H), 1.72 (quintet, J=7.6 Hz, 2H); MS (ESI) m/z 459 [C₂₄H₂₈Cl₂N₄O+H]⁺.

### Example 65

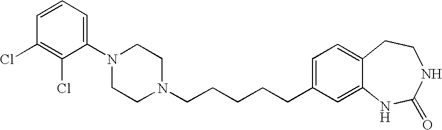

### 8-{5-[4-(2,3-Dichlorophenyl)piperazin-1-yl]pentyl}-1,3,4,5-tetrahydrobenzo[d]1,3]diazepin-2-one

To a Parr bottle containing platinum (IV) oxide (55 mg) was added methanol (60 mL) under an atmosphere of nitrogen. The mixture was shaken with hydrogen (40 psi) for 5 min to pre-reduce the catalyst. A solution of 8-{5-[4-(2,3-dichlorophenyl)piperazin-1-yl]pentyl-1-enyl}-1,3,4,5-tetrahydrobenzo[d]1,3]-diazepin-2-one (0.55 g, 1.2 mmol) in methanol (100 mL) was added to the pre-reduced catalyst and the reaction mixture shaken for 2 h under hydrogen (50 psi) atmosphere. The mixture was filtered through a pad of diatomaceous earth, concentrated and the residue was purified by silica gel chromatography (eluant: 95:5 ethyl acetate/methanol) to afford the title compound as a white solid. mp 159-161° C. (re-crystallized from methanol); ¹H NMR (CDCl₃) δ 7.16-7.14 (m, 3H), 7.00-6.95 (m, 2H), 6.76 (dd, J=7.7, 1.4 Hz, 1H), 6.63-6.60 (m, 1H), 5.72 (br s, 1H), 3.46-3.42 (m, 2H), 3.09-3.08 (m, 4H), 3.04-3.00 (m, 2H), 2.66-2.64 (m, 4H), 2.55 (t, J=7.5 Hz, 2H), 2.41 (t, J=7.5 Hz, 2H), 1.67-1.51 (m, 4H), 1.41-1.36 (m, 2H); MS (ESI) m/z 461 [C₂₄H₃₀Cl₂N₄O+H]⁺.

### Example 66

### 2-Methyl-2-(2-nitrophenyl)propionitrile (62)

A solution of (2-nitro-phenyl)-acetonitrile (61) (20.2 g, 125 mmol) and iodomethane (17.0 mL, 274 mmol) in tetrahydrofuran (170 mL) was added dropwise via an addition funnel to a slurry of sodium hydride (12.5 g, 60% in mineral oil, 310 mmol) in tetrahydrofuran (300 mL) at 0° C. under nitrogen. After the addition was complete, the cooling bath was removed, and the mixture stirred at room temperature overnight. The mixture was quenched with a saturated ammonium chloride solution (500 mL) and the layers were separated. The aqueous layer was extracted with ethyl acetate (3×200 mL). The combined organic extracts were washed with brine (250 mL), dried over sodium sulfate, filtered and concentrated in vacuo to provide the title compound (62) (product contained residual mineral oil) as a red oil. ¹H NMR (CDCl₃) δ 7.70-7.61 (m, 3H), 7.52-7.46 (m, 1H), 1.90 (s, 6H); MS (ESI) m/z 191 [C₁₀H₁₀N₂O₂+H]⁺.

### 2-Methyl-2-(2-nitrophenyl)propylamine (63)

A solution of 2-methyl-2-(2-nitrophenyl)propionitrile 62 (4.10 g, 21.6 mol) in anhydrous tetrahydrofuran (80 mL) under nitrogen was cooled to 0° C. with vigorous stirring. Borane-tetrahydrofuran complex (43.0 mL, 43.0 mmol, 1 M solution in tetrahydrofuran) was added dropwise via syringe over 15 min. Once the addition was complete, the mixture was heated to reflux for 4 h, then allowed to cool to room temperature. Methanol was added in small portions (ca. 10 mL) until gas evolution ceased. A 3 M hydrochloric acid solution (200 mL) was added, and the mixture was heated to reflux for 2 h, then allowed to cool. The volatile solvents were removed in vacuo, and the remaining aqueous layer was diluted with water (500 mL). The aqueous solution was made strongly alkaline (pH >10) by the addition of a 2 M sodium hydroxide solution, then extracted with ethyl acetate (3×200 mL). The combined organic extracts were dried over sodium sulfate, filtered and concentrated in vacuo to provide the title compound (63) as an orange oil. ¹H NMR (CDCl₃) δ 7.53-7.44 (m, 2H), 7.34-7.31 (m, 2H), 2.94 (s, 2H), 1.37 (s, 6H); MS (ESI) m/z 195 [C₁₀H₁₄N₂O₂+H]⁺.

### [2-(4-Bromo-2-nitro-phenyl)-2-methyl-propyl]-carbamic acid tert-butyl ester (64)

To a stirred solution of 2-methyl-2-(2-nitrophenyl)propylamine 63 (2.45 g, 12.6 mmol) in trifluoroacetic acid (10 mL) and concentrated sulfuric acid (4 mL) was added portionwise N-bromosuccinimide (4.50 g, 25.2 mmol). The reaction mixture was stirred at room temperature for 48 h and the volatiles were removed in vacuo. The residue was carefully poured into ice/water and the aqueous phase was made alkaline (pH 10) by the addition of a 6 M sodium hydroxide solution. A solution of di-tert-butyldicarbonate (5.50 g, 25.3 mmol) in 1,4-dioxane (60 mL) was added to the alkaline mixture and stirred at room temperature overnight. Acidification to pH 3 using a 2 M hydrochloric acid solution, was followed by extraction with ethyl acetate (3×150 mL). The combined organic layers were washed with brine (100 mL), dried over sodium sulfate and filtered. Solvent removal in vacuo followed by purification of the residue by silica gel chromatography (eluant: 80:20 hexanes/ethyl acetate) afforded [2-(4-bromo-2-nitro-phenyl)-2-methyl-propyl]-carbamic acid tert-butyl ester (64) as a colorless oil: MS (ESI) m/z 372 [C₁₅H₂₁BrN₂O₄+H]⁺.

### [2-(2-Amino-4-bromophenyl)-2-methylpropyl]carbamic Acid tert-Butyl Ester (65)

To a stirred solution of the [2-(4-bromo-2-nitro-phenyl)-2-methyl-propyl]-carbamic acid tert-butyl ester (64) (1.0 g, 2.7 mmol), obtained as described above, in ethanol (25 mL) and glacial acetic acid (25 mL) was added iron powder (0.89 g, 16 mmol). The reaction mixture was heated to reflux for 45 min, then cooled to room temperature. Ethyl acetate (100 mL), water (50 mL) and sodium carbonate (13.5 g) were added, and the mixture was stirred for 45 min. The solids were removed by filtration through diatomaceous earth, and the filter cake was rinsed with ethyl acetate (4×50 mL). The filtrate layers were separated, and the aqueous layer was extracted with ethyl acetate (3×50 mL). The organic layers were combined, dried over sodium sulfate, filtered and concentrated in vacuo. Purification of the residue using silica gel chromatography (eluant: 95:5 hexanes/ethyl acetate) afforded the title compound (65) as a colorless oil. ¹H NMR (CDCl₃) δ 6.99 (d, J=8.3 Hz, 1H), 6.81 (dd, J=8.4, 2.0 Hz, 1H), 6.78 (d, J=2.0 Hz, 1H), 4.66-4.65 (m, 1H), 4.23 (br s, 2H), 3.39 (d, J=6.6 Hz, 2H), 1.42 (s, 9H), 1.33 (s, 6H); MS (ESI) m/z 343 [C₁₅H₂₃BrN₂O₂+H]⁺.

### 8-Bromo-5,5-dimethyl-1,3,4,5-tetrahydrobenzo[d][1,3]diazepin-2-one (66)

To a stirred solution of [2-(2-aminobromophenyl)-2-methylpropyl]carbamic acid tert-butyl ester (65) (0.50 g, 1.5 mmol) in 1,4-dioxane (10 mL) was added a 2 M hydrogen chloride solution in ether (20 mL). The reaction mixture was stirred at room temperature overnight. Solvent removal in vacuo afforded crude diamine (0.7 g, >99%) as a white solid, MS (ESI) m/z 243 [C₁₀H₁₅BrN₂+H]⁺.

To a stirred solution of the crude diamine (0.70 g, 2.2 mmol) in dry tetrahydrofuran (40 mL) was added triethylamine (1.00 mL, 6.60 mmol) and solid 1,1′-carbonyldiimidazole (0.54 g, 3.3 mmol) in small portions over 5 min. The reaction mixture was heated to reflux for 16 h, cooled to room temperature and was partitioned between ethyl acetate (100 mL) and a 1 M hydrochloric acid solution (50 mL). The organic phase was removed and the aqueous layer extracted with ethyl acetate (3×100 mL). The combined organic extracts were washed with brine (100 mL), dried over sodium sulfate and filtered. Solvent removal in vacuo followed by purification of the residue by silica gel chromatography (eluant: 20:80 hexanes/ethyl acetate) afforded the title compound (66) as a pale yellow solid. ¹H NMR (CDCl₃) δ 7.98 (s, 1H), 7.15-7.13 (m, 1H), 7.05-7.02 (m, 2H), 6.26 (br s, 1H), 3.20 (d, J=5.1 Hz, 2H), 1.33 (s, 6H); MS (ESI) m/z 269 [C₁₁H₁₃BrN₂O+H]⁺.

### 8-(5-Chloro-pent-1-enyl)-5,5-dimethyl-1,3,4,5-tetrahydrobenzo[d][1,3]-diazepin-2-one (67)

To a stirred solution of 8-bromo-5,5-dimethyl-1,3,4,5-tetrahydrobenzo[d][1,3]-diazepin-2-one (66) (0.15 g, 0.57 mmol) in dimethoxyethane (6 mL) was added tetrakis(triphenylphosphine)palladium (0) (33 mg, 0.030 mmol). The reaction vessel was evacuated and backfilled with nitrogen. A solution of (E)-5-Chloro-1-penteneboronic acid (0.18 g, 1.2 mmol) in dimethoxyethane (2 mL) was added to the reaction mixture followed by a solution of sodium carbonate (0.13 g, 1.20 mmol) in water (1 mL). The reaction mixture was heated to reflux for 2 h, cooled to room temperature and diluted with ethyl acetate (100 mL). The organic layer was washed with water (20 mL) and brine (20 mL), dried over sodium sulfate, filtered and concentrated in vacuo. The crude material was purified by silica gel chromatography (eluant: 95:5 dichloromethane/methanol) to afford the intermediate chloride (67) (0.17 g, 98%) as a white solid: MS (ESI) m/z 293 [C₁₆H₂₁ClN₂O+H]⁺.

To a stirred solution of the above chloride (0.17 g, 0.57 mmol) in acetonitrile (16 mL) was added dichlorophenyl piperazine hydrochloride (0.22 g, 0.80 mmol), sodium iodide (0.17 g, 1.2 mmol) and potassium carbonate (0.24 g, 1.7 mmol). The reaction mixture was heated to reflux for 48 h, cooled to room temperature and diluted with water. The aqueous phase was extracted with ethyl acetate (3×50 mL) and the combined organic layers were dried over sodium sulfate. Solvent removal in vacuo followed by purification of the residue by silica gel chromatography (eluant: 95:5 dichloromethane/methanol) afforded the title compound as a white solid. ¹H NMR (CDCl₃) δ 8.35 (s, 1H), 7.20 (d, J=8.6 Hz, 1H), 7.15-7.11 (m, 2H), 7.10-7.09 (m, 1H), 6.96-6.93 (m, 1H), 6.92 (s, 2H), 6.28 (d, J=15.8 Hz, 1H), 6.18 (dt, J=15.8, 6.4 Hz, 1H), 3.16 (d, J=4.8 Hz, 2H), 3.05 (br s, 4H), 2.61 (br s, 4H), 2.41 (t, J=7.3 Hz, 2H), 2.18 (q, J=6.9 Hz, 2H), 1.66 (quintet, J=7.5 Hz, 2H), 1.30 (s, 6H); MS (ESI) m/z 487 [C₂₆H₃₂Cl₂N₄O+H]⁺.

### Example 67

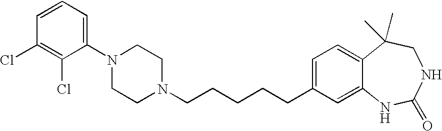

### 8-{5-[4-(2,3-Dichlorophenyl)piperazin-1-yl]pentyl}-5,5-dimethyl-1,3,4,5-tetrahydro-benzo[d][1,3]diazepin-2-one Methanesulfonic Acid

To a Parr bottle containing platinum (IV) oxide (17 mg) was added methanol (20 mL) under an atmosphere of nitrogen. The mixture was shaken with hydrogen (40 psi) for 5 min to pre-reduce the catalyst. A solution of 8-{5-[4-(2,3-dichlorophenyl)piperazin-1-yl]pent-1-enyl}-5,5-dimethyl-1,3,4,5-tetrahydrobenzo[d][1,3]diazepin-2-one (0.17 g, 0.34 mmol) in methanol (100 mL) was added to the pre-reduced catalyst and the reaction mixture was shaken for 2 h under hydrogen (50 psi). The mixture was filtered through a pad of diatomaceous earth, concentrated and the residue was purified by silica gel chromatography (eluant: 95:5 ethyl acetate/methanol) to afford the hydrogenated product (0.15 g, 89%) as a colorless oil. The oil (0.15 g, 0.31 mmol) was dissolved in EtOAc (5 mL) and treated with methanesulfonic acid (2 M in ether, 0.16 mL, 0.32 mmol). After stirring for 10 min the resulting precipitate was collected by filtration, washed with ether (4×10 mL), and dried in a vacuum oven at 55° C. overnight to give the title compound as a white solid. mp 215-218° C. (re-crystallized from acetonitrile); ¹H NMR (DMSO-d₆) δ 9.39 (br s, 1H), 8.21 (d, J=1.7 Hz, 1H), 7.42-7.34 (m, 2H), 7.24-7.20 (m, 2H), 7.15-7.14 (m, 1H), 6.84 (d, J=1.3 Hz, 1H), 6.70 (dd, J=8.1, 1.3 Hz, 1H), 3.62-3.58 (m, 2H), 3.48-3.44 (m, 2H), 3.19-3.16 (m, 4H), 3.01-2.99 (m, 4H), 2.46-2.44 (m, 2H), 2.30 (s, 3H), 1.64-1.52 (m, 4H), 1.34-1.29 (m, 2H), 1.21 (s, 6H); MS (ESI) m/z 489 [C₂₆H₃₄Cl₂N₄O+H]⁺.

### Example 68

### 8-(5-Chloropent-1-enyl)-5,5-dimethyl-1,3,4,5-tetrahydrobenzo[b]azepin 2-one (68)

To a solution of 8-bromo-5,5-dimethyl-1,3,4,5-tetrahydrobenzo[b]azepin-2-one (34) (1.00 g, 3.72 mmol) in ethylene glycol dimethyl ether (20 mL) under argon was added tetrakis(triphenylphosphine)palladium(0) (0.13 g, 0.11 mmol). A slurry (E)-5-chloro-1-penteneboronic acid (1.24 g, 8.40 mmol) in ethylene glycol dimethyl ether (4 mL) was added to the reaction mixture followed by a 2 M solution of aqueous sodium carbonate (0.84 g in 4 mL) and the mixture was heated to reflux. After 17 h, the reaction mixture was cooled and concentrated in vacuo. The residue was diluted with tetrahydrofuran (100 mL), stirred for 15 min and the solids filtered. The filtrate was concentrated and the residue purified by silica gel column chromatography (eluant: 75:25 hexanes/ethyl acetate) to afford the title compound (68) as a gummy liquid. ¹H NMR (CDCl₃) δ 7.48 (s, 1H), 7.34 (d, J=8.2 Hz, 1H), 7.13 (dd, J=8.2, 1.4 Hz, 1H), 6.89 (d, J=1.2 Hz, 1H), 6.39 (d, J=15.9 Hz, 1H), 6.36-6.10 (m, 1H), 3.58 (t, J=6.5 Hz, 2H), 2.38 (q, J=6.3 Hz, 4H), 2.11-1.92 (m, 4H), 1.38 (s, 6H); MS (ESI) m/z 292 [C₁₇H₂₂ClNO+H]⁺.

### Example 69

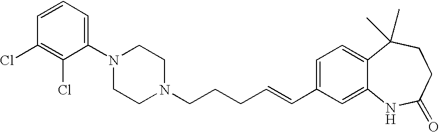

### 8-{5-[4-2,3-Dichlorophenyl)-piperazin-1-yl]pent-1-enyl}-5,5-dimethyl-1,3,4,5-tetrahydrobenzo[b]azepin-2-one

A procedure similar to the one described in Example 64 was used to produce 8-{5-[4-(2,3-Dichlorophenyl)-piperazin-1-yl]pent-1-enyl}-5,5-dimethyl-1,3,4,5-tetrahydrobenzo[b]azepin-2-one, as follows. To a stirred solution of 8-(5-chloropent-1-enyl)-5,5-dimethyl-1,3,4,5-tetrahydrobenzo[b]azepin-2-one (68) (1.00 g, 3.43 mmol) in acetonitrile (70 mL) was added 2,3-dichlorophenyl piperazine hydrochloride (1.1 g, 4.1 mmol), sodium iodide (0.62 g, 4.1 mmol), and potassium carbonate (1.42 g, 10.3 mmol). The reaction mixture was heated to reflux for 48 h, cooled to room temperature and diluted with water. The aqueous phase was extracted with ethyl acetate, and the combined organic layers were dried over sodium sulfate. Solvent removal in vacuo followed by purification of the residue by silica gel afforded the title compound as a white solid. mp 108-109° C.; ¹H NMR (CDCl₃) δ 7.34 (d, J=8.2 Hz, 1H), 7.18 (s, 1H), 7.22-7.12 (m, 3H), 6.98 (dd, J=8.0, 3.6 Hz, 1H), 6.86 (d, J=1.8 Hz, 1H), 6.36 (d, J=15.8 Hz, 1H), 6.23 (dt, J=15.8, 6.5 Hz, 1H), 3.08 (br s, 4H), 2.65 (br s, 4H), 2.47 (t, J=7.6 Hz, 2H), 2.39 (t, J=6.8 Hz, 2H), 2.26 (q, J=7.0 Hz, 2H), 2.08 (t, J=6.7 Hz, 2H), 1.71 (q, J=7.6 Hz, 2H), 1.26 (s, 6H); MS (ESI) m/z 486 [C₂₇H₃₃Cl₂N₃O+H]⁺.

### Example 70

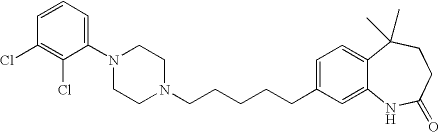

### 8-{5-[4-(2,3-Dichlorophenyl)piperazin-1-yl]pentyl}-5,5-dimethyl-1,3,4,5-tetrahydrobenzo[b]azepin-2-one Methanesulfonic Acid

To a suspension of platinum (IV) oxide (60 mg) in methanol (70 mL) was added 8-{5-[4-(2,3-dichlorophenyl)-piperazin-1-yl]pent-1-enyl}-5,5-dimethyl-1,3,4,5-tetrahydrobenzo[b]azepin-2-one (0.20 g, 0.41 mmol) and the mixture was shaken on a Parr hydrogenator under hydrogen (50 psi) atmosphere for 90 min. The reaction mixture was filtered through diatomaceous earth and the filtrate concentrated. The residue (0.18 g) was dissolved in ethyl acetate (3 mL) and a solution of methanesulfonic acid (0.19 mL, 2.0 M solution in ether) was added and stirred for 15 min. The precipitated solids were filtered and dried in a vacuum oven to afford the title compound as a white solid. mp 168-170° C.; ¹H NMR (CDCl₃) δ 11.2 (s, 1H), 7.70 (s, 1H), 7.30 (d, J=8.1 Hz, 1H), 7.22-7.10 (m, 3H), 7.04 (dd, J=7.8, 1.6 Hz, 1H), 6.94 (dd, J=8.1, 1.5 Hz, 1H), 6.77 (d, J=1.4 Hz, 1H), 3.69 (d, J=11.1 Hz, 2H), 3.55-3.37 (m, 4H), 3.14-3.00 (m, 4H), 2.83 (s, 3H), 2.61 (t, J=7.4 Hz, 2H), 2.38 (t, J=7.0 Hz, 2H), 2.08 (t, J=7.0 Hz, 2H), 1.98-1.90 (m, 2H), 1.81-1.60 (m, 2H), 1.52-1.30 (m, 7H); MS (ESI) m/z 488 [C₂₇H₃₅Cl₂N₃O+H]⁺.

### Example 71

### Dopamine D₂ Receptor Binding Assay

The compounds produced as described in Examples 1-70, above were each tested in a Dopamine D₂ Receptor Binding Assay, as follows.

[³H]Spiperone binding to a membrane preparation from CHO-hD₂L cells was carried out in 250 μl of 50 mM Tris-HCl buffer containing 100 mM NaCl, 1 mM MgCl₂ and 1% DMSO at pH 7.4. Duplicate samples containing (in order of addition) the test compounds, 0.4 nM [³H]spiperone and approximately 12 μg protein were incubated for 120 minutes at room temperature. Bound radioligand was separated by rapid filtration under reduced pressure through Whatman GF/B glass fiber filters previously treated with 0.3% polyethyleneimine. Radioactivity retained on the filter is determined by liquid scintillation spectrophotometry. Specific binding was determined in the presence of 1 mM haloperidol was 95%. Results are provided in Table 1 below.

TABLE 1
Dopamine D₂ Receptor Binding

| EX- | | |
|---|---|---|
| AM- | | D₂ Ki |
| PLE | COMPOUND | (nM) |
| 2 | 2-[4-(4-Naphthalen-1-yl-piperazin-1-yl)-butoxy]-5,6,7,9- | 3.87 |
| | tetrahydro-pyrido[2,3-b]azepin-8-one | |
| 3 | 2-{4-[4-(2,3-dichloro-phenyl)-piperazin-1-yl]-butoxy}- | 5.48 |
| | 5,6,7,9-tetrahydro-pyrido[2,3-b]azepin-8-one | |
| 4 | 2-[4-(4-Chroman-8-yl-piperazin-1-yl)-butoxy]-5,6,7,9- | 2 |
| | tetrahydro-pyrido[2,3-b]azepin-8-one | |
| 5 | 2-{4-[4-(5,6,7,8-Tetrahydro-naphthalen-1-yl)-piperazin-1- | 2.45 |
| | yl]-butoxy}-5,6,7,9-tetrahydro-pyrido[2,3-b]azepin-8-one | |
| 6 | 2-[4-(4-Indan-4-yl-piperazin-1-yl)-butoxy]-5,6,7,9- | 1 |
| | tetrahydro-pyrido[2,3-b]azepin-8-one | |
| 7 | 2-{4-[4-(2,3-Dihydro-benzofuran-7-yl)-piperazin-1-yl]- | 3.46 |
| | butoxy}-5,6,7,9-tetrahydro-pyrido[2,3-b]azepin-8-one | |
| 8 | 2-{4-[4-(7-Fluoro-naphthalen-1-yl)-piperazin-1-yl]- | 1 |
| | butoxy}-5,6,7,9-tetrahydro-pyrido[2,3-b]azepin-8-one | |
| 9 | 2-{4-[4-(3,4-Dihydro-2H-benzo[b][1,4]dioxepin-6-yl)- | 2 |
| | piperazin-1-yl]-butoxy}-5,6,7,9-tetrahydro-pyrido[2,3- | |
| | b]azepin-8-one | |
| 10 | 8-{4-[4-(8-Oxo-6,7,8,9-tetrahydro-5H-pyrido[2,3- | 2.79 |
| | b]azepin-2-yloxy)-butyl]-piperazin-1-yl}-naphthalene-2- | |
| | carbonitrile | |
| 11 | 2-{4-[4-(1-Methyl-2-oxo-1,2,3,4-tetrahydro-quinolin-8- | 18.9 |
| | yl)-piperazin-1-yl]-butoxy}-5,6,7,9-tetrahydro-pyrido[2,3- | |
| | b]azepin-8-one | |
| 13 | 2-[4-(4-Indan-4-yl-piperazin-1-yl)-butoxy]-5,6,7,9- | 0.59 |
| | tetrahydro-1,7,9-triaza-benzocyclohepten-8-one | |
| 14 | 2-{4-[4-(2,3-Dichloro-phenyl)-piperazin-1-yl]-butoxy}- | 1 |
| | 5,6,7,9-tetrahydro-1,7,9-triaza-benzocyclohepten-8-one | |
| 15 | 2-[4-(4-Naphthalen-1-yl-piperazin-1-yl)-butoxy]-5,6,7,9- | 1 |
| | tetrahydro-1,7,9-triaza-benzocyclohepten-8-one | |
| 16 | 2-{4-[4-(5,6,7,8-Tetrahydro-naphthalen-1-yl)-piperazin-1- | 1.41 |
| | yl]-butoxy}-5,6,7,9-tetrahydro-1,7,9-triaza- | |
| | benzocyclohepten-8-one | |
| 17 | 2-[4-(4-Chroman-8-yl-piperazin-1-yl)-butoxy]-5,6,7,9- | 1 |
| | tetrahydro-1,7,9-triaza-benzocyclohepten-8-one | |
| 18 | 2-{4-[4-(7-Fluoro-naphthalen-1-yl)-piperazin-1-yl]- | 1.79 |
| | butoxy}-5,6,7,9-tetrahydro-1,7,9-triaza- | |
| | benzocyclohepten-8-one | |
| 19 | 2-{4-[4-(2,3-Dihydro-benzofuran-7-yl)-piperazin-1-yl]- | 0.4 |
| | butoxy}-5,6,7,9-tetrahydro-1,7,9-triaza- | |
| | benzocyclohepten-8-one | |
| 20 | 2-{4-[4-(3,4-Dihydro-2H-benzo[b][1,4]dioxepin-6-yl)- | 1 |
| | piperazin-1-yl]-butoxy}-5,6,7,9-tetrahydro-1,7,9-triaza- | |
| | benzocyclohepten-8-one | |
| 21 | 2-{4-[4-(7-Methoxy-naphthalen-1-yl)-piperazin-1-yl]- | 0.4 |
| | butoxy}-5,6,7,9-tetrahydro-1,7,9-triaza- | |
| | benzocyclohepten-8-one | |
| 22 | 8-{4-[4-(8-Oxo-6,7,8,9-tetrahydro-5H-1,7,9-triaza- | 0.4 |
| | benzocyclohepten-2-yloxy)-butyl]-piperazin-1-yl}- | |
| | naphthalene-2-carbonitrile | |
| 23 | 2-{4-[4-(1-Methyl-2-oxo-1,2,3,4-tetrahydro-quinolin-8- | 3 |
| | yl)-piperazin-1-yl]-butoxy}-5,6,7,9-tetrahydro-1,7,9- | |
| | triaza-benzocyclohepten-8-one | |
| 25 | 8-{4-[4-(2,3-Dichlorophenyl)piperazin-1-yl]butoxy}- | 2.45 |
| | 3-methyl-1,3,4,5-tetrahydro-benzo[d][1,3]diazepin-2- | |
| | one | |
| 28 | 8-{3-[4-(2,3-Dichlorophenyl)piperazin-1-yl]propoxy}- | 4.47 |
| | 1,3,4,5-tetrahydrobenzo[d][1,3]diazepin-2-one | |
| 29 | 8-{4-[4-(2,3-Dichlorophenyl)piperazin-1-yl]butoxy}- | 4.47 |
| | 1,3,4,5-tetrahydrobenzo[d][1,3]diazepin-2-one | |
| 30 | 8-{4-[4-(2-Chloro-4-fluoro-3-methyl-phenyl)-piperazin-1- | 2 |
| | yl]-butoxy}-1,3,4,5-tetrahydro-benzo[d][1,3]diazepin-2- | |
| | one | |
| 31 | 8-{4-[4-(2-Chloro-4-fluoro-5-methyl-phenyl)-piperazin-1- | 6 |
| | yl]-butoxy}-1,3,4,5-tetrahydro-benzo[d][1,3]diazepin-2- | |
| | one | |
| 32 | 8-[4-(4-Naphthalen-1-yl-piperazin-1-yl)-butoxyl-1,3,4,5- | 3.45 |
| | tetrahydro-benzo[d][1,3]diazepin-2-one | |
| 33 | 8-{4-[4-(6-Ethyl-pyridin-2-yl)-piperazin-1-yl]-butoxy}- | 2.83 |
| | 1,3,4,5-tetrahydro-benzo[d][1,3]diazepin-2-one | |
| 34 | 8-{4-[4-(6-Isopropyl-pyridin-2-yl)-piperazin-1-yl]- | 2.68 |
| | butoxy}-1,3,4,5-tetrahydro-benzo[d][1,3]diazepin-2-one | |
| 35 | 8-{4-[4-(2-Chloro-4-fluoro-phenyl)-piperazin-1-yl]- | 3 |
| | butoxy}-1,3,4,5-tetrahydro-benzo[d][1,3]diazepin-2-one | |
| 36 | 8-{4-[4-(2,3-Dichloro-4-fluoro-phenyl)-piperazin-1-yl]- | 3 |
| | butoxy}-1,3,4,5-tetrahydro-benzo[d][1,3]diazepin-2-one | |
| 37 | 8-{4-[4-(6-Cyclopropyl-pyridin-2-yl)-piperazin-1-yl]- | 1.81 |
| | butoxy}-1,3,4,5-tetrahydro-benzo[d][1,3]diazepin-2-one | |
| 38 | 8-{4-[4-(7-Fluoro-naphthalen-1-yl)-piperazin-1-yl]- | 2.22 |
| | butoxy}-1,3,4,5-tetrahydro-benzo[d][1,3]diazepin-2-one | |
| 39 | 8-[4-(4-Benzo[1,2,5]thiadiazol-4-yl-piperazin-1-yl)- | 2.60 |
| | butoxy]-1,3,4,5-tetrahydro-benzo[d][1,3]diazepin-2-one | |
| 40 | 8-{4-[4-(5-Fluoro-naphthalen-1-yl)-piperazin-1-yl]- | 11.4 |
| | butoxy}-1,3,4,5-tetrahydro-benzo[d][1,3]diazepin-2-one | |
| 41 | 8-{3-[4-(2-Methoxy-quinolin-8-yl)-piperazin-1-yl]- | 1.04 |
| | propoxy}-1,3,4,5-tetrahydro-benzo[d][1,3]diazepin-2-one | |
| 42 | 8-{4-[4-(8-Fluoro-naphthalen-1-yl)-piperazin-1-yl]- | 2.32 |
| | butoxy}-1,3,4,5-tetrahydro-benzo[d][1,3]diazepin-2-one | |
| 43 | 8-[3-(4-Naphthalen-1-yl-piperazin-1-yl)-propoxy]-1,3,4,5- | 3.24 |
| | tetrahydro-benzo[d][1,3]diazepin-2-one | |
| 44 | 8-{3-[4-(7-Fluoro-naphthalen-1-yl)-piperazin-1-yl]- | 5.44 |
| | propoxy}-1,3,4,5-tetrahydro-benzo[d][1,3]diazepin-2-one | |
| 45 | 8-[4-(4-Isochroman-8-yl-piperazin-1-yl)-butoxy]-1,3,4,5- | 6.14 |
| | tetrahydro-benzo[d][1,3]diazepin-2-one | |
| 53 | 8-{3-[4-(2,3-Dichlorophenyl)piperazin-1-yl]- | 35 |
| | propoxy}-5,5-dimethyl-1,3,4,5- | |
| | tetrahydrobenzo[b]azepin-2-one | |
| 54 | 8-{4-[4-(2,3-Dichlorophenyl)piperazin-1-yl]-butoxy}- | 24 |
| | 5,5-dimethyl-1,3,4,5-tetrahydrobenzo[b]azepin-2-one | |
| 55 | 8-{5-[4-(2,3-Dichlorophenyl)piperazin-1-yl]- | 54.4 |
| | pentyloxy}-5,5-dimethyl-1,3,4,5- | |
| | tetrahydrobenzo[b]azepin-2-one | |
| 57 | 8-{4-[4-(2,3-Dichlorophenyl)piperazin-1-yl]butoxy}- | 12.5 |
| | 3,3-dimethyl-1,3,4,5-tetrahydrobenzo[b]azepin-2-one | |
| 59 | 4,4-Dimethyl-8-[4-(4-naphthalen-1-yl-piperazin-1-yl)- | 3 |
| | butoxy]-1,3,4,5-tetrahydro-benzo[d][1,3]diazepin-2-one | |
| 60 | 4,4-Dimethyl-8-[3-(4-naphthalen-1-yl-piperazin-1-yl)- | 9.38 |
| | propoxy]-1,3,4,5-tetrahydro-benzo[d][1,3]diazepin-2-one | |
| 62 | 8-{4-[4-(2,3-Dichloro-phenyl)-piperazin-1-yl]-butoxy}- | 6.48 |
| | 1,3,4,5-tetrahydro-benzo[b]azepin-2-one | |
| 64 | 8-{5-[4-(2,3-Dichloro-phenyl)-piperazin-1-yl]-pent-1- | 9.49 |
| | enyl}-1,3,4,5-tetrahydro-benzo[d][1,3]diazepin-2-one | |
| 65 | 8-{5-[4-(2,3-Dichloro-phenyl)-piperazin-1-yl]-pentyl}- | 4.47 |
| | 1,3,4,5-tetrahydro-benzo[d][1,3]diazepin-2-one | |
| 67 | 8-{5-[4-(2,3-Dichlorophenyl)piperazin-1-yl]pentyl}- | 25 |
| | 5,5-dimethyl-1,3,4,5-tetrahydro-benzo[d][1,3]diazepin- | |
| | 2-one | |
| 69 | 8-{5-[4-(2,3-Dichlorophenyl)-piperazin-1-yl]pent-1- | 70.9 |
| | enyl}-5,5-dimethyl-1,3,4,5-tetrahydrobenzo[b]azepin- | |
| | 2-one | |
| 70 | 8-{5-[4-(2,3-Dichlorophenyl)piperazin-1-yl]pentyl}- | 56.5 |
| | 5,5-dimethyl-1,3,4,5-tetrahydrobenzo[b]azepin-2-one | |

All of the compounds tested in this assay, as described above, exhibited Ki values less than 80 nM.

### Example 72

### Spontaneous Locomotor Activity Inhibition Test

Potential therapeutic effects of 2-{4-[4-(7-Fluoro-naphthalen-1-yl)-piperazin-1-yl]-butoxy}-5,6,7,9-tetrahydro-1,7,9-triaza-benzocyclohepten-8-one (hereinafter the “test compound”) were investigated in a spontaneous locomotor activity inhibition test in rats, a behavioral test predictive of antipsychotic activity. (A. E. Corbin, et. al.: PD 158771, a potential antipsychotic agent with D2/D3 partial agonist and 5-HT_{1A} agonist actions. II. Prelinical behavioral effects. Neuropharmacology 39: 1211-1221 (2000)) The testing was performed using 16-Beam Digiscan Animal Activity Monitors (Accuscan Electronics, Columbus, Ohio). Each test chamber consisted of a PLEXIGLAS® box measuring 16×16 inches. These boxes were housed inside stainless steel, ventilated sound-attenuating chambers (SACs). Lights inside the SACs were off. Male Sprague-Dawley rats (Harlan), weighing 180 to 200 g at the time of testing, were used. Rats were provided with food and water ad libitum, but were food deprived for 24 hours before testing. Rats were randomly assigned to vehicle or drug treatment groups, and were tested one per chamber. On the test day, rats were removed from the housing room and brought to the test room, where they were placed into PLEXIGLAS® isolator cages on corncob bedding, 4 rats per cage. Rats were dosed orally (PO) with the test compound or vehicle, and then returned to the isolators. After a 60-minute absorption period, each rat was placed into the test chamber, the chamber door closed, and recording of locomotor activity begun. Locomotor activity was recorded for 1 hour, in 5-minute blocks. The test compound was suspended in 1% cremophor EL, 1% HCl 1N, and 0.5% methocel in water. The test compound or the vehicle was administered in a volume of 5 mL/kg. Doses are expressed as the active moiety (ie, base). Locomotor activity was measured as centimeters traveled and expressed as percent inhibition from vehicle control. Statistical analysis was performed using a one-way ANOVA followed by a post hoc Dunnett's test. Data from the vehicle-treated control groups from each experiment were compared using a one-way ANOVA. Results are provided in Table 2 below.

TABLE 2
Effects of the Test Compound on Spontaneous Locomotor
Activity^{a}

| | Percent Decrease |
|---|---|
| Dose (mg/kg, PO) | from Control |
| 0.1 | 9.0 ± 37.9^{c} |
| 0.3 | 38.3 ± 47.2^{bd} |
| 1 | 53.7 ± 4.8^{bd} |
| 3 | 75.4 ± 3.5^{cd} |

^{a}Data are Mean percent decrease from control mean in 1 hour ± SEM.
^{b}N = 16.
^{c}N = 8.
^{d}p < 0.05 versus vehicle control; One-way ANOVA.

We claim:

1. A compound of formula 1 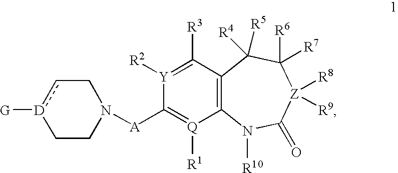
or a pharmaceutically acceptable salt thereof, wherein in formula 1:
R₁ is a group selected from formula (i) or formula (ii), below: 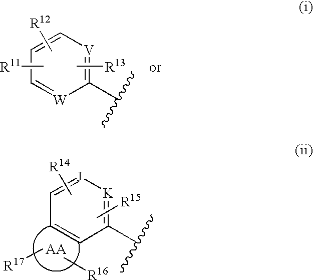
and wherein:
A is —(CH₂)ₘCH₂—, —(CH₂)ₘO—, or —(CH₂)ₘNH—, wherein m is an integer from 3 to 5, wherein two of the carbon atoms of —(CH₂)ₘCH₂— are optionally linked by a double bond, and wherein one or two of the carbon or nitrogen atoms of —(CH₂)ₘCH₂—, —(CH₂)ₘO—, and —(CH₂)ₘNH— can be substituted, optionally and independently, with a methyl or ethyl;
D is N, C, or CH, provided that when D is N, each carbon atom attached to D is attached through a single bond;
J and K are independently selected from N, CH, and C;
Q, Y, and Z are independently selected from N or C;
V and W are independently N, C, or CH;
ring AA is a saturated or unsaturated 5- 6- or 7-membered carbocyclic ring wherein one, two or three of the carbon atoms of ring AA that are not shared with the 6-membered aryl ring of group (ii) can be replaced, optionally and independently, by a nitrogen, oxygen or sulfur atom;
R¹, R² and R³ are independently selected from hydrogen, halo, cyano, hydroxy, (C₁-C₄) alkyl, and (C₁-C₄) alkoxy, wherein the alkyl moieties of the (C₁-C₄) alkyl or (C₁-C₄) alkoxy are straight or branched and can be optionally substituted with from one to three fluoro atoms and can also be optionally substituted with an amino or hydroxy substituent, provided that when Q is N, R¹ is absent and when Y is N, R is absent;
R⁴, R⁵, R⁶, R⁷, R⁸, and R⁹ are independently selected from hydrogen, fluoro, hydroxy, (C₁-C₄) alkyl, and (C₁-C₄) alkoxy, wherein the alkyl moieties of the (C₁-C₄) alkyl or (C₁-C₄) alkoxy are straight or branched; provided that when Z is N, R⁸ cannot be fluoro or hydroxy;
R¹⁰ is independently selected from hydrogen, (C₁-C₄) alkyl, and (C₁-C₄) alkoxy, wherein the alkyl moieties of the (C₁-C₄) alkyl or (C₁-C₄) alkoxy are straight or branched;
R¹¹, R¹², R¹³, R¹⁴, and R¹⁵ are independently selected from hydrogen, halo, —(═O)CH₃, (C₁-C₄) alkyl, and (C₁-C₄) alkoxy, aryl, and aryloxy, wherein the alkyl moieties of the (C₁-C₄) alkyl, (C₁-C₄) alkoxy, and —C(═O)CH₃ groups and the aryl and aryloxy moieties can be optionally substituted with from one to three fluoro atoms and can also be optionally substituted with an amino or hydroxy substituent;
R¹⁶ and R¹⁷ are independently selected from hydrogen, halo, cyano, oxo, hydroxy, —C(═O)CH₃, (C₁-C₄) alkyl, and (C₁-C₄) alkoxy, wherein the alkyl moieties of the (C₁-C₄) alkyl, (C₁-C₄) alkoxy, and —C(═O)CH₃ groups can be optionally substituted with from one to three fluoro atoms and can also be optionally substituted with an amino or hydroxy substituent;

2. The compound or salt of claim 1, wherein D is N.

3. The compound or salt of claim 1, wherein at least one of Q and Z is N.

4. The compound or salt of claim 1, wherein R¹, R², R³, R⁶, R⁷, and R¹⁰ are each H.

5. The compound or salt of claim 4, wherein R⁴, R⁵, R⁸, and R⁹ are each independently H or methyl.

6. The compound or salt of claim 1, wherein A is —(CH₂)ₘCH₂— or —(CH₂)ₘO— and m is an integer from 3 to 5.

7. The compound or salt of claim 1, wherein G is a group of formula (i), and V is C or CH.

8. The compound or salt of claim 1, wherein G is a group of formula (i), and R¹¹, R¹², and R¹³ are independently selected from the group consisting of halo, methyl, ethyl, isopropyl, and cyclopropyl.

9. The compound or salt of claim 1, wherein G is a group of formula (ii), and J and K are each C or CH.

10. The compound or salt of claim 1, wherein G is a group of formula (ii), and ring AA is an unsaturated 6-membered carbocyclic ring.

11. The compound or salt of claim 1, wherein G is a group of formula (ii), and R¹⁴ and R¹⁵ are both H.

12. The compound or salt of claim 1, wherein G is a group of formula (ii), and R¹⁶ and R¹⁷ are independently selected from the group consisting of H, F, methyl, CN, and methoxy.

13. The compound or salt of claim 1, wherein G is a flouro-naphthalenyl group.

14. The compound or salt of claim 1, selected from the group consisting of:
2-[4-(4-Naphthalen-1-yl-piperazin-1-yl)-butoxy]-5,6,7,9-tetrahydro-pyrido[2,3-b]azepin-8-one;
2-{4-[4-(2,3-dichloro-phenyl)-piperazin-1-yl]-butoxy}-5,6,7,9-tetrahydro-pyrido[2,3-b]azepin-8-one;
2-[4-(4-Chroman-8-yl-piperazin-1-yl)-butoxy]-5,6,7,9-tetrahydro-pyrido[2,3-b]azepin-8-one;
2-{4-[4-(5,6,7,8-Tetrahydro-naphthalen-1-yl)-piperazin-1-yl]-butoxy}-5,6,7,9-tetrahydro-pyrido[2,3-b]azepin-8-one;
2-[4-(4-Indan-4-yl-piperazin-1-yl)-butoxy]-5,6,7,9-tetrahydro-pyrido[2,3-b]azepin-8-one;
2-{4-[4-(2,3-Dihydro-benzofuran-7-yl)-piperazin-1-yl]-butoxy}-5,6,7,9-tetrahydro-pyrido[2,3-b]azepin-8-one;
2-{4-[4-(7-Fluoro-naphthalen-1-yl)-piperazin-1-yl]-butoxy}-5,6,7,9-tetrahydro-pyrido[2,3-b]azepin-8-one;
2-{4-[4-(3,4-Dihydro-2H-benzo[b][1,4]dioxepin-6-yl)-piperazin-1-yl]-butoxy}-5,6,7,9-tetrahydro-pyrido[2,3-b]azepin-8-one;
8-{4-[4-(8-Oxo-6,7,8,9-tetrahydro-5H-pyrido[2,3-b]azepin-2-yloxy)-butyl]-piperazin-1-yl}-naphthalene-2-carbonitrile;
2-{4-[4-(1-Methyl-2-oxo-1,2,3,4-tetrahydro-quinolin-8-yl)-piperazin-1-yl]-butoxy}-5,6,7,9-tetrahydro-pyrido[2,3-b]azepin-8-one;
2-[4-(4-Indan-4-yl-piperazin-1-yl)-butoxy]-5,6,7,9-tetrahydro-1,7,9-triaza-benzocyclohepten-8-one;
2-{4-[4-(2,3-Dichloro-phenyl)-piperazin-1-yl]-butoxy}-5,6,7,9-tetrahydro-1,7,9-triaza-benzocyclohepten-8-one;
2-[4-(4-Naphthalen-1-yl-piperazin-1-yl)-butoxy]-5,6,7,9-tetrahydro-1,7,9-triaza-benzocyclohepten-8-one;
2-{4-[4-(5,6,7,8-Tetrahydro-naphthalen-1-yl)-piperazin-1-yl]-butoxy}-5,6,7,9-tetrahydro-1,7,9-triaza-benzocyclohepten-8-one;
2-[4-(4-Chroman-8-yl-piperazin-1-yl)-butoxy]-5,6,7,9-tetrahydro-1,7,9-triaza-benzocyclohepten-8-one;
2-{4-[4-(7-Fluoro-naphthalen-1-yl)-piperazin-1-yl]-butoxy}-5,6,7,9-tetrahydro-1,7,9-triaza-benzocyclohepten-8-one;
2-{4-[4-(2,3-Dihydro-benzofuran-7-yl)-piperazin-1-yl]-butoxy}-5,6,7,9-tetrahydro-1,7,9-triaza-benzocyclohepten-8-one;
2-{4-[4-(3,4-Dihydro-2H-benzo[b][1,4]dioxepin-6-yl)-piperazin-1-yl]-butoxy}-5,6,7,9-tetrahydro-1,7,9-triaza-benzocyclohepten-8-one;
2-{4-[4-(7-Methoxy-naphthalen-1-yl)-piperazin-1-yl]-butoxy}-5,6,7,9-tetrahydro-1,7,9-triaza-benzocyclohepten-8-one;
8-{4-[4-(8-Oxo-6,7,8,9-tetrahydro-5H-1,7,9-triaza-benzocyclohepten-2-yloxy)-butyl]-piperazin-1-yl}-naphthalene-2-carbonitrile;
2-{4-[4-(1-Methyl-2-oxo-1,2,3,4-tetrahydro-quinolin-8-yl)-piperazin-1-yl]-butoxy}-5,6,7,9-tetrahydro-1,7,9-triaza-benzocyclohepten-8-one;
8-{4-[4-(2,3-Dichlorophenyl)piperazin-1-yl]butoxy}-3-methyl-1,3,4,5-tetrahydro-benzo[d][1,3]diazepin-2-one;
8-{3-[4-(2,3-Dichlorophenyl)piperazin-1-yl]propoxy}-1,3,4,5-tetrahydrobenzo[d][1,3]diazepin-2-one;
8-{4-[4-(2,3-Dichlorophenyl)piperazin-1-yl]butoxy}-1,3,4,5-tetrahydrobenzo[d][1,3]diazepin-2-one;
8-{4-[4-(2-Chloro-4-fluoro-3-methyl-phenyl)-piperazin-1-yl]-butoxy}-1,3,4,5-tetrahydro-benzo[d][1,3]diazepin-2-one;
8-{4-[4-(2-Chloro-4-fluoro-5-methyl-phenyl)-piperazin-1-yl]-butoxy}-1,3,4,5-tetrahydro-benzo[d][1,3]diazepin-2-one;
8-[4-(4-Naphthalen-1-yl-piperazin-1-yl)-butoxy]-1,3,4,5-tetrahydro-benzo[d][1,3]diazepin-2-one;
8-{4-[4-(6-Ethyl-pyridin-2-yl)-piperazin-1-yl]-butoxy}-1,3,4,5-tetrahydro-benzo[d][1,3]diazepin-2-one;
8-{4-[4-(6-Isopropyl-pyridin-2-yl)-piperazin-1-yl]-butoxy}-1,3,4,5-tetrahydro-benzo[d][1,3]diazepin-2-one;
8-{4-[4-(2-Chloro-4-fluoro-phenyl)-piperazin-1-yl]-butoxy}-1,3,4,5-tetrahydro-benzo[d][1,3]diazepin-2-one;
8-{4-[4-(2,3-Dichloro-4-fluoro-phenyl)-piperazin-1-yl]-butoxy}-1,3,4,5-tetrahydro-benzo[d][1,3]diazepin-2-one;
8-{4-[4-(6-Cyclopropyl-pyridin-2-yl)-piperazin-1-yl]-butoxy}-1,3,4,5-tetrahydro-benzo[d][1,3]diazepin-2-one;
8-{4-[4-(7-Fluoro-naphthalen-1-yl)-piperazin-1-yl]-butoxy}-1,3,4,5-tetrahydro-benzo[d][1,3]diazepin-2-one;
8-{4-[4-(2,1,3-benzothiadiazol-4-yl)piperazin-1-yl]butoxy}-1,3,4,5-tetrahydro-2H-1,3-benzodiazepin-2-one;
8-{4-[4-(5-Fluoro-naphthalen-1-yl)-piperazin-1-yl]-butoxy}-1,3,4,5-tetrahydro-benzo[d][1,3]diazepin-2-one;
8-{3-[4-(2-Methoxy-quinolin-8-yl)-piperazin-1-yl]-propoxy}-1,3,4,5-tetrahydro-benzo[d][1,3]diazepin-2-one;
8-{4-[4-(8-Fluoro-naphthalen-1-yl)-piperazin-1-yl]-butoxy}-1,3,4,5-tetrahydro-benzo[d][1,3]diazepin-2-one;
8-[3-(4-Naphthalen-1-yl-piperazin-1-yl)-propoxy]-1,3,4,5-tetrahydro-benzo[d][1,3]diazepin-2-one;
8-{3-[4-(7-Fluoro-naphthalen-1-yl)-piperazin-1-yl]-propoxy}-1,3,4,5-tetrahydro-benzo[d][1,3]diazepin-2-one;
8-[4-(4-Isochroman-8-yl-piperazin-1-yl)-butoxy]-1,3,4,5-tetrahydro-benzo[d][1,3]diazepin-2-one;
8-{3-[4-(2,3-Dichlorophenyl)piperazin-1-yl]-propoxy}-5,5-dimethyl-1,3,4,5-tetrahydrobenzo[b]azepin-2-one;
8-{4-[4-(2,3-Dichlorophenyl)piperazin-1-yl]-butoxy}-5,5-dimethyl-1,3,4,5-tetrahydrobenzo[b]azepin-2-one;
8-{5-[4-(2,3-Dichlorophenyl)piperazin-1-yl]-pentyloxy}-5,5-dimethyl-1,3,4,5-tetrahydrobenzo[b]azepin-2-one;
8-{4-[4-(2,3-Dichlorophenyl)piperazin-1-yl]butoxy}-3,3-dimethyl-1,3,4,5-tetrahydrobenzo[b]azepin-2-one;
4,4-Dimethyl-8-[4-(4-naphthalen-1-yl-piperazin-1-yl)-butoxy]-1,3,4,5-tetrahydro-benzo[d][1,3]diazepin-2-one;
4,4-Dimethyl-8-[3-(4-naphthalen-1-yl-piperazin-1-yl)-propoxy]-1,3,4,5-tetrahydro-benzo[d][1,3]diazepin-2-one;
8-{4-[4-(2,3-Dichloro-phenyl)-piperazin-1-yl]-butoxy}-1,3,4,5-tetrahydro-benzo[b]azepin-2-one;
8-{5-[4-(2,3-Dichloro-phenyl)-piperazin-1-yl]-pent-1-enyl}-1,3,4,5-tetrahydro-benzo[d][1,3]diazepin-2-one;
8-{5-[4-(2,3-Dichloro-phenyl)-piperazin-1-yl]-pentyl}-1,3,4,5-tetrahydro-benzo[d][1,3]diazepin-2-one;
8-{5-[4-(2,3-Dichlorophenyl)piperazin-1-yl]pentyl}-5,5-dimethyl-1,3,4,5-tetrahydro-benzo [d][1,3]diazepin-2-one;
8-{5-[4-(2,3-Dichlorophenyl)-piperazin-1-yl]pent-1-enyl}-5,5-dimethyl-1,3,4,5-tetrahydrobenzo[b]azepin-2-one;
8-{5-[4-(2,3-Dichlorophenyl)piperazin-1-yl]pentyl}-5,5-dimethyl-1,3,4,5-tetrahydrobenzo[b]azepin-2-one; or
a pharmaceutically acceptable salt of any of said compounds.

15. A pharmaceutical composition, comprising:
(a) a compound according to claim 1, or a pharmaceutically acceptable salt thereof, and
(b) a pharmaceutically acceptable carrier.

16. A pharmaceutical composition, comprising:
(a) a compound according to claim 1, or a pharmaceutically acceptable salt thereof; and
(b), an antidepressant or an anti-anxiety agent, or a pharmaceutically acceptable salt thereof.

17. A method of treating a central nervous system disorder or condition in a mammal, comprising administering to the mammal an effective amount of a compound or salt according to claim 1.

18. The method of claim 17, wherein the mammal is a human.

19. The method of claim 18, wherein the central nervous system disorder or condition is selected from the group consisting of: major depression, single episode depression, recurrent depression, child abuse induced depression, postpartum depression, dysthymia, cyclothymia, bipolar disorder, delusional disorder, substance-induced psychotic disorder, brief psychotic disorder, shared psychotic disorder, psychotic disorder due to a general medical condition, autism, pervasive development disorder, attention deficit hyperactivity disorder, generalized anxiety disorder, panic disorder, obsessive-compulsive disorder, post-traumatic stress disorder, phobias, schizophrenia, schizophrenia with concomitant depression, schizophrenia with concomitant anxiety, and schizoaffective disorder.

20. The method of claim 19, wherein the central nervous system disorder or condition is selected from the group consisting of: major depression, single episode depression, recurrent depression, child abuse induced depression, postpartum depression, dysthymia, cyclothymia, and bipolar disorder.

21. The method of claim 19, wherein the central nervous system disorder or condition is selected from the group consisting of: schizophrenia, schizoaffective disorder, delusional disorder, substance-induced psychotic disorder, brief psychotic disorder, shared psychotic disorder, and psychotic disorder due to a general medical condition.

22. The method of claim 19, wherein the central nervous system disorder or condition is selected from the group consisting of autism, pervasive development disorder, and attention deficit hyperactivity disorder.

23. The method of claim 19, wherein the central nervous system disorder or condition is selected from the group consisting of: generalized anxiety disorder, panic disorder, obsessive-compulsive disorder, post-traumatic stress disorder, and phobias.

24. The method of claim 19, wherein the central nervous system disorder or condition is schizophrenia.

25. The method of claim 19, wherein the central nervous system disorder or condition is bipolar disorder.

26. The method of claim 19, wherein the central nervous system disorder or condition is schizophrenia with concomitant depression.

27. The method of claim 19, wherein the central nervous system disorder or condition being treated is schizophrenia with concomitant anxiety.

28. A method for treating a disorder or condition in a mammal, comprising administering to the mammal in need of such treatment:
(a) an effective amount of a compound according to claim 1, or a pharmaceutically acceptable salt thereof; and
(b) an effective amount of an antidepressant or an anti-anxiety agent,
wherein the disorder or condition is selected from the group consisting of: major depressive disorders, dysthymic disorders, depressive neurosis and neurotic depression, melancholic depression, atypical depression, bipolar disorder, cyclothymic disorder, conduct disorder, disruptive behavior disorder, attention deficit hyperactivity disorder, anxiety disorders, borderline personality disorder, schizophrenia, neurodegenerative disorders, movement disorders, dyskinesias, chemical dependencies and additions, behavioral addictions, and ocular disorders.
